(19) United States
(12) Patent Application Publication
(10) Pub. No.: US 2025/0387519 A1
(43) Pub. Date: Dec. 25, 2025

(54) MECP2 BASED THERAPY

(71) Applicants: THE UNIVERSITY COURT OF THE UNIVERSITY OF EDINBURGH, Edinburgh (GB); THE UNIVERSITY COURT OF THE UNIVERSITY OF GLASGOW, Glasgow (GB)

(72) Inventors: Adrian BIRD, Edinburgh (GB); Rebekah TILLOTSON, Edinburgh (GB); Stuart Robert COBB, Edinburgh (GB); Ralph David HECTOR, Edinburgh (GB)

(21) Appl. No.: 19/051,709

(22) Filed: Feb. 12, 2025

Related U.S. Application Data
(63) Continuation of application No. 16/497,271, filed on Sep. 24, 2019, now abandoned.

(30) Foreign Application Priority Data
Mar. 24, 2017 (GB) 1704704.4
Mar. 24, 2017 (GB) 1704722.6

(51) Int. Cl. A61K 48/00; A61K 9/00; A61K 38/00; A61P 25/14; C07K 14/00; C07K 14/47; C12N 7/00; C12N 15/86

(52) U.S. Cl. CPC A61K 48/0066; A61K 9/0019; A61K 9/0085; A61P 25/14; C07K 14/001; C07K 14/4703; C12N 7/00; C12N 15/86; A61K 38/00; C12N 2750/14143

(57) ABSTRACT

MeCP2 based therapy. The present invention relates to synthetic polypeptides that are useful in the treatment of disorders associated with reduced MeCP2 activity, including Rett syndrome. The present invention provides synthetic polypeptides comprising: i) an MBD amino acid sequence showing at least 70% similarity with the amino acid sequence as depicted in SEQ ID NO: 1; and ii) an NID amino acid sequence showing at least 70% similarity with the amino acid sequence as depicted in SEQ ID NO: 2, wherein the polypeptide has a deletion of at least 50 amino acids, when compared to the full length MeCP2 e1 and e2 sequences. The invention further provides nucleic acid constructs, expression vectors, virions, pharmaceutical compositions, and cells providing polynucleotides of the invention. The invention further provides methods of treating or preventing disease in an animal comprising administering to said animal a synthetic polypeptide according to the invention.

This application is a continuation of U.S. patent application Ser. No. 16/497,271, which is a national stage application under 35 U.S.C. § 371 of PCT Application No. PCT/GB2018/050772, filed Mar. 23, 2018, which claims priority of Great Britain Patent Application No. 1704704.4, filed Mar. 24, 2017, and Great Britain Patent Application No. 1704722.6, filed Mar. 24, 2017.

### SEQUENCE LISTING STATEMENT

The Sequence Listing is being submitted electronically in XML format and is hereby incorporated by reference in its entirety. Said XML copy, created on Feb. 11, 2025, is named 136526001302.xml and is 115,006 bytes in size. No new matter is being introduced.

### FIELD OF THE INVENTION

The present invention relates to synthetic polypeptides that are useful in the treatment of disorders associated with reduced MeCP2 activity, including Rett syndrome. The invention also relates to nucleic acid constructs, expression vectors, virions and cells for expressing the synthetic polypeptides. Further, the invention concerns methods of treating disorders, such as Rett syndrome, using the synthetic polypeptides of the invention, the use of the synthetic polypeptides, nucleic acid constructs, expression vectors, virions and cells in the manufacture of medicaments for the treatment of disorders associated with reduced MeCP2 activity, including Rett syndrome, and pharmaceutical compositions comprising the synthetic polypeptides, nucleic acid constructs, expression vectors, and virions of the invention.

### BACKGROUND TO THE INVENTION

Methyl CpG-binding Protein 2 (MeCP2) is a nuclear protein that was named for its ability to preferentially bind methylated DNA. Interest in MeCP2 increased when mutations in the MECP2 gene were identified in the majority of Rett syndrome patients.

Rett syndrome occurs in about 1 in 15,000 girls. Although it is in theory a rare disease because it affects fewer than 1 in 2000 individuals, it is actually one of the most common genetic causes of intellectual disability in women. It was originally considered to be a neurodevelopmental disorder, due to the decreased, arrested and retarded development of those with the disorder from the age of about 6 months. However, the fact that some of the main symptoms have been found to be reversible in a mouse model of the disease means that it is now generally considered to be a neurological disorder.

The MECP2 gene is located on the X chromosome. It spans 76 kb and is composed of four exons. The MeCP2 protein has two isoforms, MeCP2 e1 and MeCP2 e2, which differ at the N-terminus of the protein. The isoform e1 is made up of 498 amino acids and isoform e2 is 486 amino acids long. The MECP2 (human) and Mecp2 (mouse) genes consist of four exons and undergo alternative splicing to produce the two mRNA species: e1 consists of exons 1,3 and 4; and e2 consists of 1, 2, 3 and 4. Translation starts from exon 1 or 2 in isoforms e1 and e2, respectively. Since the vast majority of the coding sequence is in exons 3 and 4, these two isoforms are very similar and only differ at the extreme N-termini. The mRNA of the MECP2 e1 variant has greater expression in the brain than that of the MECP2 e2 and the e1 protein is more abundant in the mouse and human brain. MeCP2 is an abundant mammalian protein that selectively binds 5-methyl cytosine residues in symmetrically methylated mCpG dinucleotides and asymmetrically methylated mCpA dinucleotides. CpG dinucleotides are preferentially located in the promoter regions of genes, but these are mostly unmethylated. In comparison, it is the CpG dinucleotides in the bulk genome that are highly methylated and it is to these that MeCP2 binds. The presence of mCpA methylated in neurons further increases the number of binding sites. In this way, MeCP2 regulates gene transcription by binding in the main body of gene sequences¹.

MeCP2 is highly conserved across vertebrates, and at least six biochemically distinct domains have been identified in the protein², including High Mobility Group Protein-like Domains, the Methyl Binding Domain (MBD), the Transcriptional Repression Domain comprising the NCoR/SMRT Interaction Domain (NID), and the C-terminal domains α and β. Functionally, MeCP2 has been implicated in several cellular processes based on its reported interaction with >40 binding partners³, including transcriptional co-repressors⁴ (e.g. NCoR/SMRT⁵), transcriptional activators⁶, RNA⁷, chromatin remodellers^{8,9}, microRNA-processing proteins¹⁰, and splicing factors¹¹. Accordingly, MeCP2 has been cast as a multi-functional hub that integrates diverse functions that are essential in mature neurons¹².

There are currently no treatments available that are specific for Rett syndrome. Instead, treatment generally involves treating the symptoms of the disease using traditional drugs, whilst preventative strategies involve aggressive nutritional management, prevention of gastrointestinal and orthopedic complications, and rehabilitation therapies. Thus there remains a pressing need for a means of specifically treating and preventing the development of Rett syndrome.

### SUMMARY OF THE INVENTION

Accordingly, the present invention provides synthetic polypeptides comprising an MBD amino acid sequence showing at least 70% similarity with the amino acid sequence as depicted in SEQ ID NO: 1 and an NID amino acid sequence showing at least 70% similarity with the amino acid sequence as depicted in SEQ ID NO: 2.

The synthetic polypeptides of the invention may have a deletion or substitution of at least 50 amino acids compared to the full length MeCP2 e1 and e2 sequences (SEQ ID NOs 3 and 4). Additionally, or alternatively, the synthetic polypeptides of the invention have less than 90% identity over the entire length of the amino acid sequence of MeCP2 as depicted in SEQ ID NO: 3 and/or SEQ ID NO: 4.

Generally, the synthetic polypeptides of the invention will comprise MBD and NID domains in accordance with MeCP2, but will be lacking other parts of the natural sequence of MeCP2. Thus any deletion or substitution in the synthetic polypeptide may be of a part of the natural sequence of MeCP2, but not of a part of the MBD or NID of MeCP2. Thus the synthetic polypeptides of the invention may generally have the structure

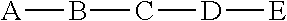

wherein portion B of the synthetic polypeptide is the MBD amino acid sequence showing at least 70% similarity with the amino acid sequence as depicted in SEQ ID NO: 1, and portion D of the synthetic polypeptide is the NID amino acid sequence showing at least 70% similarity with the amino acid sequence as depicted in SEQ ID NO: 2, and further wherein at least one of the three following are true: portion A of the synthetic polypeptide is less than 30 amino acids long and/or has less than 90% identity to the amino acid sequences as depicted in SEQ ID NOs: 5 and 6, calculated over the entire length of the amino acid sequences as depicted in SEQ ID NO: 5 and 6; portion C of the synthetic polypeptide is less than 20 amino acids long and/or has less than 90% identity to the amino acid sequence as depicted in SEQ ID NO: 7, calculated over the entire length of the amino acid sequence as depicted in SEQ ID NO: 7; and portion E of the synthetic polypeptide is absent, a polypeptide tag, and/or has less than 90% identity to the amino acid sequence as depicted in SEQ ID NO: 8, calculated over the entire length of the amino acid sequence as depicted in SEQ ID NO: 8. The skilled person will appreciate that this general structure is disclosed from left to right in the accepted N-terminal to C-terminal direction of the synthetic polypeptide.

The invention also provides nucleic acid constructs encoding a synthetic polypeptide of the invention, and expression vectors comprising a nucleotide sequence encoding a synthetic polypeptide of the invention. The expression vector may be a viral vector, and thus the invention also provides a virion comprising an expression vector according to the invention. The invention also provides cells that comprise a synthetic genetic construct adapted to express a polypeptide of the invention, cells comprising a vector of the invention, and cells for producing a virion of the invention.

The invention also provides pharmaceutical compositions comprising the synthetic polypeptides, nucleic acids constructs, expression vectors and/or virions of the invention.

The synthetic polypeptides of the invention have utility in medicine, and particularly in the treatment of neurological disorders associated with inactivation, such as an inactivating mutation, of MECP2. Such disorders include Rett syndrome. Therefore the invention provides a method of treating or preventing disease in an animal comprising administering to said animal a synthetic polypeptide of the invention. Said administering may comprise administering a synthetic polypeptide of the invention, an expression vector of the invention, a virion of the invention and/or a pharmaceutical composition of the invention.

Furthermore, the invention provides synthetic polypeptides of the invention, expression vectors of the invention, and virions of the invention for the treatment or prevention of a neurological disorder associated with inactivating mutation of MECP2, for example Rett syndrome. The invention also provides the use of synthetic polypeptides of the invention, expression vectors of the invention, and virions of the invention in the manufacture of a medicament for the treatment or prevention of a neurological disorder associated with inactivating mutation of MECP2, for example Rett syndrome.

### DETAILED DESCRIPTION

The synthetic polypeptides of the invention provide therapeutic proteins that can be used in the treatment of disorders that are caused by inactivation or reduced activity of MeCP2. Such disorders include, in particular, Rett syndrome. The nucleic acid constructs, expression vectors, virions and cells of the invention can be used to produce and, optionally, deliver the synthetic polypeptides. The invention also provides methods of treatment using the products of the invention, and the use of those products in those treatments.

The invention is based on the inventors' surprising and unexpected finding that it is a deficiency in the biological activity associated with the MBD and NID of MeCP2 that is key to the development of Rett syndrome, such that other parts of the protein are not necessary, despite the fact that MeCP2 is a highly conserved protein. They generated and tested the hypothesis that the MeCP2 functions that are vital in Rett syndrome are those due to MeCP2 forming a bridge between chromatin and the NCoR/SMRT complex, so that all other domains of MeCP2 are dispensable. Furthermore, they hypothesised that the Rett syndrome mutations occurring within the MBD or NID domains interfere directly with this function, and that Rett syndrome mutations occurring outside the MBD and NID either destabilise the protein generally or specifically impair the bridge between the chromatin and the NCoR/SMRT complex. As a result of their studies and understanding, they have concluded that the MBD and the NID are therefore sufficient for the MeCP2 function required to treat or prevent Rett syndrome and similar disorders. This means that they are able to provide a “mini-MeCP2” protein derivative by jettisoning a significant portion, for example in some embodiments up to 50-65%, of the native MeCP2 protein.

As discussed elsewhere in the specification, the inventors' conclusion means that therapeutic synthetic polypeptides can be prepared that are considerably smaller than the full length MeCP2. Of course, this means that they can be easier to produce and effectively deliver to patients. For example, some delivery vehicles, such as adeno-associated viral (AAV) vectors, are restricted as to the amount of payload they can carry, so the ability to lighten that load by encoding a smaller polypeptide is advantageous. Also, the removal of unnecessary parts of the MeCP2 polypeptide means that alternative polypeptide sequence, such as peptide tags, regulatory tags and/or signaling peptides can be inserted in the polypeptide in some embodiments without making the polypeptide overly large. Similarly, the smaller protein means that a smaller nucleic acid sequence can encode the protein, such that when there are size constraints on the amount of nucleic acid sequence that can be included in a particular construct in some embodiments of the invention, the constructs of that type that encode the polypeptides of the invention may include additional sequences, such as regulatory elements, that would be difficult to include if the full-length MeCP2 protein was being encoded instead of the smaller polypeptide, due to the size constraints. Furthermore, the removal of other biologically active, but unnecessary, parts of the MeCP2 protein means that there may be less chance of unwanted side-effects due to interactions of those parts of the protein during the therapeutic or preventative treatment.

Thus the invention provides improved methods of treating or preventing disorders associated with reduced MeCP2 activity, such as Rett syndrome, as well as therapeutic products for use in those methods.

Furthermore, the inventors have surprisingly and unexpectedly found that although deletion of the part of MeCP2 that links the MBD and NID domains appears to reduce the stability of the synthetic polypeptide having the MBD and NID domains, this reduced stability can have a beneficial effect as it reduces the toxicity that can be associated with over-dosing of subjects with a MeCP2 polypeptide. Thus the invention also provides in some embodiments of the invention improved methods that are safer and less likely to be associated with toxic side effects, as well as the therapeutic products for use in those methods, which provide synthetic polypeptides lacking at least part of the amino acid sequence that links the MBD and NID in MeCP2.

In order to assist the understanding of the present invention, certain terms used herein will now be further defined, and more generally further details of the invention will be given, in the following paragraphs.

### Synthetic Polypeptides

The invention provides synthetic polypeptides comprising an MBD amino acid sequence and an NID amino acid sequence.

As used herein, the term “polypeptide” can be used interchangeably with “peptide” or “protein”, and means at least two covalently attached alpha amino acid residues linked by a peptidyl bond. The term polypeptide encompasses purified natural products, or chemical products, which may be produced partially or wholly using recombinant or synthetic techniques. The term polypeptide may refer to a complex of more than one polypeptide, such as a dimer or other multimer, a fusion protein, a protein variant, or derivative thereof. The term also includes modified proteins, for example, a protein modified by glycosylation, acetylation, phosphorylation, pegylation, ubiquitination, and so forth. A polypeptide may comprise amino acids not encoded by a nucleic acid codon.

As used herein, the term “synthetic polypeptide” refers to polypeptide sequences formed by processes through human agency. The synthetic polypeptides of the invention are based on MeCP2 in that they have biologically active MBD and NID sequences, such as those that occur in wild type MeCP2 proteins, but are distinguished from the naturally occurring MeCP2 proteins. The polypeptides of the invention are synthetic because they include mutations, such as amino acid deletions, substitutions and/or insertions, in the wild type MeCP2 sequences such that the resultant synthetic polypeptides are not known from the art as natural polypeptides.

“Naturally occurring,” “native,” or “wild-type” is used to describe an object that can be found in nature as distinct from being artificially produced. For example, a protein or nucleotide sequence present in an organism (including a virus), which can be isolated from a source in nature and that has not been intentionally modified by a person in the laboratory, is naturally occurring.

The Methyl-CpG Binding Domain (MBD) of MeCP2 has the ability to bind methylated DNA. The human MeCP2 MBD sequence is provided herein as SEQ ID NO: 1. It consists of amino acids 72 to 173, inclusive, of the human MeCP2 protein (numbering refers to the e2 isoform, i.e. as in SEQ ID NO: 4). The mouse MeCP2 MBD sequence is identical to the human sequence. The polypeptides of the invention comprise an MBD having at least 70% similarity to this MeCP2 MBD sequence (SEQ ID NO: 1). Preferably the polypeptides of the invention comprise an MBD having at least 70%, 75%, 80%, 85%, 88%, 90%, 92%, 94%, 95%, 96%, 97%, 98% or 99% similarity to the human MeCP2 MBD sequence (SEQ ID NO:1). Further preferably the polypeptides of the invention comprise an MBD having at least 90% similarity. Most preferably the polypeptides of the invention comprise the human MeCP2 MBD sequence (SEQ ID NO:1). The MBD sequences of the synthetic polypeptides of the invention have the ability to bind methylated DNA.

The MBD sequence of particular interest for the synthetic polypeptides of the invention is that of the amino acids at positions 78 to 162 of the MeCP2 e2 isoform (SEQ ID NO: 4). Thus in preferred embodiments of the invention the polypeptides of the invention comprise an MBD having at least 85%, 88%, 90%, 92%, 94%, 95%, 96%, 97%, 98% or 99% similarity to the sequence of amino acids from positions 78 to 162 of the MeCP2 e2 isoform (SEQ ID NO: 4). Most preferably MBD amino acid sequences of the polypeptides of the invention comprise the sequence of amino acids from positions 78 to 162 of the MeCP2 e2 isoform (SEQ ID NO: 4).

The MBD sequence of MeCP2 includes several phosphorylation sites (Ser80, Ser86, Thr148/9 and Ser164; numbering with respect to the e2 isoform). Phosphorylation at Ser80 and Ser164, at least, has been associated with affecting the activity of MeCP2. Therefore it is preferred that one, more, or all of these amino acids are retained in the MBD sequences of the synthetic polypeptides of the invention.

The MBD sequence of the invention may correspond to that of a naturally occurring MeCP2 MBD sequence, for example the sequence of MBD in the zebrafish homolog of MeCP2.

The NCoR/SMRT Interaction Domain (NID) of MeCP2 is the domain through which MeCP2 interacts with the NCoR/SMRT co-repressor complexes. The human MeCP2 NID sequence is provided herein as SEQ ID NO: 2. It consists of amino acids 272 to 312, inclusive, of the human MeCP2 protein (numbering refers to the e2 isoform, i.e. as in SEQ ID NO: 4). The mouse MeCP2 MBD sequence is identical to the human sequence, except for amino acid position 297 in SEQ ID NO: 4 (i.e. the amino acid at position 26 in SEQ ID NO: 2), which is histidine in mouse but glutamine in human. The polypeptides of the invention comprise an NID amino acid sequence having at least 70% similarity to this MeCP2 NID sequence (SEQ ID NO: 2). Preferably the polypeptides of the invention comprise an NID having at least 75%, 80%, 85%, 88%, 90%, 92%, 94%, 95%, 95%, 97%, 98% or 99% similarity to the human MeCP2 NID sequence (SEQ ID NO: 2). Further preferably the polypeptides of the invention comprise an NID having at least 90% similarity. Most preferably the polypeptides of the invention comprise the human MeCP2 NID sequence (SEQ ID NO: 2). The NID sequences of the synthetic polypeptides of the invention have the ability to interact, or bind, with the NCoR/SMRT co-repressor complex.

The NID sequence of particular interest for the synthetic polypeptides of the invention is that of the amino acids at positions 298 to 309 of the MeCP2 e2 isoform (SEQ ID NO: 4). Thus in preferred embodiments of the invention the polypeptides of the invention comprise an MBD having at least 80%, 85%, 88%, 90%, 92%, 94%, 95%, 96%, 97%, 98% or 99% similarity to the sequence of amino acids from positions 298 to 309 of the MeCP2 e2 isoform (SEQ ID NO: 4). Most preferably NID amino acid sequences of the polypeptides of the invention comprise the sequence of amino acids from positions 298 to 309 of the MeCP2 e2 isoform (SEQ ID NO: 4).

The NID sequence of MeCP2 includes phosphorylation sites (Thr308 and Ser274; numbering with respect to the e2 isoform), the former of which has been associated with affecting the activity of the NID. Therefore it is preferred that Thr308 at least is retained in the NID sequences of the synthetic polypeptides of the invention.

The NID sequence of the invention may correspond to that of a naturally occurring MeCP2 NID sequence, for example the sequence of NID in the zebrafish homolog of MeCP2. The MBD and NID sequences may have the same amount of percentage similarity to their respective wild type human MeCP2 sequences, or they may have different amounts of percentage similarity to their respective wild type human MeCP2 sequences. The percentage similarities for the MBD and NID may therefore consist of any combination of the above disclosed percentage similarities. Thus the present invention provides synthetic polypeptides comprising an MBD amino acid sequence showing at least 70% similarity with the amino acid sequence as depicted in SEQ ID NO: 1 and an NID amino acid sequence showing at least 70% similarity with the amino acid sequence as depicted in SEQ ID NO: 2, but preferably the MBD and NID amino acid sequences may have at least 75%, 80%, 85%, 88%, 90%, 92%, 94%, 95%, 95%, 97%, 98% or 99% similarity to the human MeCP2 domain sequences. Further preferably at least 90% similarity. Similarly, the MBD sequence may have at least 70%, 75%, 80%, 85%, 88%, 90%, 92%, 94%, 95%, 95%, 97%, 98% or 99% similarity to the human MeCP2 MBD sequence whilst the NID sequence of the same synthetic polypeptide may have at least 70%, 75%, 80%, 85%, 88%, 90%, 92%, 94%, 95%, 95%, 97%, 98% or 99% similarity to the human MeCP2 NID sequence. Preferably one or both of the MBD and NID sequences will consist of or comprise their corresponding human or mouse domain sequence.

The term “similarity” refers to a degree of similarity between proteins or polypeptide sequences taking into account differences in amino acids at aligned positions of the sequences, but in which the functional similarity of the different amino acid residues, in view of almost equal size, lipophilicity, acidity, etc., is also taken into account. A percentage similarity can be calculated by optimal alignment of the sequences using a similarity-scoring matrix such as the Blosum62 matrix described in Henikoff S. and Henikoff J. G., P.N.A.S. USA 1992, 89: 10915-10919. Calculation of the percentage similarity and optimal alignment of two sequences using the Blosum62 similarity matrix and the algorithm of Needleman and Wunsch (J. Mol. Biol. 1970, 48: 443-453) can be performed using the GAP program of the Genetics Computer Group (GCG, Madison, WI, USA) using the default parameters of the program.

Exemplary parameters for amino acid comparisons for similarity in the present invention use the Blosum62 matrix (Henikoff and Henikoff, supra) in association with the following settings for the GAP program:
Gap penalty: 8
Gap length penalty: 2
No penalty for end gaps.

Functional polymorphic forms of MBD and NID from mice and humans, and homologues of these domains from MeCP2 of other species, may be included in the polypeptides of the present invention. Variants of these domains in the polypeptides that also form part of the present invention are natural or synthetic variants that may contain variations in the amino acid sequence due to deletions, substitutions, insertions, inversions or additions of one or more amino acids in said sequence or due to an alteration to a moiety chemically linked to a protein. For example, a protein variant may be an altered carbohydrate or PEG structure attached to a protein. The polypeptides of the invention may include at least one such protein modification.

“Variants” of a polypeptide domain or protein, as used herein, refers to a polypeptide domain or protein resulting when a polypeptide is modified by one or more amino acids (e.g. insertion, deletion or substitution), or which comprises a protein modification, or which contains modified or non-natural amino acids. Substitutional variants of polypeptides are those in which at least one residue in the amino acid sequence has been removed and a different residue inserted in its place. The domains in the polypeptides of the present invention can contain conservative changes, wherein a substituted amino acid has similar structural or chemical properties, or more rarely non-conservative substitutions, for example, replacement of a glycine with a tryptophan, as long as the domains retain function. Variants may also include sequences with amino acid deletions or insertions, or both. Guidance in determining which amino acid residues may be substituted, inserted, or deleted without abolishing biological or immunological activity may be found using computer programs well known in the art.

The term “conservative substitution”, relates to the substitution of one or more amino acid substitutions for amino acid residues having similar biochemical properties. Typically, conservative substitutions have little or no impact on the activity of a resulting polypeptide sequence. For example, a conservative substitution in a binding domain may be an amino acid substitution that does not substantially affect the ability of the domain to bind to its binding partner(s) or otherwise perform its usual biological function. Screening of variants of the polypeptide domains of the present invention can be used to identify which amino acid residues can tolerate an amino acid substitution. In one example, the relevant biological activity of a polypeptide having a modified domain is not altered by more than 25%, preferably not more than 20%, especially not more than 10%, when one or more conservative amino acid substitutions are effected.

One or more conservative substitutions can be included in a MBD or NID of a polypeptide of the present invention. In one example, 10 or fewer conservative substitutions are included in the domains. A polypeptide of the invention may therefore include 1, 2, 3, 4, 5, 6, 7, 8, 9, 10 or more conservative substitutions of the MBD and/or NID domains. A polypeptide can be produced to contain one or more conservative substitutions by manipulating the nucleotide sequence that encodes that polypeptide using, for example, standard procedures such as site-directed mutagenesis or PCR. Alternatively, a polypeptide can be produced to contain one or more conservative substitutions by using peptide synthesis methods, for example as known in the art.

Examples of amino acids which may be substituted for an original amino acid in a protein and which are regarded as conservative substitutions include: Ser for Ala; Lys for Arg; Gln or His for Asn; Glu for Asp; Asn for Gln; Asp for Glu; Pro for Gly; Asn or Gln for His; Leu or Val for lie; Ile or Val for Leu; Arg or Gln for Lys; Leu or lie for Met; Met, Leu or Tyr for Phe; Thr for Ser; Ser for Thr; Tyr for Trp; Trp or Phe for Tyr; and Ile or Leu for Val. In one embodiment, the substitutions are among Ala, Val, Leu and lie; among Ser and Thr; among Asp and Glu; among Asn and Gln; among Lys and Arg; and/or among Phe and Tyr. However, a substitution may not be considered conservative where it results in the removal of a site of phosphorylation within the polypeptide sequence. Further information about conservative substitutions can be found in, among other locations, Ben-Bassat et al., (J. Bacteriol. 169:751-7, 1987), O'Regan et al., (Gene 77:237-51, 1989), Sahin-Toth et al., (Protein Sci. 3:240-7, 1994), Hochuli et al., (Bio/Technology 6:1321-5, 1988), WO 00/67796 (Curd et al.) and in standard textbooks of genetics and molecular biology.

Substitutions causing loss or decrease of function of MBD and NID are known in the art, not least due to the association of some with Rett syndrome and other MeCP2 associated disorders. Examples of such harmful changes or mutations include those shown in Table 1 and FIG. 2C in the MBD, and those shown in Table 2 and FIG. 2D in NID. Thus the skilled person will understand that these harmful changes should not be included in the MBD and NID domains of the polypeptides of the invention.

TABLE 1
Harmful and benign amino acid changes in the MBD. According
to convention, all amino acid numbers given in the following
refer to the human and mouse e2 iosforms.

| Harmful changes associated with: | | Benign changes present in: | |
|---|---|---|---|
| Classical | Atypical RTT or other | General | Other [vertebrate] |
| RTT¹³ | intellectual disability¹³ | population¹⁴* | species** |
| L100V | S86C ♀ | P72L/S | P72L |
| L100R | P93S ♀ | P75L | A73S |
| P101R | D97Y/E ♀ | K82R | V74A |
| P101S | P101R ♀ | R85H | A77S |
| P101H | G103D ♀ | R89C | P81A |
| P101L | W104R ♀ | R91W | I87V |
| R106W | G114A ♀ | S113F | D96E |
| R106Q | Y120D ♀ | R115H | T99S |
| R106L | D121G ♀ | Q128E | E102Q |
| L108H | V122M ♀ | A140G | Y120F |
| R111G | N126S ♀ | K144R | Q128N/E |
| L124F | G129V ♀ | D147E/N | I139M |
| P127L | R133H/G ♀ | T160S | E143Q |
| Q128P | E137G ♂s only | K171N | S149I |
| A131D | A140V ♀s + ♂s | P172L | L150T |
| R133C | Y141C ♀ | P173A | Q170K |
| R133P | D151G ♀ | | K171R |
| R133L | P152A ♀s + ♂s | | P172Q |
| S134C | F155C ♀ | | |
| S134F | D156G ♀ | | |
| S134P | T160S ♀s + ♂s | | |
| K135E | G161E/W | | |
| L138S | P172S ♂s only | | |
| P152R | | | |
| F155S | | | |
| D156E | | | |
| D156A | | | |
| F157L | | | |
| F157I | | | |
| T158M | | | |
| T158A | | | |
| G161V | | | |

*those found in hemizygous males are in bold, and those in heterozygous females are in italics;
**those found in non-mammalian vertebrates are provided in italics. The “benign changes” listed below are not known to be associated with an MeCP2-associated disorder, therefore they are probably benign.

Tables 1 and 2 also list changes that have no known association with an MECP2-related disorder, and so that are believed to be benign. Thus the skilled person will understand that such apparently benign changes may optionally be included in synthetic polypeptides of the invention.

TABLE 2
Harmful and benign amino acid changes in the NID. According to
convention, all amino acid numbers given in the following refer
to the human and mouse e2 isoforms.

| Harmful changes associated with: | | Benign changes present in: | |
|---|---|---|---|
| Classical | Atypical RTT or other | General | Present in other |
| RTT¹³ | intellectual disability¹³ | population¹⁴* | [vertebrate] species |
| P302T | K286R ♀ | P272L | G273S/A |
| P302S | V300I ♀ | A278T/V | S274A |
| P302H | I303M ♀ | A279S/P | V275A/L |
| P302L | K304E/R ♀ | A291T | V276A |
| P302R | R309W ♀s + ♂s | A287V/P | A278I |
| K305R | T311M ♀ | V288M | A279L |
| K305T | | R294Q/P//G | A280T |
| R306C | | S295T | A281E |
| R306H | | T311A | E282A |
| | | | A288I/L |
| | | | I293A |
| | | | R294K |
| | | | S295P |
| | | | V296L |
| | | | Q297H(mouse)/L |
| | | | T299R |
| | | | V300A |
| | | | V312I/L |

*those found in hemizygous males are in bold, and those in heterozygous females are in italics;
**those found in mouse are in bold, and those found in non-mammalian vertebrates are provided in italics.

The biological activity of the MBD and NID domains that is of particular interest for the invention is the ability to recruit members of the NCoR/SMRT co-repressor complex to methylated DNA. Therefore it is preferred that the synthetic polypeptides of the invention are capable of recruiting NCoR/SMRT co-repressor complex components to methylated DNA. The NCoR/SMRT co-repressor complex components include NCoR, HDAC3, SIN3A, GPS2, SMRT, TBL1X and TBLR1. Preferably the synthetic polypeptides are capable of recruiting TBL1X or TBLR1 to methylated DNA.

The inventors have identified the MBD and NID domains as being key to the activity that is required of therapeutic MeCP2 in order for it to compensate for the reduced activity of MeCP2 in Rett syndrome and related disorders. Therefore whilst it is required that the MBD and NID domains are biologically active in the synthetic polypeptides of the invention, amino acid sequences in other parts of the wild type MeCP2 protein may be altered, for example by deletion of amino acids. Thus the synthetic polypeptides of the invention may have a deletion of at least 50 amino acids when compared to the full length human MeCP2 e1 and e2 sequences (SEQ ID NOs 3 and 4). Said deletion of at least 50 amino acids may be a deletion of at least 60, 70, 80, 90, 100, 125, 150, 175, 200, 225, 250, 275, or 300 amino acids when compared to the full length human MeCP2 e1 and e2 sequences (SEQ ID NOs 3 and 4). Preferably said deletion is of at least 200 amino acids when compared to the full length human MeCP2 e1 and e2 sequences (SEQ ID NOs 3 and 4). Such deletion of at least 50 amino acids may be assessed by preparing an alignment (see above) of the amino acid sequence of interest with the human MeCP2 e1 and e2 sequences (SEQ ID NOs 3 and 4). This will identify any regions in which amino acid residues in the MeCP2 sequences have been deleted because there will be gaps in the sequence of interest aligned to the MeCP2 e1 and e2 sequences (SEQ ID NOs 3 and 4). Preferably at least some of the amino acids that have been deleted will be consecutive within the MeCP2 sequence, such that said deletion of at least 50 amino acids will include the deletion of at least 5, 10, 15, 20, 30, 40, 50 or more consecutive amino acids within the MeCP2 e1 and e2 sequences (SEQ ID NOs 3 and 4). The deletion of the at least 50 amino acids will be apparent from the alignment with both the e1 and e2 sequences, therefore any deletion that is present in the N-terminal region of MeCP2 and that is only associated with one of the e1 and e2 sequences, should not be considered a deletion to be counted as part of the at least 50 consecutive amino acids deleted in accordance with the invention. However, a deletion that is present in the N-terminal region of MeCP2 and that is present in both the e1 and e2 sequence alignments should be considered a deletion and counted as part of the at least 50 deleted amino acids in accordance with the invention.

The amino acids deleted from the wild-type MeCP2 e1 and e2 sequences may be replaced with some other useful amino acid sequence. For example, a deletion of at least 50 amino acids may have occurred when compared to the full length human MeCP2 e1 and e2 sequences (SEQ ID NOs 3 and 4), but those deleted amino acids may have been replaced, at least in part, with amino acid sequence providing a linker, a tag and/or a signaling peptide. This may be identified in an alignment of the synthetic polypeptide with the MeCP2 e1 and e2 sequences by a stretch of amino acid sequence in the synthetic polypeptide that does not match the MeCP2 sequence, and wherein that stretch of unmatched amino acid sequence corresponds to a useful, or purposive, heterologous sequence. Thus the invention provides, in at least some embodiments, synthetic polypeptides that have MeCP2 activity associated with the MBD and NID sequences, but that can also include useful heterologous sequences without requiring the synthetic polypeptides to be larger than the wild type MeCP2 protein; since large parts of the MeCP2 sequence can be left out of the synthetic polypeptides of the invention, the heterologous sequence(s) can be included whilst maintaining a relatively small overall size for the synthetic polypeptide.

As an alternative to the above-mentioned deletion of at least 50 amino acids, or in addition to it, the synthetic polypeptide of the invention having the MBD and NID amino acid sequences may have alterations to the polypeptide amino acid sequences such that it has less than 90% identity to the amino acid sequences of MeCP2, as depicted in SEQ ID NOs: 3 and 4, over the entire length of the amino acid sequences of MeCP2, as depicted in SEQ ID NOs: 3 and 4. Said less than 90% identity will be apparent from the comparison with both the e1 and e2 sequences, therefore any such identity solely due to alterations in the N-terminal region of MeCP2, which are only associated with one of the e1 and e2 sequences, will not be considered as the synthetic polypeptide having less than 90% identity in accordance with the invention. Preferably said identity will be less than 85%, 80%, 75%, 70%, 65%, 60% or 55%. It is particularly preferred that said identity will be less than 60%.

Synthetic polypeptides of the invention will generally have the structure:

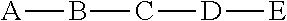

wherein portion B of the synthetic polypeptide is the MBD amino acid sequence, as described above, and portion D of the synthetic polypeptide is said NID amino acid sequence, as described above. As explained above, however, parts of the synthetic polypeptide other than the MBD and NID domains, i.e. portions A, C and D, may have alterations compared to the wild type MeCP2 sequence. Thus the synthetic polypeptide may include alterations in accordance with one or more of the following: portion A of the synthetic polypeptide is less than 40 amino acids long and/or has less than 95% identity to the amino acid sequences as depicted in SEQ ID NOs:5 and 6, calculated over the entire length of the amino acid sequences as depicted in SEQ ID NOs: 5 and 6; portion C of the synthetic polypeptide is less than 20 amino acids long and/or has less than 95% identity to the amino acid sequence as depicted in SEQ ID NO: 7, calculated over the entire length of the amino acid sequence as depicted in SEQ ID NO: 7; and portion E of the synthetic polypeptide is absent, a protein tag, and/or has less than 95% identity to the amino acid sequence as depicted in SEQ ID NO: 8, calculated over the entire length of the amino acid sequence as depicted in SEQ ID NO: 8.

Portion A of the synthetic polypeptide, corresponding to the N-terminal portion of the polypeptide and the area adjacent to the amino end of the MBD amino acid sequence when the MBD amino acid sequence is N-terminal to the NID amino acid sequence, may be less than 40 amino acids, preferably less than 35, 30, 25, or 20 amino acids. It is particularly preferred that portion A is less than 25 amino acids. Additionally or alternatively, portion A may have less than 95% identity to the amino acid sequences as depicted in SEQ ID NOs:5 and 6, calculated over the entire length of the amino acid sequences as depicted in SEQ ID NOs: 5 and 6; preferably the identity is less than 90%, less than 85%, less than 80%, less than 75%, less than 70%, less than 65%, less than 60%, or less than 50%. Preferably portion A is truncated compared to the natural sequences of MeCP2 e1 and e2, such that portion A is less than 72 amino acids long, preferably less than 70, 65, 50, 55, 45, 30, or 25 amino acids. Further preferably, portion A will be truncated to such an extent that SEQ ID NOs: 5 and 6 are essentially not present. For example, SEQ ID NOs: 5 and 6 may have been deleted from the amino acid sequence of the synthetic polypeptide, and optionally replaced with an N-terminal tag.

Thus the amino acid sequences specific to e1 and e2, which are 24 amino acids long in human e1 (29 amino acids long in mouse e1) and 9 amino acids long in e2 (human/mouse) and which are encoded by exons 1 and 2 and the first 10 base pairs of exon 3 of MECP2 and Mecp2, may be included in the synthetic polypeptides of the invention, and so, for example, may provide the most N-terminal amino acid sequence of the synthetic polypeptide. The mouse e1 specific N-terminal amino acid sequence is MAAAAATAAAAAAPSGGGGGGEEERLEEK (SEQ ID NO: 9). The mouse e2 specific N-terminal amino acid sequence is MVAGMLGLREEK (SEQ ID NO: 10). The human e1 specific N-terminal amino acid sequence is MAAAAAAAPSGGGGGGEEERLEEK (SEQ ID NO: 11). The human e2 specific N-terminal amino acid sequence is MVAGMLGLREEK (SEQ ID NO: 12). Therefore a synthetic polypeptide of the invention may comprise an amino acid sequence corresponding to any of SEQ ID NOs 9-12, and optionally said amino acid sequence may be the most N-terminal sequence in the synthetic polypeptide of the invention.

Preferably however, these extreme N-terminal sequences specific to the wild type e1 and e2 MeCP2 will not be included in the synthetic polypeptides of the invention. Therefore preferably a synthetic polypeptide of the invention will not comprise an amino acid sequence corresponding to any of SEQ ID NOs 9-12.

Preferably the amino acid sequence adjacent to the amino end of the MBD amino acid sequence has less than 75% identity to the amino acid sequences as depicted in SEQ ID NOs: 5 and 6, calculated over the entire length of the amino acid sequences as depicted in SEQ ID NOs: 5 and 6 preferably less than 50%, and further preferably less than 30% identity.

Preferably the amino acid sequence adjacent to the amino end of the MBD amino acid sequence is less than 50 amino acids long, preferably less than 30 amino acids long or less than 20 amino acids long, and further preferably less than 10 amino acids long. Portion C of the synthetic polypeptide, corresponding to the amino acid sequence between the MBD and NID amino acid sequences, may be less than 20 amino acids, preferably less than 15, 10, or 5 amino acids. Additionally or alternatively, portion C may have less than 95% identity to the amino acid sequence as depicted in SEQ ID NO: 7; preferably the identity is less than 90%, less than 85%, less than 80%, less than 75%, less than 70%, less than 65%, less than 60%, less than 50%, or less than 30%. Preferably portion C is truncated compared to the natural sequence of MeCP2, such that portion C is less than 98 amino acids long, preferably less than 90, 85, 80, 75, 70, 65, 60, 55, 50, 45, 40, 35, 30, 25, 20, or 15 amino acids long. Preferably the amino acid sequence between the MBD and NID amino acid sequences is less than 50 amino acids long, preferably less than 30 amino acids long, and further preferably less than 20 amino acids long.

An additional benefit to deletions within portion C of the synthetic polypeptide, i.e. the amino acid sequence between the MBD and NID sequences, is that the inventors have found that deletions in this portion can make the synthetic polypeptide less stable. Surprisingly, the inventors have found that such reduced stability may be beneficial to the utility of the synthetic polypeptide in the clinical setting, because it may reduce the chance of over-expression of the synthetic polypeptide. Therefore in some embodiments it is particularly preferred that there be a significant deletion in the amino acid sequence between the MBD and NID amino acid sequences, e.g. portion C of the above-noted generic structure, for example a deletion of at least 10, 15, 20, 30, 40, or 50 amino acids. Preferably the substitution or significant deletion of the amino acids will include substitution or significant deletion from the region from position 207 to position 271 of the full length human wild type MeCP2 polypeptide sequence (e2 isoform) as shown in SEQ ID NO: 4.

Portion E of the synthetic polypeptide, corresponding to the C-terminal portion of the polypeptide and the area adjacent to the carboxy end of the NID amino acid sequence when the MBD amino acid sequence is N-terminal to the NID amino acid sequence, may be absent, such that the carboxy end of the NID amino acid sequence corresponds with the C-terminus of the synthetic polypeptide. Alternatively, portion E may comprise a protein tag, for example so that portion E may be used to isolate or monitor/detect the synthetic polypeptide. Additionally or alternatively, portion E may have less than 95% identity to the amino acid sequence provided in SEQ ID NO: 8, calculated over the entire length of the amino acid sequence as depicted in SEQ ID NO: 8; preferably the identity is less than 90%, less than 85%, less than 80%, less than 75%, less than 70%, less than 65%, less than 60%, or less than 50%. Thus portion E may comprise a deletion of all or a significant part of the amino acids at positions 313 to 486 of MeCP2 (SEQ ID NO:4), optionally with replacement of the deleted sequence by a tag, and further optionally with a linker attaching the tag to the synthetic polypeptide.

Preferably the amino acid sequence adjacent to the carboxy end of the NID amino acid sequence has less than 75% identity to the amino acid sequence as depicted in SEQ ID NO: 8, calculated over the entire length of the amino acid sequence as depicted in SEQ ID NO: 8, preferably less than 50%, and further preferably less than 30% identity.

Preferably the amino acid sequence adjacent to the carboxy end of the NID amino acid sequence is less than 50 amino acids long, preferably less than 30 amino acids long or less than 20 amino acids long, and further preferably there is no amino acid sequence adjacent to the carboxy end of the NID amino acid sequence such that the carboxy end of the NID amino acid sequence corresponds with the C-terminus of the synthetic polypeptide.

The tag forming part of portion E of the synthetic polypeptides of the invention may be for monitoring or detection of the synthetic polypeptide or to allow post-translational regulation of the synthetic polypeptide, as explained further below. Examples of suitable tags for detection or monitoring of the polypeptide are known in the art, and include a polyhistidine tag, a FLAG tag, a Myc tag and a fluorescent protein tag such as enhanced green fluorescent protein (EGFP).

Preferably the synthetic polypeptides of the invention have less than 90% identity over the entire length of the amino acid sequences of MeCP2 as depicted in SEQ ID NO: 3 and SEQ ID NO: 4, preferably less than 80% identity, less than 70% identity, or less than 60% identity, and further preferably less than 40% identity.

The term “identity” refers to the extent to which two amino acid sequences have the same residues at the same positions in an alignment. The percentage identity as used herein is calculated across the length of a comparative sequence disclosed herein, for example one of SEQ ID NOs: 3 to 8, as described herein. Thus all residues in that comparative sequence should be aligned with the sequence of interest, and any gaps created during alignment of the sequence of interest with the full length of the comparative sequence should be taken into account when calculating the percentage identity, including when such “gaps” occur at either end of the sequence of interest in the alignment. However, any additional end sequence in the sequence of interest that aligns past the end of the comparative sequence, i.e. which does not align as such with the comparative sequence but which overhangs the end of the comparative sequence instead, should not be included when calculating the percentage identity. Thus when calculating the percentage identity, the identity score will be divided by the length of the comparative sequence, including any gaps that have been inserted into the comparative sequence as a result of the optimal alignment with the sequence of interest, and then multiplied by 100.

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Nat. Acad Sci. USA 85:2444, 1988; Higgins & Sharp, Gene, 73:23744, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth Mol. Bio. 24:307-31, 1994. Altschul et al., J. Mol. Biol. 215:403-10, 1990, presents a detailed consideration of sequence alignment methods and homology calculations. There are readily available programs that permit the preparation of sequence alignments and the calculation of percentage identity, such as the GAP program, running under GCG (Genetics Computer Group Inc., Madison, WI, USA).

Other variants of the synthetic polypeptides of the invention can be, for example, functional variants such salts, amides, esters, and specifically C-terminal esters, and N-acyl derivatives. Also included are peptides which are modified in vivo or in vitro, for example by glycosylation, amidation, carboxylation or phosphorylation.

A benefit of the invention is that by identifying the MBD and NID sequences as being key to the activity of MeCP2 that is deficient in Rett syndrome, the inventors have identified that significant portions of other sections of the MeCP2 protein may be removed when preparing a synthetic protein for use in treating or preventing a disorder such as Rett syndrome. Therefore the invention provides synthetic polypeptides that are truncated forms of MeCP2, comprising MBD and NID sequences but missing one or more sections of the amino acid sequences adjacent to the amino end of the MBD amino acid sequence (e.g. in portion A), between the MBD and NID amino acid sequences (e.g. in portion C), and adjacent to the carboxy end of the NID amino acid sequence (e.g. in portion E), when compared to the full length human MeCP2 e1 and e2 sequences (SEQ ID NOs 3 and 4). Preferably a synthetic polypeptide of the invention will have less amino acids than the wild type MeCP2 protein, for example a synthetic polypeptide of the invention may consist of less than 430 amino acids, preferably less than 400, 350, 320, 270, or 200 amino acids, and further preferably less than 180 amino acids.

Synthetic polypeptides of the invention may suitably comprise a signal peptide, for example a nuclear localisation signal. A nuclear localisation signal may target a polypeptide for import into the cell nucleus by nuclear transport. Suitable nuclear localisation signals are known in the art, and include the SV40 nuclear localisation signal and the NLS of the native MeCP2 protein (amino acids 253 to 271 of SEQ ID NO: 4). The NLS may be situated in any part of the polypeptide, but preferably will be situated in the amino acid sequence linking the MBD to the NID.

Synthetic polypeptides of the invention may suitably comprise a cell-penetrating peptide (CPP). CPPs, also called protein-transduction domains, consist of short sequences of 8 to 30 amino acids in length that can facilitate entry of molecules into cells¹⁵. The CPP may be synthetic, designed specifically for the targeting of therapeutic molecules, or naturally occurring. Preferably the CPP will facilitate entry into neuronal cells. A preferred CPP for use with the synthetic polypeptides of the invention is the CPP of the trans-activator of transcription protein, Tat.

Synthetic polypeptides of the invention may suitably comprise a tag. Such tags are well known in the art and may be useful for polypeptide purification, detection/monitoring and/or post-translational regulation. Examples of suitable tags useful in purification or detection of a polypeptide are known in the art, and include a polyhistidine tag, a FLAG tag, a Myc tag and a fluorescent protein tag such as enhanced green fluorescent protein (EGFP).

A tag may be used for post-translational regulation of a polypeptide by, for example, providing the ability to control the post-translational degradation of the polypeptide. Examples of suitable tags that may be used with synthetic polypeptides of the invention to allow such control of post-translational degradation include a SMASh tag and a Destabilisation Domain (DD) of FKBP12. The SMASh tag is approximately 300 amino acids long and comprises a protease cleavage site followed by the protease, followed by a degron tag. The SMASh tag is fused in the protein of interest (POI) with the protease cleavage site and protease between the POI and the degron tag. This can be on either terminus of the POI. Normally, the protease self-cleaves the protease site, removing the degron tag so that the protein is not degraded. However, treatment with a drug such as Asunaprevir can inhibit the protease, which prevents removal of the degron tag, and so results in degradation of the attached POI. Since the administration of Asunaprevir has a dose-dependent effect on POI degradation, the use of a SMASh tag with Asunaprevir can allow post-translational regulation of the amount of the POI.

Similarly, the DD-FKBP12 is approximately 110 amino acids long and it destablises the POI to which it is attached. It can be fused to either terminus of the POI, but it is preferably attached to the N-terminus as then it is generally more effective. The fusion protein produced will therefore generally be unstable but it can be protected by the administration of a molecule called Shield-1. Administration of Shield-1 has a dose-dependent effect on the prevention of the degradation of the POI.

The skilled person will appreciate that it may not be desirable to include certain types of tags, particularly those used for purification or detection during polypeptide synthesis such as Myc or EGFP, in the final therapeutic polypeptide that is delivered to a subject, as the tags may be immunogenic or active in an undesirable manner. Therefore the tags included in the synthetic polypeptides of the invention may be removable, for example by chemical agents or enzymatic means, such as proteolysis or intein splicing; this may allow the use of the tag during preparation of a synthetic polypeptide followed by removal of the tag before the polypeptide is introduced to an animal for treatments in accordance with therapeutic uses (e.g. protein replacement therapy) disclosed herein.

The synthetic polypeptides of the invention may comprise a linker sequence, for example to link the MBD sequence to the NID sequence, in place of the natural sequence that links these two sequences in MeCP2, or as a means to attach or insert a tag, CPP or NLS to a synthetic polypeptide of the invention. The design and use of such linkers are well known in the art. A suitable linker may comprise between 4 and 15 amino acids, preferably between 6 and 10 amino acids. Preferably the linker will consist of glycines, serines, or a combination of glycines and serines.

Preferably the synthetic polypeptide sequences of the invention will show at least 80% similarity to one or more of the sequences ΔNC (SEQ ID NO: 13), ΔNIC (SEQ ID NO: 14), ΔN mouse (SEQ ID NO: 15), ΔNC mouse (SEQ ID NO: 16), and ΔNIC mouse (SEQ ID NO: 17), preferably at least 85%, 90%, 91%, 92%, 93%, 94%, 95%, 96%, 97%, 98%, or 99% similarity. Optionally, as disclosed above, the synthetic polypeptide sequences of the invention may comprise an e1 or e2 specific sequence, preferably at the N-terminus of the synthetic polypeptide. Thus preferably the synthetic polypeptide sequences of the invention will show at least 80% similarity to a sequence consisting of one or more of the sequences: ΔNC (SEQ ID NO: 13); ΔNIC (SEQ ID NO: 14); ΔN mouse (SEQ ID NO: 15); ΔNC mouse (SEQ ID NO: 16); and ΔNIC mouse (SEQ ID NO: 17), and one of the e1 or e2 specific sequences (SEQ ID NOs: 9-12) at the N-terminus of the sequence, and preferably at least 85%, 90%, 91%, 92%, 93%, 94%, 95%, 96%, 97%, 98%, or 99% similarity. Further preferably the synthetic polypeptide sequences of the invention may comprise or consist of the sequence ΔNC (SEQ ID NO: 13), ΔNIC (SEQ ID NO: 14), ΔN mouse (SEQ ID NO: 15), ΔNC mouse (SEQ ID NO: 16), ΔNIC mouse (SEQ ID NO: 17), or one of the sequences ΔNC (SEQ ID NO: 13), ΔNIC (SEQ ID NO: 14), ΔN mouse (SEQ ID NO: 15), ΔNC mouse (SEQ ID NO: 16), ΔNIC mouse (SEQ ID NO: 17), with one of the e1 or e2 specific sequences (SEQ ID NOs: 9-12) at the N-terminus of the sequence. ΔNC (SEQ ID NO: 13), ΔN mouse (SEQ ID NO: 15), and ΔNC mouse (SEQ ID NO: 16) include the wild type MeCP2 NLS sequence. ΔNIC (SEQ ID NO: 14) and ΔNIC mouse (SEQ ID NO: 17) include the SV40 NLS sequence.

It is particularly preferred that the synthetic polypeptide sequences of the invention will show at least 80% similarity to a sequence consisting of ΔNIC (SEQ ID NO: 14) and a human e1 or e2 specific sequence (SEQ ID NOs: 11 and 12) immediately adjacent the N-terminus of ΔNIC (SEQ ID NO: 14), preferably at least 85%, 90%, 91%, 92%, 93%, 94%, 95%, 96%, 97%, 98%, or 99% similarity. Further preferably the synthetic polypeptide sequences of the invention may comprise or consist of ΔNIC (SEQ ID NO: 14), optionally with a human e1 or e2 specific sequence (SEQ ID NOs: 11 and 12) immediately adjacent the N-terminus of ΔNIC (SEQ ID NO: 14). Synthetic polypeptides of the invention may be used in the treatment or prevention of a neurological disorder associated with inactivating mutation of MECP2, for example Rett syndrome, as explained further below.

### Nucleic Acid Constructs, Expression Vectors, Virions and Cells

The invention provides nucleic acid constructs encoding a synthetic polypeptide of the invention, and expression vectors comprising a nucleotide sequence encoding a synthetic polypeptide of the invention. The expression vector may be a viral vector, and thus the invention also provides a virion comprising an expression vector according to the invention. The invention also provides cells that comprise a synthetic genetic construct adapted to express a polypeptide of the invention, cells comprising a vector of the invention, and cells for producing a virion of the invention.

Nucleic acid constructs and/or expression vectors suitably comprise at least one expression control sequence operably linked to a nucleotide sequence encoding a synthetic polypeptide of the invention, to drive expression of the synthetic polypeptide. “Expression control sequences” are nucleotide sequences located upstream (5′ non-coding sequences), within, or downstream (3′ non-coding sequences) of a coding sequence, and which influence the transcription, RNA processing or stability, or translation of the associated coding sequence. Expression control sequences include enhancers, promoters, translation leader sequences, introns, and polyadenylation signal sequences. They include natural and synthetic sequences as well as sequences that may be a combination of synthetic and natural sequences. As is noted herein, the term “expression control sequences” is not limited to promoters. However, some suitable expression control sequences useful in the present invention will include, but are not limited to constitutive promoters, tissue-specific promoters, development-specific promoters, regulatable promoters and viral promoters.

Such expression control sequences generally comprise a promoter sequence and additional sequences which regulate transcription and translation and/or enhance expression levels. Suitable expression control sequences are well known in the art and include eukaryotic, prokaryotic, or viral promoter or poly-A signal. Expression control and other sequences will, of course, vary depending on the host cell selected or can be made inducible. Examples of useful promoters are the SV-40 promoter (Science 1983, 222: 524-527), the metallothionein promoter (Nature 1982, 296: 39-42), the heat shock promoter (Voellmy et al., P.N.A.S. USA 1985, 82: 4949-4953), the PRV gX promoter (Mettenleiter and Rauh, J. Virol. Methods 1990, 30: 55-66), the human CMV IE promoter (U.S. Pat. No. 5,168,062), the Rous Sarcoma virus LTR promoter (Gorman et al., P.N.A.S. USA 1982, 79: 6777-6781), or human elongation factor 1 alpha or ubiquitin promoter. Suitable control sequences to drive expression in animals, e.g. humans, are well known in the art. The expression control sequences can drive ubiquitous expression or tissue- or cell-specific expression. The expression control sequence can comprise, for example, a viral or human promoter. A suitable promoter can be ubiquitous (e.g. the CAG promoter), tissue restricted or tissue specific. For example, the NESTIN promoter may drive expression in the CNS and the TAU and SYNAPSIN promoters may drive expression in neurons. Preferably the promoter will be for expression of the nucleotide sequence in neuronal cells, for example the MECP2 or Mecp2 promoter. Many suitable control sequences are known in the art, and it would be routine for the skilled person to select suitable sequences for the expression system being used.

The expression of the MECP2 is tightly controlled in animals. Therefore where nucleic acid constructs and expression vectors of the invention are to be used for expression of the synthetic polypeptides of the invention in an animal, for example in gene therapy, it is preferred that said expression be specific to neural cells, particularly neural cells of the brain and CNS. This may be accomplished, for example, through specific targeting of the nucleic acid constructs and expression vectors to the brain and/or the neural cells, through the use of delivery vehicles, such as AAV virions, and/or specific administration routes, such as by administration directly to the CNS. Additionally or alternatively, said neuron specific expression may be accomplished by the use of expression control sequences in the nucleic acid constructs and/or expression vectors that substantially limit the expression of the synthetic polypeptide to neural cells. Preferably those expression control sequences will be selected from the expression control sequences used to control the natural expression of the MECP2 gene.

The MECP2 gene contains a remarkably large, highly conserved 3′UTR, in which enhancers, silencers and many miRNA binding sites have been identified. Similarly, the MECP2 gene promoter region is also very large, and includes silencer, regulatory element and promoter sequences. Therefore preferably the expression vectors and/or nucleic acid constructs of the invention will include one or more of the expression control sequence elements from the MECP2 3′UTR. For example, gene therapy is disclosed herein that used an expression cassette that included an upstream core promoter element from the Mecp2 gene, and downstream microRNA (miR) binding sites and an AU-rich element. Therefore an expression vector and/or nucleic acid construct of the invention will preferably comprise one or more elements selected from: an upstream MECP2 or Mecp2 core promoter sequence (see, for example, nucleotides 200 to 329, inclusive, of SEQ ID NO:65); one or more downstream miR binding sites from the MECP2 or Mecp2 3′UTR; and an AU-rich element from the MECP2 or Mecp2 3′UTR. Further preferably the one or more downstream miR binding sites from the MECP2 or Mecp23′UTR will comprise one or more, or all of the following miR binding sites: a binding site for mir-22 (nucleotides 1166 to 1195, inclusive, of SEQ ID NO:65), a binding site for mir-19 (nucleotides 1196 to 1224, inclusive, of SEQ ID NO:65), a binding site for mir-132 (nucleotides 1225 to 1252, inclusive, of SEQ ID NO:65), and a binding site for mir-124 (nucleotides 1318 to 1324, inclusive, of SEQ ID NO:65).

Preferably, the expression vector and/or nucleic acid construct of the invention may comprise an upstream CNS regulatory element from Mecp2 or MECP2 (see, for example, nucleotides 422 to 443, inclusive, of SEQ ID NO:65) and/or an upstream silencer from Mecp2 or MECP2 (see, for example, nucleotides 142 to 203, inclusive, of SEQ ID NO:65).

It is particularly preferred that the upstream region of the expression vector and/or nucleic acid construct of the invention will comprise the mMeP426 sequence (nucleotides 117 to 542 of SEQ ID NO: 65; see FIG. 17B, C) and/or that the downstream region of the expression vector and/or nucleic acid construct of the invention will comprise the RDH1 pA sequence (nucleotides 1166 to 1370 of SEQ ID NO: 65; see FIG. 17B, C).

Of course, the skilled person will appreciate that one or more other sequence elements may also be desirable or required in an expression vector and/or nucleic acid construct of the invention, such as a translational initiation signal, e.g. a Kozak sequence, a polyadenylation signal, and binding sites for components of the polyadenylation machinery such as CstF (cleavage stimulation factor). The skilled person will be capable of designing an expression vector and/or nucleic acid construct in accordance with the invention having any and all such necessary or desirable well known sequences.

The human wild type MECP2 e1 isoform cDNA sequence is provided herein as SEQ ID NO: 18. The mouse wild type MECP2 e1 isoform cDNA sequence is provided herein as SEQ ID NO: 21.

Suitably the nucleic acid construct of the invention may comprise a sequence for encoding the cDNA sequence of ΔNC (SEQ ID NO: 19), ΔNIC (SEQ ID NO: 20), ΔN mouse (SEQ ID NO: 22), ΔNC mouse (SEQ ID NO: 23), or ΔNIC mouse (SEQ ID NO: 24). As explained above, optionally the synthetic polypeptides of the invention may comprise the e1 or e2 specific N-terminal sequences. The mouse e1 specific N-terminal amino acid sequence is encoded by the cDNA sequence ATGGCCGCCGCTGCCGCCACCGCCGCCGCCGCCGCCGCGCCGAGCGGAGGAGGAG GAGGAGGCGAGGAGGAGAGACTGGAGGAAAAG (SEQ ID NO: 25). The mouse e2 specific N-terminal amino acid sequence is encoded by the cDNA sequence ATGGTAGCTGGGATGTTAGGGCTCAGGGAGGAAAAGGGAGGAAAAG (SEQ ID NO: 26). The human e1 specific N-terminal amino acid sequence is encoded by the cDNA sequence ATGGCCGCCGCCGCCGCCGCCGCGCCGAGCGGAGGAGGAGGAGGAGGCGAGGAGG AGAGACTGGAAGAAAAG (SEQ ID NO: 27). The human e2 specific N-terminal amino acid sequence is encoded by the cDNA sequence ATGGTAGCTGGGATGTTAGGGCTCAGGGAAGAAAAG (SEQ ID NO: 28). Therefore the nucleic acid construct of the invention may comprise a sequence for encoding the cDNA sequence according to any of SEQ ID NOs: 25-28.

Further preferably the nucleic acid construct of the invention may comprise a sequence for encoding the cDNA sequence of SEQ ID NO: 28 or 29 immediately adjacent to the cDNA sequence of ΔNIC (SEQ ID NO: 20).

Due to the degeneracy of the genetic code, polynucleotides encoding an identical or substantially identical amino acid sequence may utilise different specific codons (e.g. synonymous base substitutions). All polynucleotides encoding the synthetic polypeptides as defined above are considered to be part of the invention.

The invention provides an expression vector comprising a nucleotide sequence encoding a synthetic polypeptide of the invention. Such vectors suitably comprise an isolated or synthetic nucleic acid construct as described above.

The vectors according to the invention are suitable for transforming a host cell. Examples of suitable cloning vectors are plasmid vectors such as pBR322, the various pUC, pEMBL and Bluescript plasmids, or viral vectors such as HVT (Herpes Virus of Turkeys), MDV (Marek Disease Virus), ILT (Infectious Laryngotracheitis Virus), FAV (Fowl Adenovirus), FPV (FowlpoxVirus), or NDV (Newcastle Disease Virus). pcDNA3.1 is a particularly preferred vector for expression in animal cells.

After the polynucleotide has been cloned into an appropriate vector, the construct may be transferred into the cell, bacteria, or yeast by means of an appropriate method, such as electroporation, CaCl2 transfection or lipofectins. When a baculovirus expression system is used, the transfer vector containing the polynucleotide may be transfected together with a complete baculo genome.

These techniques are well known in the art and the manufacturers of molecular biological materials (such as Clontech, Stratagene, Promega, and/or Invitrogen) provide suitable reagents and instructions on how to use them. Furthermore, there are a number of standard reference text books providing further information on this, e.g. Rodriguez, R. L. and D. T. Denhardt, ed., “Vectors: A survey of molecular cloning vectors and their uses”, Butterworths, 1988; Current protocols in Molecular Biology, eds.: F. M. Ausubel et al., Wiley N. Y., 1995; Molecular Cloning: a laboratory manual, supra; and DNA Cloning, Vol. 1-4, 2nd edition 1995, eds.: Glover and Hames, Oxford University Press).

Details of preferred proteins according to the present invention for expression via the vector are described above.

The vector may be adapted to provide transient expression in a host cell or stable expression. Stable expression can be achieved, for example, through integration of the nucleotide sequence encoding the synthetic polypeptide into the genome of the host cell.

Suitable viral vectors include retroviral vectors (including lentiviral vectors), adenoviral vectors, adeno-associated viral (AAV) vectors, and alphaviral vectors. Preferably the viral vector will be an AAV vector, such as AAV1, AAV2, AAV4, AAV5, AAV6, AAV8 or AAV9. Preferably the AAV vector will be a self-complementary (sc) AAV vector.

The vector of the present invention may be present in a virion. Thus the present invention also provides a virion comprising a vector in accordance with the present invention. Preferably the virion and/or viral vector will be for expression in cells of the central nervous system (CNS), such as neuronal cells. Thus preferably a virion of the invention will comprise a capsid and/or inverted terminal repeats (ITRs) from one or more of AAV1, AAV2, AAV4, AAV5, AAV6, AAV8, and AAV9. Preferably the AAV will be a self-complementary (sc) AAV vector. Further preferably, the ITR and capsid proteins may be from different serotypes, for example ITRs from AAV2 may be used with capsid proteins from AAV9 to form scAAV virions.

Vectors according to the present invention can be used in transforming cells for expression of a protein according to the present invention. This can be done in cell culture to produce recombinant protein for harvesting, or it can be done in vivo to deliver a protein according to the present invention to an animal.

Thus the present invention also provides a cell population in which cells comprise a synthetic genetic construct adapted to express a protein according to the present invention. Said cell population may be present in a cell-culture system in a suitable medium to support cell growth.

The cells can be eukaryotic or prokaryotic.

Polynucleotides of the present invention may be cloned into any appropriate expression system. Suitable expression systems include bacterial expression system (e.g. Escherichia coli DH5a), a viral expression system (e.g. Baculovirus), a yeast system (e.g. Saccharomyces cerevisiae) or eukaryotic cells (e.g. COS-7, CHO, BHK, HeLa, HD11, DT40, CEF, or HEK-293T cells). A wide range of suitable expression systems are available commercially. Typically the polynucleotide is cloned into an appropriate vector under control of a suitable constitutive or inducible promoter and then introduced into the host cell for expression.

Suitably the cells are animal cells, more preferably they are mammalian cells, and most preferably human cells. Suitably the cells comprise a vector as set out above.

Preferably the cells are adapted such that expression of the protein according to the present invention is inducible.

It is particularly preferred that the cells comprise an expression vector for expressing a synthetic polypeptide of the invention, and the cell is suitable or adapted for producing a virion comprising an expression vector of the invention. Thus the cells may be used to produce virions for use in gene therapy treatment of Rett syndrome and related disorders. Preferably the virions will comprise AAV vectors for expressing a polypeptide of the invention, and further preferably the AAV vectors will comprise AAV9 and/or AAV2.

Suitable host cells for producing AAV virions include microorganisms, yeast cells, insect cells, and mammalian cells, that can be, or have been, used as recipients of a heterologous DNA molecule. The term includes the progeny of the original cell which has been transfected. Thus, a “host cell” as used herein generally refers to a cell which has been transfected with an exogenous DNA sequence. Cells from the stable human cell line, 293 (readily available through, e.g., the American Type Culture Collection under Accession Number ATCC CRL1573) can be used in the practice of the present invention. Particularly, the human cell line 293 is a human embryonic kidney cell line that has been transformed with adenovirus type-5 DNA fragments, and expresses the adenoviral Ela and Elb genes. The 293 cell line is readily transfected, and provides a particularly convenient platform in which to produce AAV virions.

Suitably, for in vivo delivery, virions of the invention, such as AAV virions, may be formulated into pharmaceutical compositions. Suitably, pharmaceutical compositions will comprise sufficient genetic material to produce a therapeutically effective amount of the synthetic polypeptide of the invention, i.e., an amount sufficient to reduce, ameliorate or prevent symptoms of the disorders associated with reduced MeCP2 activity, such as Rett syndrome. The pharmaceutical compositions may also contain a pharmaceutically acceptable excipient. Such excipients include any pharmaceutical agent that does not itself induce the production of antibodies harmful to the individual receiving the composition, and which may be administered without undue toxicity. Pharmaceutically acceptable excipients include, but are not limited to, sorbitol, Tween80, and liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles.

As is apparent to those skilled in the art, an effective amount of viral vector which must be added can be empirically determined. Administration can be effected in one dose, continuously or intermittently throughout the course of treatment. Methods of determining the most effective means and dosages of administration are well known to those of skill in the art and will vary with the viral vector, the composition of the therapy, and the subject being treated. Single and multiple administrations can be carried out with the dose level and pattern being selected by the treating physician.

Pharmaceutical Compositions, Methods of Prevention and Treatment, and Use in Same

The synthetic polypeptides of the invention are useful for replacing defective MeCP2 in the cells of those affected by Rett syndrome or related disorders. Thus the invention provides a method of treating or preventing disease in an animal comprising administering a synthetic polypeptide of the invention. Preferably the disease is a neurological disorder associated with inactivating mutation of MECP2, for example Rett syndrome. Preferably the animal is a human patient.

The administering in the methods of the invention may comprise administering a composition comprising a synthetic polypeptide of the invention, administering an expression vector of the invention, and/or administering a virion of the invention.

The invention therefore also provides synthetic polypeptides, expression vectors and virions for the treatment or prevention of a neurological disorder associated with inactivating mutation of MECP2, for example Rett syndrome, and the use of a synthetic polypeptide of the invention, an expression vector of the invention, or a virion of the invention, in the manufacture of a medicament for the treatment or prevention of a neurological disorder associated with inactivating mutation of MECP2, for example Rett syndrome.

The disorders that may be treated or prevented as provided herein include those involving a reduction or inactivation in the activity of MeCP2. Thus, as used herein, the phrase “inactivating mutation” encompasses mutations that result in reduced MeCP2 activity as well as mutations that abolish MeCP2 activity, and in particular the activity due to the ability of MeCP2 to recruit members of the NCoR/SMRT co-repressor complex to methylated DNA. Such disorders may be recognised, for example, by the identification of mutations in MeCP2 in the subject having, or at risk of having, the disorder. For example, recurrent (e.g. A140V) or sporadic mutations in males have been found to be causative in some cases of X-linked intellectual disability. Similarly, in females, “hypomorphic” mutations of this kind are associated with learning disability, and exome sequencing of children diagnosed with developmental delay is also revealing mutations in MECP2. Such mutations may affect the ability of the MeCP2 to recruit components of the NCoR/SMRT co-repressor complex to methylated DNA, and/or may generally affect the stability of the protein.

In the context of the methods and medical uses of the present invention, the animal to be treated may be anyone requiring the treatment, or anyone deemed to be at risk of developing a relevant disorder. Suitably the animal may be a mammal, preferably a primate and further preferably a human subject.

The animal to be treated may present with symptoms suggestive of a MeCP2 associated disorder. Alternatively, the subject may appear to be asymptomatic but deemed to be at risk of developing an MECP2-related disorder caused by loss of MeCP2 function, such that preventative treatment with synthetic polypeptides of the invention is desirable. Suitably an asymptomatic subject may be a subject who is believed to be at elevated risk of having a MeCP2-associated disorder. Such an asymptomatic subject may be one who has a family history of MeCP2, or one who has undergone genetic testing that indicates a mutation in the MECP2 gene.

The present invention envisions treating or preventing disorders associated with reduced MeCP2 activity by the administration of a therapeutic agent, i.e., a synthetic polypeptide composition, a nucleic acid construct, an expression vector, and/or a virion of the invention. Administration of the therapeutic agents in accordance with the present invention may be continuous or intermittent, depending, for example, upon the recipient's physiological condition, whether the purpose of the administration is therapeutic or prophylactic, and other factors known to skilled practitioners. The administration of the agents of the invention may be essentially continuous over a preselected period of time or may be in a series of spaced doses. Both local and systemic administration is contemplated.

One or more suitable unit dosage forms having the therapeutic agent(s) of the invention can be administered by a variety of routes including parenteral, including by intravenous and intramuscular routes, as well as by direct injection into the tissue directly associated with the reduced MeCP2 activity. For example, the therapeutic agent may be directly injected into the brain. Alternatively the therapeutic agent may be introduced intrathecally for brain and spinal cord conditions. In another example, the therapeutic agent may be introduced intramuscularly for viruses that traffic back to affected neurons from muscle, such as AAV, lentivirus and adenovirus. The formulations may, where appropriate, be conveniently presented in discrete unit dosage forms and may be prepared by any of the methods well known to pharmacy. Such methods may include the step of bringing into association the therapeutic agent with liquid carriers, solid matrices, semi-solid carriers, finely divided solid carriers or combinations thereof, and then, if necessary, introducing or shaping the product into the desired delivery system.

When the therapeutic agents of the invention are prepared for administration, they are preferably combined with a pharmaceutically acceptable carrier, diluent or excipient to form a pharmaceutical formulation, or unit dosage form. The total active ingredients in such formulations include from 0.1 to 99.9% by weight of the formulation. A “pharmaceutically acceptable carrier” is a diluent, excipient, and/or salt that is compatible with the other ingredients of the formulation, and not deleterious to the recipient thereof. The active ingredient for administration may be present as a powder or as granules, as a solution, a suspension or an emulsion.

Pharmaceutical formulations containing the therapeutic agents of the invention can be prepared by procedures known in the art using well known and readily available ingredients.

The therapeutic agents of the invention can also be formulated as solutions appropriate for parenteral administration, for instance by intramuscular, subcutaneous or intravenous routes. The pharmaceutical formulations of the therapeutic agents of the invention can also take the form of an aqueous or anhydrous solution or dispersion, or alternatively the form of an emulsion or suspension.

Thus, the therapeutic agent may be formulated for parenteral administration (e.g., by injection, for example, bolus injection or continuous infusion) and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

The pharmaceutical formulations of the present invention may include, as optional ingredients, pharmaceutically acceptable carriers, diluents, solubilizing or emulsifying agents, and salts of the type that are well-known in the art. Specific non-limiting examples of the carriers and/or diluents that are useful in the pharmaceutical formulations of the present invention include water and physiologically acceptable buffered saline solutions such as phosphate buffered saline solutions pH 7.0-8.0 saline solutions and water.

### BRIEF DESCRIPTION OF THE FIGURES

The invention will now be described in detail with reference to a specific embodiment and with reference to the accompanying drawings, in which:

FIG. 1 shows stepwise deletion of MeCP2 protein, retaining only the two key functional domains predicted by mutational analysis.
A) Schematic of human MeCP2 protein sequence (e1 isoform) with the methyl-CpG binding domain (MBD) [residues 78-162¹²] and the NCoR/SMRT interaction domain (NID) [residues 285-3094]; annotated with (above): polymorphisms in healthy hemizygous males, RTT-causing missense mutations, and (below) sequence identity to chimpanzee (e1), mouse (e1), Xenopus and zebrafish homologues [sites of insertions in Xenopus and zebrafish sequences shown as longer bars].
B) Schematic of the deletions series of MeCP2 proteins (mouse e2 isoforms) expressed by the three novel mouse lines presented in this study (and WT-EGFP mice¹⁶)
C) EGFP-tagged shortened proteins were overexpressed in HeLa cells, and immunoprecipitated using GFP-TRAP beads. Western blots show expression and purification of these protein (GFP), and co-immunoprecipiation of NCoR/SMRT co-repressor complex components (NCoR, HDAC3 and TBL1XR1). WT-EGFP and R306C-EGFP were used as controls to show the presence and absence of binding to these proteins, respectively. ‘In’=input, ‘IP’=immunoprecipiate.
D) EGFP-tagged shortened MeCP2 proteins were overexpressed in NIH-3T3 cells, which were PFA fixed and stained with DAPI. WT-EGFP and R111G-EGFP were used as controls to show focal and diffuse localisation, respectively.
E) EGFP-tagged shortened proteins were co-overexpressed with TBL1X-mCherry in NIH-3T3 cells, which were PFA fixed and stained with DAPI. WT-EGFP and R306C-EGFP were used as controls to show the presence and absence TBL1X-mCherry recruitment to heterochromatic foci, respectively.

FIG. 2 shows the design of the MeCP2 deletion series.
A) Schematic of the genomic DNA sequences of wild-type and ΔNIC MeCP2, showing the retention of the extreme N-terminal amino acids encoded in exons 1 and 2 and the first 10 bp of exon 3, the deletion of the N- and C-termini, the replacement of the intervening region with a linker and SV40 NLS, and the addition of the C-terminal EGFP tag. Colour key: 5′UTR=white, MBD=mid-grey, NID=dark grey, uncharacterised regions=grey, SV40 NLS=mid-grey beside linker, linkers=dark grey and EGFP=C-terminal mid-grey.
B) The N-terminal ends of the sequences of all three shortened proteins (e1 and e2 isoforms) showing the fusion of the extreme N-terminal amino acids to the MBD (starting with Pro72).
C), D) Protein sequence alignment of the (C) MBD and (D) NID region using ClustalWS, shaded according to BLOSUM62 score. Both alignments are annotated with: (above) RTT-missense mutations¹³ and activity-dependent phosphorylation sites^{17,18,19}; and (below) sequence conservation, interaction domains and known²⁰/predicted²¹ structure. Interaction sites: meDNA binding (residues 78-162¹²), AT hook 1 (residues 183-195²²), AT hook 2 (residues 257-272²³), NCoR/SMRT binding (residues 285-309⁴). The bipartite nuclear localisation signal (NLS) is also shown (residues 253-256 and 266-271). Residue numbers correspond to that of mammalian e2 isoforms. The regions retained in ΔNIC are: MBD resides 72-173 (highlighted by the grey rectangle in C) and NID resides 272-312 (highlighted by the grey rectangle in D).

FIG. 3 shows the constructs for the generation of ΔN and ΔNC mice.

(Upper) Diagram of (A) ΔN and (B) ΔNC mouse production. The endogenous Mecp2 allele was targeted in male ES cells. The selection cassette was removed in vivo by crossing chimaeras with deleter (CMV-Cre) mice.

(Lower) Southern blot analysis shows correct targeting of ES cells and successful cassette deletion in the knock-in mice.

FIG. 4 shows constructs for the generation of ΔNIC and STOP mice.

(Upper) Diagram of ΔNIC mouse production. The endogenous Mecp2 allele was targeted in male ES cells. The selection cassette was removed in vivo by crossing chimaeras with deleter (CMV-Cre) mice to produce constitutively expressing ΔNIC mice or retained to produce STOP mice.

(Lower) Southern blot analysis shows correct targeting of ES cells and successful cassette deletion in the ΔNIC knock-in mice.

FIG. 5 shows that ΔN and ΔNC proteins are expressed at around wild-type levels in knock-in mice.

A, Upper) Western blot analysis of crude whole brain extract showing protein sizes and levels in ΔN mice (n=3) compared to their wild-type littermates (n=3), detected using a C-terminal MeCP2 antibody.

A, Lower) Western blot analysis of ΔN (n=3) and ΔNC (n=3) mice compared to WT-EGFP controls (n=3), detected using a GFP antibody. Histone H3 was used as a loading control. *denotes a non-specific band detected by the GFP antibody.
B) Flow cytometry analysis of protein levels in nuclei prepared from whole brain (‘All’) and the high-NeuN subpopulation (‘Neurons’) in WT-EGFP (n=3), ΔN (n=3) and ΔNC (n=3) mice, detected using EGFP fluorescence. Graph shows mean±S.E.M and genotypes were compared to WT-EGFP controls by t-test: All ΔN p=0.338, ΔNC **p=0.003; and Neurons ΔN p=0.672, ΔNC *p=0.014.

FIG. 6 shows deletion of the N- and C-termini has minimal phenotypic consequence.
A), B) Phenotypic scoring of hemizygous male (A) ΔN mice (n=10) and (B) ΔNC mice (n=10) each compared to their wild-type littermates (n=10) over one year. Graphs show mean scores±S.E.M. Mecp2-null data (n=12)¹⁶ is used as a comparator.
C), D) Kaplan-Meier plots showing survival of the same cohorts in parts A and B. Mecp2-null data (n=24)¹⁶ is used as a comparator.
E), F), G) Behavioural analysis of separate cohorts performed at 20 weeks of age: ΔN (n=10) and ΔNC mice (n=10-11) each compared to their wildtype littermates (n=10). All graphs show individual values and medians, and the results of statistical analysis comparing genotypes (see below): not significant (‘n.s.’) p>0.05, *p<0.05.
E) Time spent in the closed and open arms of the Elevated Plus Maze was measured during a 15 minute trial, and genotypes were compared using KS tests: ΔN cohort (left) closed arms p=0.988 and open arms p=0.759; ΔNC cohort (right) closed arms p=0.956 and open arms p=0.932.
F) Time spent in the centre region of the Open Field test was measured during a 20 minute trial, and genotypes were compared using t-tests: ΔN p=0.822; ΔNC *p=0.020.
G) Average latency to fall from the Accelerating Rotarod in four trials was calculated for each of the three days of the experiment, and genotypes were compared using KS tests: ΔN cohort day 1 p=0.759, day 2 p=0.401 and day 3 p=0.055; ΔNC cohort day 1 p=0.988, day 2 p=0.401 and day 3 p=0.759.

FIG. 7 shows that ΔNC have a slightly increased weight phenotype that is background-dependent.
A), B) Growth curves of the backcrossed scoring cohorts (see FIG. 6A-D).
C) Growth curve of an outbred (75% C57BL/6J) cohort of ΔNC mice (n=7) and wild-type littermates (n=9).
A), B), C) Graphs show mean values±S.E.M. Genotypes were compared using repeated measures ΔNOVA: ΔN p=0.385, ΔNC ****p<0.0001, ΔNC (outbred) p=0.739. Mecp2-null data (n=20)¹⁶ is used as a comparator.

FIG. 8 shows that no activity phenotype was detected for either ΔN or ΔNC mice.
A), B) Behavioural analysis of ΔN (n=10) and ΔNC mice (n=10) each compared to their wildtype littermates (n=10) at 20 weeks of age (see FIG. 6E-G). Total distance travelled the Open Field test was measured during a 20 minute trial. Graphs show individual values and medians, and genotypes were compared using t-tests: ΔN p=0.691; ΔNC p=0.791.

FIG. 9 shows that additional deletion of the intervening region leads to protein instability and mild RTT-like symptoms.
A) Western blot analysis of crude whole brain extract showing protein sizes and levels in ΔNIC mice (n=3) and WT-EGFP controls (n=3), detected using a GFP antibody. Histone H3 was used as a loading control. *denotes a non-specific band detected by the GFP antibody.
B) Flow cytometry analysis of protein levels in nuclei prepared from whole brain (‘All’) and the high-NeuN subpopulation (‘Neurons’) in ΔNIC mice (n=3) and WT-EGFP controls (n=3), detected using EGFP fluorescence. Graph shows mean±S.E.M and genotypes were compared by t-test: All ***p=0.0002 and Neurons ***p=0.0001.
C) Quantitative PCR analysis of mRNA levels prepared from whole brain of ΔNIC mice (n=3) and wild-type littermates (n=3). Mecp2 transcript levels were normalised to Cyclophilin A. Graph shows mean±S.E.M (relative to wild-type) and genotypes were compared by t-test: **p=0.005.
D) Phenotypic scoring of ΔNIC mice (n=10) compared to their wild-type littermates (n=10) over one year. Graph shows mean scores±S.E.M. Mecp2-null data (n=12)¹⁶ is used as a comparator.
E) Kaplan-Meier plot showing survival of the same cohort in part D. One ΔNIC animal died at 43 weeks without exceeding a severity score of 2.5. Mecp2-null data (n=24)¹⁶ is used as a comparator.
F), G), H) Behavioural analysis of separate cohorts performed at 20 weeks of age: ΔNIC (n=10) compared to their wildtype littermates (n=10). All graphs show individual values and medians, and the results of statistical analysis comparing genotypes (see below): not significant (‘n.s.’) p>0.05, *p<0.05, **p<0.01.
F) Time spent in the closed and open arms and central region of the Elevated Plus Maze was measured during a 15 minute trial, and genotypes were compared using KS tests: closed arms **p=0.003, open arms p=0.055 and centre *p=0.015.
G) Time spent in the centre region of the Open Field test was measured during a 20 minute trial, and genotypes were compared using a t-test: p=0.402.
H) Average latency to fall from the Accelerating Rotarod in four trials was calculated for each of the three days of the experiment, and genotypes were compared using KS tests: day 1 p=0.164, day 2 p=0.055 and day 3 **p=0.003. Changed performance (learning/worsening) over the three day period was determined using Friedman tests: wild-type animals p=0.601, ΔNIC animals **p=0.003.

FIG. 10 shows that outbred ΔNIC mice had 100% survival over one year.

Kaplan-Meier plot showing survival of an outbred (75% C57BL/6J) cohort of ΔNIC mice (n=10) and their wild-type littermate (n=1). Mecp2-null data (n=24)¹⁶ is used as a comparator.

FIG. 11 shows that ΔNIC mice have decreased body weight.

Growth curve of the backcrossed scoring cohort (see FIG. 9D-E). Graph shows mean±S.E.M. Genotypes were compared using repeated measures ΔNOVA: ****p<0.0001. Mecp2-null data (n=20)¹⁶ is used as a comparator.

FIG. 12 shows that no activity phenotype was detected for ΔNIC mice.

Behavioural analysis of ΔNIC (n=10) compared to their wildtype littermates (n=10) at 20 weeks of age (see FIG. 9F-H). Total distance travelled the Open Field test was measured during a 20 minute trial. Graphs show individual values and medians, and genotypes were compared using a t-test p=0.333.

FIG. 13 shows that ΔNIC mice have a less severe phenotype than the mildest mouse model of RTT, R133C.
A), B), C) Copy of phenotypic analysis of ΔNIC mice and wild-type littermates presented in FIG. 9D-E and FIG. S11 using EGFP-tagged R133C mice (n=10)¹⁶ as a comparator.

FIG. 14 shows that ‘STOP’ mice with transcriptionally silenced ΔNIC resemble Mecp2 nulls.
A) Western blot analysis of crude whole brain extract showing protein sizes and levels in STOP mice (n=3) compared to WT-EGFP (n=3) and ΔNIC controls (n=3), detected using a GFP antibody. Histone H3 was used as a loading control. *denotes a non-specific band detected by the GFP antibody.
B) Flow cytometry analysis of protein levels in nuclei prepared from whole brain (‘All’) and the high-NeuN subpopulation (‘Neurons’) in WT-EGFP (n=3), ΔNIC (n=3) and STOP (n=3) mice, detected using EGFP fluorescence. Graph shows mean±S.E.M and genotypes were compared using t-tests: **** denotes a p value<0.0001.
C) Phenotypic scoring of STOP mice (n=22) compared to Mecp2-null data (n=12)¹⁶. Graph shows mean scores±S.E.M.
D) Kaplan-Meier plot showing survival of STOP mice (n=14) compared to Mecp2-null data (n=24)¹⁶.

FIG. 15 shows that reactivation of ΔNIC successfully reverses neurological symptoms in MeCP2-deficient mice.
A) Timeline of the reversal experiment (results shown in B-C and FIG. 16).
B) Phenotypic scoring of Tamoxifen-injected mice from 4-28 weeks: WT^{T} (n=4), WT CreER^{T}(n=4), STOP^{T} (n=9) and STOP CreER^{T} (n=9). Graph shows mean scores±S.E.M.
C) Kaplan-Meier plot showing survival of the same cohort. Arrows indicate the timing of Tamoxifen injections. ‘^{T}’ denotes Tamoxifen-injected animals.
D) Timeline of the AAV-mediated rescue experiment (results shown in E-F and FIG. 17).
E) Phenotypic scoring of AAV9-injected mice from 5-20 weeks: WT+vehicle (n=19), Null+vehicle (n=21) and Null+ΔNIC (n=11). Graph shows mean scores±S.E.M.
F) Kaplan-Meier plot showing survival of the same animals. An arrow indicates the timing of the viral injection.

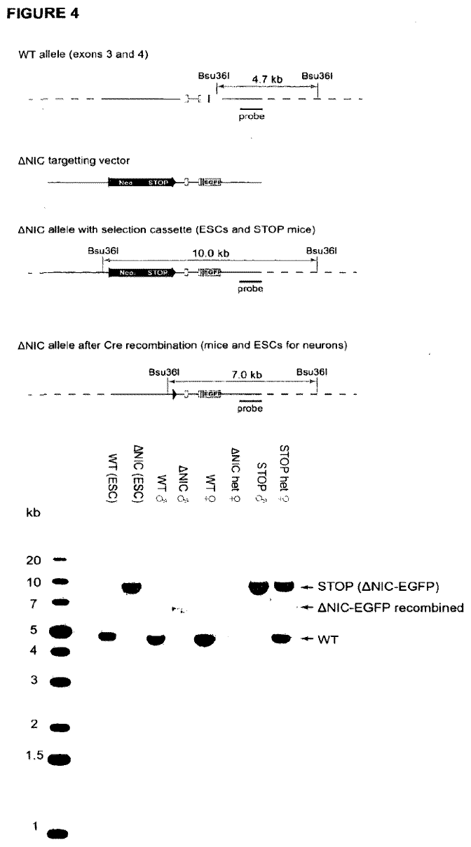

FIG. 16 shows successful reactivation of ΔNIC in Tamoxifen-injected STOP CreER mice.
A) Southern blot analysis of genomic DNA to determine the level of recombination by CreER in Tamoxifen (‘+Tmx’)-injected STOP CreER animals (n=8). One Tamoxifen-injected STOP animal was included as a negative control showing recombination was dependent on CreER. Other samples were included for reference (see restriction map in FIG. 4).
B) Protein levels in Tamoxifen-injected STOP CreER animals was determined using western blotting (upper, n=5) and flow cytometry (lower, n=3). Constitutively expressing ΔNIC mice (n=3) were used as a comparator. Graphs show mean values±S.E.M (quantification by western blotting is shown normalised to ΔNIC). Genotypes were compared using t-tests: western blotting p=0.434; flow cytometry All nuclei p=0.128 and Neuronal nuclei *p=0.016.

FIG. 17 shows that introduction of ΔNIC into wild-type mice does not have adverse consequences.
A) Phenotypic scoring of AAV9-injected mice from 5-20 weeks: WT+vehicle (n=19) Null+vehicle (n=21) and WT+ΔNIC (n=9). Graph shows mean scores±S.E.M. An arrow indicates the timing of the viral injection.
B) Design of construct used in the vector delivery of ΔNIC. Putative regulatory elements (RE) in the extended mMeP426 promoter and endogenous distal 3′-UTR are indicated. The extent of the short 229 bp region of the murine Mecp2 endogenous core promoter that is disclosed in the art^{29,38} (mMeP229) is shown relative to the mMeP426 promoter used in this construct. The RDH1 pA 3′-UTR consists of several exogenous microRNA (miR) binding sites incorporated as a ‘binding panel’ adjacent to a portion of the distal endogenous MECP2 polyadenylation signal and its accompanying regulatory elements. References with an asterisk indicate human in vitro studies, not rodent.
C) Full, annotated, sequence of the expression cassette illustrated in FIG. 17B, with flanking AAV2 ITRs. This sequence is also provided as SEQ ID NO: 65.

FIG. 18 shows an alignment of the cDNA sequence of wild type human MECP2 e1 isoform with cDNA sequences encoding polypeptide sequences in accordance with the invention and the experimental results provided herein. “Human WT” (SEQ ID NO: 18) is a cDNA sequence for the wild type MeCP2 isoform 1. “dNIC-Myc” (SEQ ID NO: 62) is a cDNA sequence for a synthetic polypeptide in accordance with the invention having deletions in the N and C-terminal sequences of MeCP2 and in the sequence linking the MBD and NID, and having a Myc tag at the C-terminus. “dNC-Myc” (SEQ ID NO: 63) is a cDNA sequence for a synthetic polypeptide in accordance with the invention having deletions in the N and C-terminal sequences of MeCP2 and having a Myc tag at the C-terminus. The sections of the cDNA sequences corresponding to the extreme N terminus of the polypeptide, providing the e1-specific sequences, the MBD, the NID, the Myc tag, a SV40 NLS, and linkers for attaching the tag and NLS, are all indicated.

FIG. 19 shows an alignment of the amino acid sequence of wild type human MeCP2 e1 isoform with polypeptide sequences in accordance with the invention and the experimental results provided herein. “Human WT” (SEQ ID NO: 3) is the amino acid sequence for the wild type MeCP2 isoform 1. “dNIC-Myc” (SEQ ID NO: 61) is the amino acid sequence for a synthetic polypeptide in accordance with the invention having deletions in the N and C-terminal sequences of MeCP2 and in the sequence linking the MBD and NID, and having a Myc tag at the C-terminus. “dNC-Myc” (SEQ ID NO: 60) is the amino acid sequence for a synthetic polypeptide in accordance with the invention having deletions in the N and C-terminal sequences of MeCP2 and having a Myc tag at the C-terminus. The sections of the amino acid sequences corresponding to the extreme N terminus of the polypeptide, having the e1-specific sequences, the MBD, the NID, the Myc tag, a SV40 NLS, and linkers for attaching the tag and NLS, are all indicated.

FIG. 20 shows an alignment of the cDNA sequence of the wild type mouse MECP2 e1 isoform, with an EGFP tag, with cDNA sequences encoding polypeptide sequences in accordance with the invention and the experimental results provided herein. “dNIC-EGFP” (SEQ ID NO: 51) is a cDNA sequence for a synthetic polypeptide in accordance with the invention having deletions in the N and C-terminal sequences of MeCP2 and in the sequence linking the MBD and NID, and having an EGFP tag at the C-terminus. “dNC-EGFP” (SEQ ID NO: 50) is a cDNA sequence for a synthetic polypeptide in accordance with the invention having deletions in the N and C-terminal sequences of MeCP2 and having an EGFP tag at the C-terminus. “WT-EGFP” (SEQ ID NO: 48) is a cDNA sequence for the wild type MeCP2 isoform 1 with an EGFP tag. “dN-EGFP” (SEQ ID NO: 49) is a cDNA sequence for a synthetic polypeptide in accordance with the invention having deletions in the N terminal sequences of MeCP2 and having an EGFP tag at the C-terminus. The sections of the cDNA sequences corresponding to the extreme N terminus of the polypeptide, providing the e1-specific sequences, the MBD, the NID, the EGFP tag, a SV40 NLS, and linkers for attaching the tag and NLS, are all indicated.

FIG. 21 shows an alignment of the amino acid sequence of wild type human MECP2 e1 isoform, with an EGFP tag, with polypeptide sequences in accordance with the invention and the experimental results provided herein. “WT-EGFP/1-748” (SEQ ID NO: 40) is an amino acid sequence for the wild type MeCP2 isoform 1 with an EGFP tag at the C-terminus. “ΔN-EGFP/1-689” (SEQ ID NO: 41) is the amino acid sequence for a synthetic polypeptide in accordance with the invention having deletions in the N terminal sequences of MeCP2 and having an EGFP tag at the C-terminus. “ΔNIC-EGFP/1-432” (SEQ ID NO: 43) is the amino acid sequence for a synthetic polypeptide in accordance with the invention having deletions in the N and C-terminal sequences of MeCP2 and in the sequence linking the MBD and NID, and having an EGFP tag at the C-terminus. “ΔNC-EGFP/1-516” (SEQ ID NO: 42) is the amino acid sequence for a synthetic polypeptide in accordance with the invention having deletions in the N and C-terminal sequences of MeCP2 and having an EGFP tag at the C-terminus. The sections of the amino acid sequences corresponding to the extreme N terminus of the polypeptide, having the e1-specific sequences, the MBD, the NID, the EGFP tag, a SV40 NLS, and linkers for attaching the tag and NLS, are all indicated.

SEQUENCES

Below are polynucleotide and amino acid sequences used in accordance with the invention.
[SEQ ID NO: 1] MeCP2 Methyl-CpG Binding Domain (MBD) polypeptide sequence
PAVPEASASPKQRRSIIRDRGPMYDDPTLPEGVVTRKLKQRKSGRSAGKYDVYLINPQGKAF
RSKVELIAYFEKVGDTSLDPNDFDFTVTGRGSPSRREQKPP
[SEQ ID NO: 2] MeCP2 NCoR/SMRT Interaction Domain (NID) polypeptide sequence
PGSVVAAAAAEAKKKAVKESSIRSVQETVLPIKKRKTRETV
[SEQ ID NO: 3] Full length human wild type MeCP2 polypeptide sequence (e1 isoform)
MAAAAAAAPSGGGGGGEEERLEEKSEDQDLQGLKDKPLKFKKVKKDKKEEKEGKHEPVQ
PSAHHSAEPAEAGKAETSEGSGSAPAVPEASASPKQRRSIIRDRGPMYDDPTLPEGVVTRK
LKQRKSGRSAGKYDVYLINPQGKAFRSKVELIAYFEKVGDTSLDPNDFDFTVTGRGSPSRR
EQKPPKKPKSPKAPGTGRGRGRPKGSGTTRPKAATSEGVQVKRVLEKSPGKLLVKMPFQT
SPGGKAEGGGATTSTQVMVIKRPGRKRKAEADPQAIPKKRGRKPGSVVAAAAAEAKKKAV
KESSIRSVQETVLPIKKRKTRETVSIEVKEVVKPLLVSTLGEKSGKGLKTCKSPGRKSKESSP
KGRSSSASSPPKKEHHHHHHHSESPKAPVPLLPPLPPPPPEPESSEDPTSPPEPQDLSSSV
CKEEKMPRGGSLESDGCPKEPAKTQPAVATAATAAEKYKHRGEGERKDIVSSSMPRPNRE
EPVDSRTPVTERVS
[SEQ ID NO: 4] Full length human wild type MeCP2 polypeptide sequence (e2 isoform)
MVAGMLGLREEKSEDQDLQGLKDKPLKFKKVKKDKKEEKEGKHEPVQPSAHHSAEPAEA
GKAETSEGSGSAPAVPEASASPKQRRSIIRDRGPMYDDPTLPEGVVTRKLKQRKSGRSAGK
YDVYLINPQGKAFRSKVELIAYFEKVGDTSLDPNDFDFTVTGRGSPSRREQKPPKKPKSPK
APGTGRGRGRPKGSGTTRPKAATSEGVQVKRVLEKSPGKLLVKMPFQTSPGGKAEGGGA
TTSTQVMVIKRPGRKRKAEADPQAIPKKRGRKPGSVVAAAAAEAKKKAVKESSIRSVQETV
LPIKKRKTRETVSIEVKEVVKPLLVSTLGEKSGKGLKTCKSPGRKSKESSPKGRSSSASSPP
KKEHHHHHHHSESPKAPVPLLPPLPPPPPEPESSEDPTSPPEPQDLSSSVCKEEKMPRGG
SLESDGCPKEPAKTQPAVATAATAAEKYKHRGEGERKDIVSSSMPRPNREEPVDSRTPVT
ERVS
[SEQ ID NO: 5] MeCP2 polypeptide sequence (e1 isoform) N-terminal to the MBD
MAAAAAAAPSGGGGGGEEERLEEKSEDQDLQGLKDKPLKFKKVKKDKKEEKEGKHEPVQ
PSAHHSAEPAEAGKAETSEGSGSA
[SEQ ID NO: 6] MeCP2 polypeptide sequence (e2 isoform) N-terminal to the MBD
MVAGMLGLREEKSEDQDLQGLKDKPLKFKKVKKDKKEEKEGKHEPVQPSAHHSAEPAEA
GKAETSEGSGSA
[SEQ ID NO: 7] MeCP2 polypeptide sequence intervening between the MBD and NID
KKPKSPKAPGTGRGRGRPKGSGTTRPKAATSEGVQVKRVLEKSPGKLLVKMPFQTSPGG
KAEGGGATTSTQVMVIKRPGRKRKAEADPQAIPKKRGRK
[SEQ ID NO: 8] MeCP2 polypeptide sequence C-terminal to the NID
SIEVKEVVKPLLVSTLGEKSGKGLKTCKSPGRKSKESSPKGRSSSASSPPKKEHHHHHHHS
ESPKAPVPLLPPLPPPPPEPESSEDPTSPPEPQDLSSSVCKEEKMPRGGSLESDGCPKEPA
KTQPAVATAATAAEKYKHRGEGERKDIVSSSMPRPNREEPVDSRTPVTERVS
[SEQ ID NO: 9] Mouse e1 specific extreme N-terminus polypeptide sequence
MAAAAATAAAAAAPSGGGGGGEEERLEEK
[SEQ ID NO: 10] Mouse e2 specific extreme N-terminus polypeptide sequence
MVAGMLGLREEK
[SEQ ID NO: 11] Human e1 specific extreme N-terminus polypeptide sequence
MAAAAAAAPSGGGGGGEEERLEEK
[SEQ ID NO: 12] Human e2 specific extreme N-terminus polypeptide sequence
MVAGMLGLREEK
[SEQ ID NO: 13] ΔNC: A truncated synthetic polypeptide sequence (from human
MeCP2)
PAVPEASASPKQRRSIIRDRGPMYDDPTLPEGVVTRKLKQRKSGRSAGKYDVYLINPQGKAF
RSKVELIAYFEKVGDTSLDPNDFDFTVTGRGSPSRREQKPPKKPKSPKAPGTGRGRGRPK
GSGTTRPKAATSEGVQVKRVLEKSPGKLLVKMPFQTSPGGKAEGGGATTSTQVMVIKRPG
RKRKAEADPQAIPKKRGRKPGSVVAAAAAEAKKKAVKESSIRSVQETVLPIKKRKTRETV
[SEQ ID NO: 14] ΔNIC: A truncated synthetic polypeptide sequence (from human
MeCP2)
PAVPEASASPKQRRSIIRDRGPMYDDPTLPEGVVTRKLKQRKSGRSAGKYDVYLINPQGKAF
RSKVELIAYFEKVGDTSLDPNDFDFTVTGRGSPSRREQKPPGSSGSSGPKKKRKVPGSVV
AAAAAEAKKKAVKESSIRSVQETVLPIKKRKTRETV
[SEQ ID NO: 15] ΔN mouse: A truncated synthetic polypeptide sequence (from mouse
MeCP2)
PAVPEASASPKQRRSIIRDRGPMYDDPTLPEGVVTRKLKQRKSGRSAGKYDVYLINPQGKAF
RSKVELIAYFEKVGDTSLDPNDFDFTVTGRGSPSRREQKPPKKPKSPKAPGTGRGRGRPK
GSGTGRPKAAASEGVQVKRVLEKSPGKLVVKMPFQASPGGKGEGGGATTSAQVMVIKRP
GRKRKAEADPQAIPKKRGRKPGSVVAAAAAEAKKKAVKESSIRSVHETVLPIKKRKTRETVS
IEVKEVVKPLLVSTLGEKSGKGLKTCKSPGRKSKESSPKGRSSSASSPPKKEHHHHHHHSE
STKAPMPLLPSPPPPEPESSEDPISPPEPQDLSSSICKEEKMPRGGSLESDGCPKEPAKTQ
PMVATTTTVAEKYKHRGEGERKDIVSSSMPRPNREEPVDSRTPVTERVS
[SEQ ID NO: 16] ΔNC mouse: A truncated synthetic polypeptide sequence (from mouse
MeCP2)
PAVPEASASPKQRRSIIRDRGPMYDDPTLPEGVVTRKLKQRKSGRSAGKYDVYLINPQGKAF
RSKVELIAYFEKVGDTSLDPNDFDFTVTGRGSPSRREQKPPKKPKSPKAPGTGRGRGRPK
GSGTGRPKAAASEGVQVKRVLEKSPGKLVVKMPFQASPGGKGEGGGATTSAQVMVIKRP
GRKRKAEADPQAIPKKRGRKPGSVVAAAAAEAKKKAVKESSIRSVHETVLPIKKRKTRETV
[SEQ ID NO: 17] ΔNIC mouse: A truncated synthetic polypeptide sequence (from mouse
MeCP2)
PAVPEASASPKQRRSIIRDRGPMYDDPTLPEGVVTRKLKQRKSGRSAGKYDVYLINPQGKAF
RSKVELIAYFEKVGDTSLDPNDFDFTVTGRGSPSRREQKPPGSSGSSGPKKKRKVPGSVV
AAAAAEAKKKAVKESSIRSVHETVLPIKKRKTRETV
[SEQ ID NO: 18] Full length human wild type MeCP2 cDNA sequence (e1 isoform)
ATGGCCGCCGCCGCCGCCGCCGCGCCGAGCGGAGGAGGAGGAGGAGGCGAGGAGG
AGAGACTGGAAGAAAAGTCAGAAGACCAGGACCTCCAGGGCCTCAAGGACAAACCCCT
CAAGTTTAAAAAGGTGAAGAAAGATAAGAAAGAAGAGAAAGAGGGCAAGCATGAGCCC
GTGCAGCCATCAGCCCACCACTCTGCTGAGCCCGCAGAGGCAGGCAAAGCAGAGACA
TCAGAAGGGTCAGGCTCCGCCCCGGCTGTGCCGGAAGCTTCTGCCTCCCCCAAACAG
CGGCGCTCCATCATCCGTGACCGGGGACCCATGTATGATGACCCCACCCTGCCTGAAG
GCTGGACACGGAAGCTTAAGCAAAGGAAATCTGGCCGCTCTGCTGGGAAGTATGATGT
GTATTTGATCAATCCCCAGGGAAAAGCCTTTCGCTCTAAAGTGGAGTTGATTGCGTACT
TCGAAAAGGTAGGCGACACATCCCTGGACCCTAATGATTTTGACTTCACGGTAACTGGG
AGAGGGAGCCCCTCCCGGCGAGAGCAGAAACCACCTAAGAAGCCCAAATCTCCCAAA
GCTCCAGGAACTGGCAGAGGCCGGGGACGCCCCAAAGGGAGCGGCACCACGAGACC
CAAGGCGGCCACGTCAGAGGGTGTGCAGGTGAAAAGGGTCCTGGAGAAAAGTCCTGG
GAAGCTCCTTGTCAAGATGCCTTTTCAAACTTCGCCAGGGGGCAAGGCTGAGGGGGGT
GGGGCCACCACATCCACCCAGGTCATGGTGATCAAACGCCCCGGCAGGAAGCGAAAA
GCTGAGGCCGACCCTCAGGCCATTCCCAAGAAACGGGGCCGAAAGCCGGGGAGTGTG
GTGGCAGCCGCTGCCGCCGAGGCCAAAAAGAAAGCCGTGAAGGAGTCTTCTATCCGA
TCTGTGCAGGAGACCGTACTCCCCATCAAGAAGCGCAAGACCCGGGAGACGGTCAGC
ATCGAGGTCAAGGAAGTGGTGAAGCCCCTGCTGGTGTCCACCCTCGGTGAGAAGAGC
GGGAAAGGACTGAAGACCTGTAAGAGCCCTGGGCGGAAAAGCAAGGAGAGCAGCCCC
AAGGGGCGCAGCAGCAGCGCCTCCTCACCCCCCAAGAAGGAGCACCACCACCATCAC
CACCACTCAGAGTCCCCAAAGGCCCCCGTGCCACTGCTCCCACCCCTGCCCCCACCTC
CACCTGAGCCCGAGAGCTCCGAGGACCCCACCAGCCCCCCTGAGCCCCAGGACTTGA
GCAGCAGCGTCTGCAAAGAGGAGAAGATGCCCAGAGGAGGCTCACTGGAGAGCGACG
GCTGCCCCAAGGAGCCAGCTAAGACTCAGCCCGCGGTTGCCACCGCCGCCACGGCCG
CAGAAAAGTACAAACACCGAGGGGAGGGAGAGCGCAAAGACATTGTTTCATCCTCCAT
GCCAAGGCCAAACAGAGAGGAGCCTGTGGACAGCCGGACGCCCGTGACCGAGAGAGT
TAGC
[SEQ ID NO: 19] ΔNC: cDNA sequence of a truncated synthetic polypeptide sequence
(from human MeCP2)
CCGGCTGTGCCGGAAGCTTCTGCCTCCCCCAAACAGCGGCGCTCCATCATCCGTGAC
CGGGGACCCATGTATGATGACCCCACCCTGCCTGAAGGCTGGACACGGAAGCTTAAG
CAAAGGAAATCTGGCCGCTCTGCTGGGAAGTATGATGTGTATTTGATCAATCCCCAGG
GAAAAGCCTTTCGCTCTAAAGTGGAGTTGATTGCGTACTTCGAAAAGGTAGGCGACACA
TCCCTGGACCCTAATGATTTTGACTTCACGGTAACTGGGAGAGGGAGCCCCTCCCGGC
GAGAGCAGAAACCACCTAAGAAGCCCAAATCTCCCAAAGCTCCAGGAACTGGCAGAGG
CCGGGGACGCCCCAAAGGGAGCGGCACCACGAGACCCAAGGCGGCCACGTCAGAGG
GTGTGCAGGTGAAAAGGGTCCTGGAGAAAAGTCCTGGGAAGCTCCTTGTCAAGATGCC
TTTTCAAACTTCGCCAGGGGGCAAGGCTGAGGGGGGTGGGGCCACCACATCCACCCA
GGTCATGGTGATCAAACGCCCCGGCAGGAAGCGAAAAGCTGAGGCCGACCCTCAGGC
CATTCCCAAGAAACGGGGCCGAAAGCCGGGGAGTGTGGTGGCAGCCGCTGCCGCCG
AGGCCAAAAAGAAAGCCGTGAAGGAGTCTTCTATCCGATCTGTGCAGGAGACCGTACT
CCCCATCAAGAAGCGCAAGACCCGGGAGACGGTC
[SEQ ID NO: 20] ΔNIC: cDNA sequence of a truncated synthetic polypeptide sequence
(from human MeCP2)
CCGGCTGTGCCGGAAGCTTCTGCCTCCCCCAAACAGCGGCGCTCCATCATCCGTGAC
CGGGGACCCATGTATGATGACCCCACCCTGCCTGAAGGCTGGACACGGAAGCTTAAG
CAAAGGAAATCTGGCCGCTCTGCTGGGAAGTATGATGTGTATTTGATCAATCCCCAGG
GAAAAGCCTTTCGCTCTAAAGTGGAGTTGATTGCGTACTTCGAAAAGGTAGGCGACACA
TCCCTGGACCCTAATGATTTTGACTTCACGGTAACTGGGAGAGGGAGCCCCTCCCGGC
GAGAGCAGAAACCACCTGGATCCAGTGGCAGCTCTGGGCCCAAGAAAAAGCGGAAGG
TGCCGGGGAGTGTGGTGGCAGCCGCTGCCGCCGAGGCCAAAAAGAAAGCCGTGAAG
GAGTCTTCTATCCGATCTGTGCAGGAGACCGTACTCCCCATCAAGAAGCGCAAGACCC
GGGAGACGGTC
[SEQ ID NO: 21] Full length mouse wild type MeCP2 cDNA sequence (el isoform)
ATGGCCGCCGCTGCCGCCACCGCCGCCGCCGCCGCCGCGCCGAGCGGAGGAGGAG
GAGGAGGCGAGGAGGAGAGACTGGAGGAAAAGTCAGAAGACCAGGATCTCCAGGGCC
TCAGAGACAAGCCACTGAAGTTTAAGAAGGCGAAGAAAGACAAGAAGGAGGACAAAGA
AGGCAAGCATGAGCCACTACAACCTTCAGCCCACCATTCTGCAGAGCCAGCAGAGGCA
GGCAAAGCAGAAACATCAGAAAGCTCAGGCTCTGCCCCAGCAGTGCCAGAAGCCTCG
GCTTCCCCCAAACAGCGGCGCTCCATTATCCGTGACCGGGGACCTATGTATGATGACC
CCACCTTGCCTGAAGGTTGGACACGAAAGCTTAAACAAAGGAAGTCTGGCCGATCTGC
TGGAAAGTATGATGTATATTTGATCAATCCCCAGGGAAAAGCTTTTCGCTCTAAAGTAGA
ATTGATTGCATACTTTGAAAAGGTGGGAGACACCTCCTTGGACCCTAATGATTTTGACTT
CACGGTAACTGGGAGAGGGAGCCCCTCCAGGAGAGAGCAGAAACCACCTAAGAAGCC
CAAATCTCCCAAAGCTCCAGGAACTGGCAGGGGTCGGGGACGCCCCAAAGGGAGCGG
CACTGGGAGACCAAAGGCAGCAGCATCAGAAGGTGTTCAGGTGAAAAGGGTCCTGGA
GAAGAGCCCTGGGAAACTTGTTGTCAAGATGCCTTTCCAAGCATCGCCTGGGGGTAAG
GGTGAGGGAGGTGGGGCTACCACATCTGCCCAGGTCATGGTGATCAAACGCCCTGGC
AGAAAGCGAAAAGCTGAAGCTGACCCCCAGGCCATTCCTAAGAAACGGGGTAGAAAGC
CTGGGAGTGTGGTGGCAGCTGCTGCAGCTGAGGCCAAAAAGAAAGCCGTGAAGGAGT
CTTCCATACGGTCTGTGCATGAGACTGTGCTCCCCATCAAGAAGCGCAAGACCCGGGA
GACGGTCAGCATCGAGGTCAAGGAAGTGGTGAAGCCCCTGCTGGTGTCCACCCTTGG
TGAGAAAAGCGGGAAGGGACTGAAGACCTGCAAGAGCCCTGGGCGTAAAAGCAAGGA
GAGCAGCCCCAAGGGGCGCAGCAGCAGTGCCTCCTCCCCACCTAAGAAGGAGCACCA
TCATCACCACCATCACTCAGAGTCCACAAAGGCCCCCATGCCACTGCTCCCATCCCCA
CCCCCACCTGAGCCTGAGAGCTCTGAGGACCCCATCAGCCCCCCTGAGCCTCAGGAC
TTGAGCAGCAGCATCTGCAAAGAAGAGAAGATGCCCCGAGGAGGCTCACTGGAAAGC
GATGGCTGCCCCAAGGAGCCAGCTAAGACTCAGCCTATGGTCGCCACCACTACCACAG
TTGCAGAAAAGTACAAACACCGAGGGGAGGGAGAGCGCAAAGACATTGTTTCATCTTC
CATGCCAAGGCCAAACAGAGAGGAGCCTGTGGACAGCCGGACGCCCGTGACCGAGAG
AGTTAGCTCT
[SEQ ID NO: 22] ΔN mouse: cDNA for a truncated synthetic polypeptide sequence (from
mouse MeCP2)
CCAGCAGTGCCAGAAGCCTCGGCTTCCCCCAAACAGCGGCGCTCCATTATCCGTGACC
GGGGACCTATGTATGATGACCCCACCTTGCCTGAAGGTTGGACACGAAAGCTTAAACA
AAGGAAGTCTGGCCGATCTGCTGGAAAGTATGATGTATATTTGATCAATCCCCAGGGAA
AAGCTTTTCGCTCTAAAGTAGAATTGATTGCATACTTTGAAAAGGTGGGAGACACCTCC
TTGGACCCTAATGATTTTGACTTCACGGTAACTGGGAGAGGGAGCCCCTCCAGGAGAG
AGCAGAAACCACCTAAGAAGCCCAAATCTCCCAAAGCTCCAGGAACTGGCAGGGGTCG
GGGACGCCCCAAAGGGAGCGGCACTGGGAGACCAAAGGCAGCAGCATCAGAAGGTGT
TCAGGTGAAAAGGGTCCTGGAGAAGAGCCCTGGGAAACTTGTTGTCAAGATGCCTTTC
CAAGCATCGCCTGGGGGTAAGGGTGAGGGAGGTGGGGCTACCACATCTGCCCAGGTC
ATGGTGATCAAACGCCCTGGCAGAAAGCGAAAAGCTGAAGCTGACCCCCAGGCCATTC
CTAAGAAACGGGGTAGAAAGCCTGGGAGTGTGGTGGCAGCTGCTGCAGCTGAGGCCA
AAAAGAAAGCCGTGAAGGAGTCTTCCATACGGTCTGTGCATGAGACTGTGCTCCCCAT
CAAGAAGCGCAAGACCCGGGAGACGGTCAGCATCGAGGTCAAGGAAGTGGTGAAGCC
CCTGCTGGTGTCCACCCTTGGTGAGAAAAGCGGGAAGGGACTGAAGACCTGCAAGAG
CCCTGGGCGTAAAAGCAAGGAGAGCAGCCCCAAGGGGCGCAGCAGCAGTGCCTCCTC
CCCACCTAAGAAGGAGCACCATCATCACCACCATCACTCAGAGTCCACAAAGGCCCCC
ATGCCACTGCTCCCATCCCCACCCCCACCTGAGCCTGAGAGCTCTGAGGACCCCATCA
GCCCCCCTGAGCCTCAGGACTTGAGCAGCAGCATCTGCAAAGAAGAGAAGATGCCCC
GAGGAGGCTCACTGGAAAGCGATGGCTGCCCCAAGGAGCCAGCTAAGACTCAGCCTA
TGGTCGCCACCACTACCACAGTTGCAGAAAAGTACAAACACCGAGGGGAGGGAGAGC
GCAAAGACATTGTTTCATCTTCCATGCCAAGGCCAAACAGAGAGGAGCCTGTGGACAG
CCGGACGCCCGTGACCGAGAGAGTTAGCTGT
[SEQ ID NO: 23] ΔNC mouse: cDNA for a truncated synthetic polypeptide sequence
(from mouse MeCP2)
CCAGCAGTGCCAGAAGCCTCGGCTTCCCCCAAACAGCGGCGCTCCATTATCCGTGACC
GGGGACCTATGTATGATGACCCCACCTTGCCTGAAGGTTGGACACGAAAGCTTAAACA
AAGGAAGTCTGGCCGATCTGCTGGAAAGTATGATGTATATTTGATCAATCCCCAGGGAA
AAGCTTTTCGCTCTAAAGTAGAATTGATTGCATACTTTGAAAAGGTGGGAGACACCTCC
TTGGACCCTAATGATTTTGACTTCACGGTAACTGGGAGAGGGAGCCCCTCCAGGAGAG
AGCAGAAACCACCTAAGAAGCCCAAATCTCCCAAAGCTCCAGGAACTGGCAGGGGTCG
GGGACGCCCCAAAGGGAGCGGCACTGGGAGACCAAAGGCAGCAGCATCAGAAGGTGT
TCAGGTGAAAAGGGTCCTGGAGAAGAGCCCTGGGAAACTTGTTGTCAAGATGCCTTTC
CAAGCATCGCCTGGGGGTAAGGGTGAGGGAGGTGGGGCTACCACATCTGCCCAGGTC
ATGGTGATCAAACGCCCTGGCAGAAAGCGAAAAGCTGAAGCTGACCCCCAGGCCATTC
CTAAGAAACGGGGTAGAAAGCCTGGGAGTGTGGTGGCAGCTGCTGCAGCTGAGGCCA
AAAAGAAAGCCGTGAAGGAGTCTTCCATACGGTCTGTGCATGAGACTGTGCTCCCCAT
CAAGAAGCGCAAGACCCGGGAGACGGTC
[SEQ ID NO: 24] ΔNIC mouse: cDNA for a truncated synthetic polypeptide sequence
(from mouse MeCP2)
CCAGCAGTGCCAGAAGCCTCGGCTTCCCCCAAACAGCGGCGCTCCATTATCCGTGACC
GGGGACCTATGTATGATGACCCCACCTTGCCTGAAGGTTGGACACGAAAGCTTAAACA
AAGGAAGTCTGGCCGATCTGCTGGAAAGTATGATGTATATTTGATCAATCCCCAGGGAA
AAGCTTTTCGCTCTAAAGTAGAATTGATTGCATACTTTGAAAAGGTGGGAGACACCTCC
TTGGACCCTAATGATTTTGACTTCACGGTAACTGGGAGAGGGAGCCCCTCCAGGAGAG
AGCAGAAACCACCTGGATCCAGTGGCAGCTCTGGGCCCAAGAAAAAGCGGAAGGTGC
CTGGGAGTGTGGTGGCAGCTGCTGCAGCTGAGGCCAAAAAGAAAGCCGTGAAGGAGT
CTTCCATACGGTCTGTGCATGAGACTGTGCTCCCCATCAAGAAGCGCAAGACCCGGGA
GACGGTC
[SEQ ID NO: 25] Mouse e1 specific extreme N-terminus cDNA sequence
ATGGCCGCCGCTGCCGCCACCGCCGCCGCCGCCGCCGCGCCGAGCGGAGGAGGAG
GAGGAGGCGAGGAGGAGAGACTGGAGGAAAAG
[SEQ ID NO: 26] Mouse e2 specific extreme N-terminus cDNA sequence
ATGGTAGCTGGGATGTTAGGGCTCAGGGAGGAAAAGGGAGGAAAAG
[SEQ ID NO: 27] Human e1 specific extreme N-terminus cDNA sequence
ATGGCCGCCGCCGCCGCCGCCGCGCCGAGCGGAGGAGGAGGAGGAGGCGAGGAGG
AGAGACTGGAAGAAAAG
[SEQ ID NO: 28] Human e2 specific extreme N-terminus cDNA sequence
ATGGTAGCTGGGATGTTAGGGCTCAGGGAAGAAAAG
[SEQ ID NO: 29] Full length mouse wild type MeCP2 polypeptide sequence (e1 isoform)
MAAAAATAAAAAAPSGGGGGGEEERLEEKSEDQDLQGLRDKPLKFKKAKKDKKEDKEGK
HEPLQPSAHHSAEPAEAGKAETSESSGSAPAVPEASASPKQRRSIIRDRGPMYDDPTLPEG
VVTRKLKQRKSGRSAGKYDVYLINPQGKAFRSKVELIAYFEKVGDTSLDPNDFDFTVTGRGS
PSRREQKPPKKPKSPKAPGTGRGRGRPKGSGTGRPKAAASEGVQVKRVLEKSPGKLVVK
MPFQASPGGKGEGGGATTSAQVMVIKRPGRKRKAEADPQAIPKKRGRKPGSVVAAAAAE
AKKKAVKESSIRSVHETVLPIKKRKTRETVSIEVKEVVKPLLVSTLGEKSGKGLKTCKSPGRK
SKESSPKGRSSSASSPPKKEHHHHHHHSESTKAPMPLLPSPPPPEPESSEDPISPPEPQDL
SSSICKEEKMPRGGSLESDGCPKEPAKTQPMVATTTTVAEKYKHRGEGERKDIVSSSMPR
PNREEPVDSRTPVTERVS
[SEQ ID NO: 30] Full length mouse wild type MeCP2 polypeptide sequence (e2 isoform)
MVAGMLGLREEKSEDQDLQGLRDKPLKFKKAKKDKKEDKEGKHEPLQPSAHHSAEPAEA
GKAETSESSGSAPAVPEASASPKQRRSIIRDRGPMYDDPTLPEGVVTRKLKQRKSGRSAGK
YDVYLINPQGKAFRSKVELIAYFEKVGDTSLDPNDFDFTVTGRGSPSRREQKPPKKPKSPK
APGTGRGRGRPKGSGTGRPKAAASEGVQVKRVLEKSPGKLVVKMPFQASPGGKGEGGG
ATTSAQVMVIKRPGRKRKAEADPQAIPKKRGRKPGSVVAAAAAEAKKKAVKESSIRSVHET
VLPIKKRKTRETVSIEVKEVVKPLLVSTLGEKSGKGLKTCKSPGRKSKESSPKGRSSSASSP
PKKEHHHHHHHSESTKAPMPLLPSPPPPEPESSEDPISPPEPQDLSSSICKEEKMPRGGSL
ESDGCPKEPAKTQPMVATTTTVAEKYKHRGEGERKDIVSSSMPRPNREEPVDSRTPVTER
VS
[SEQ ID NO: 31] Full length mouse wild type MeCP2 cDNA sequence (e2 isoform)
ATGGTAGCTGGGATGTTAGGGCTCAGGGAGGAAAAGTCAGAAGACCAGGATCTCCAG
GGCCTCAGAGACAAGCCACTGAAGTTTAAGAAGGCGAAGAAAGACAAGAAGGAGGACA
AAGAAGGCAAGCATGAGCCACTACAACCTTCAGCCCACCATTCTGCAGAGCCAGCAGA
GGCAGGCAAAGCAGAAACATCAGAAAGCTCAGGCTCTGCCCCAGCAGTGCCAGAAGC
CTCGGCTTCCCCCAAACAGCGGCGCTCCATTATCCGTGACCGGGGACCTATGTATGAT
GACCCCACCTTGCCTGAAGGTTGGACACGAAAGCTTAAACAAAGGAAGTCTGGCCGAT
CTGCTGGAAAGTATGATGTATATTTGATCAATCCCCAGGGAAAAGCTTTTCGCTCTAAA
GTAGAATTGATTGCATACTTTGAAAAGGTGGGAGACACCTCCTTGGACCCTAATGATTT
TGACTTCACGGTAACTGGGAGAGGGAGCCCCTCCAGGAGAGAGCAGAAACCACCTAA
GAAGCCCAAATCTCCCAAAGCTCCAGGAACTGGCAGGGGTCGGGGACGCCCCAAAGG
GAGCGGCACTGGGAGACCAAAGGCAGCAGCATCAGAAGGTGTTCAGGTGAAAAGGGT
CCTGGAGAAGAGCCCTGGGAAACTTGTTGTCAAGATGCCTTTCCAAGCATCGCCTGGG
GGTAAGGGTGAGGGAGGTGGGGCTACCACATCTGCCCAGGTCATGGTGATCAAACGC
CCTGGCAGAAAGCGAAAAGCTGAAGCTGACCCCCAGGCCATTCCTAAGAAACGGGGTA
GAAAGCCTGGGAGTGTGGTGGCAGCTGCTGCAGCTGAGGCCAAAAAGAAAGCCGTGA
AGGAGTCTTCCATACGGTCTGTGCATGAGACTGTGCTCCCCATCAAGAAGCGCAAGAC
CCGGGAGACGGTCAGCATCGAGGTCAAGGAAGTGGTGAAGCCCCTGCTGGTGTCCAC
CCTTGGTGAGAAAAGCGGGAAGGGACTGAAGACCTGCAAGAGCCCTGGGCGTAAAAG
CAAGGAGAGCAGCCCCAAGGGGCGCAGCAGCAGTGCCTCCTCCCCACCTAAGAAGGA
GCACCATCATCACCACCATCACTCAGAGTCCACAAAGGCCCCCATGCCACTGCTCCCA
TCCCCACCCCCACCTGAGCCTGAGAGCTCTGAGGACCCCATCAGCCCCCCTGAGCCT
CAGGACTTGAGCAGCAGCATCTGCAAAGAAGAGAAGATGCCCCGAGGAGGCTCACTG
GAAAGCGATGGCTGCCCCAAGGAGCCAGCTAAGACTCAGCCTATGGTCGCCACCACTA
CCACAGTTGCAGAAAAGTACAAACACCGAGGGGAGGGAGAGCGCAAAGACATTGTTTC
ATCTTCCATGCCAAGGCCAAACAGAGAGGAGCCTGTGGACAGCCGGACGCCCGTGAC
CGAGAGAGTTAGC

### EXPERIMENTAL RESULTS

### 1. Materials and Methods

### Nomenclature

According to convention, all amino acid numbers given in the following refer to the e2 isoform. Numbers refer to homologous amino acids in human (NCBI accession P51608) and mouse (NCBI accession Q9Z2D6) until residue 385 where there is a two amino acid insertion in the human protein.

### Mutation Analysis

Mutational data was collected as described previously⁴: causative RTT-causing missense mutations were extracted from the RettBASE dataset¹³; and polymorphisms identified in healthy hemizygous males were extracted from the Exome Aggregation Consortium (ExAC) database¹⁴.

### Design of Shortened MeCP2 Proteins

The MBD and NID were defined as residues 72-173 and 272-312, respectively. All three constructs retain the extreme N-terminal sequences encoded by exons 1 and 2—present in isoforms e1 and e2 respectively. They also include the first three amino acids of exons 3 (EEK) to preserve the splice acceptor site. The intervening region (1) was replaced in ΔNIC by the NLS of SV40 preceded by a flexible linker. The sequence of the NLS is PKKKRKV (SEQ ID NO: 32) (DNA sequence: CCCAAGAAAAAGCGGAAGGTG (SEQ ID NO: 33)) and of the linker is GSSGSSG (SEQ ID NO: 34) (DNA sequence: GGATCCAGTGGCAGCTCTGGG (SEQ ID NO: 35)). All three proteins were C-terminally tagged with EGFP connected by a linker. To be consistent with a previous study tagging full-length MeCP2¹⁶, the linker sequence CKDPPVAT (SEQ ID NO: 36) (DNA sequence: TGTAAGGATCCACCGGTCGCCACC (SEQ ID NO: 37)) was used to connect the C-terminus of ΔN to EGFP. To connect the NID to the EGFP tag in ΔNC and ΔNIC, the flexible GSSGSSG (SEQ ID NO: 38) linker was used instead (DNA sequence: GGGAGCTCCGGCAGTTCTGGA (SEQ ID NO: 39)). The amino acid sequences of the e1 and e2 isoforms for WT-EGFP, ΔN-EGFP, ΔNC-EGFP and ΔNIC-EGFP polypeptides are provided herein as SEQ ID NOs 40-47, respectively. The cDNA sequences of the e1 and e2 isoforms for the WT-EGFP, ΔN-EGFP, ΔNC-EGFP and ΔNIC-EGFP polypeptides are provided herein as SEQ ID NOs 48-55, respectively.

For expression in cultured cells, cDNA sequences encoding e2 isoforms of the MeCP2 deletion series were synthesised (GeneArt, Thermo Fisher Scientific) and cloned into the pEGFPN1 vector (Clontech) using XhoI and NotI restriction sites (NEB). Point mutations (R111 G and R306C) were inserted into the WT-EGFP plasmid using the QuikChange II XL Site-Directed Mutagenesis Kit (Agilent Technologies). Primer sequences for R111 G: Forward TGGACACGAAAGCTTAAACAAGGGAAGTCTGGCC (SEQ ID NO: 56) and Reverse GGCCAGACTTCCCTTGTTTAAGCTTTCGTGTCCA (SEQ ID NO: 57); and R306C: Forward CTCCCGGGTCTTGCACTTCTTGATGGGGA (SEQ ID NO: 58) and Reverse TCCCCATCAAGAAGTGCAAGACCCGGGAG (SEQ ID NO: 59). For ES cell targeting, genomic sequences encoding exons 3 and 4 of the EGFP-tagged shortened proteins were synthesised (GeneArt, Thermo Fisher Scientific) and cloned into a previously used²⁴ targeting vector using MfeI restriction sites (NEB). This vector contains a Neomycin resistance gene followed by a transcriptional ‘STOP’ cassette flanked by LoxP sites (‘floxed’) in intron 2.

For viral delivery of shortened MeCP2 proteins, Myc epitope tagged proteins were prepared. The amino acids sequences of human ΔNC-Myc and ΔNIC-Myc polypeptides are provided herein as SEQ ID NOs 60-61, respectively. The cDNA sequences of human ΔNC-Myc and ΔNIC-Myc polypeptides are provided herein as SEQ ID NOs 62-63, respectively.

### Cell Culture

HeLa and NIH-3T3 cells were grown in DMEM (Gibco) supplemented with 10% foetal bovine serum (FBS; Gibco) and 1% Penicillin-Streptomycin (Gibco). ES cells were grown in Glasgow MEM (Gibco) supplemented with foetal bovine serum (FBS; Gibco-batch tested), 1% Non-essential amino acids (Gibco), 1% Sodium Pyruvate (Gibco), 0.1% β-mercaptoethanol (Gibco) and LIF (ESGRO).

### Immunoprecipitation

HeLa cells were transfected with pEGFPN1-MeCP2 plasmids using JetPEI (PolyPlus Transfection) and harvested after 24-48 hours. Nuclear extracts were prepared using Benzonase (Sigma E1014-25KU) and 150 mM NaCl, and MeCP2-EGFP complexes were captured using GFP-Trap_A beads (Chromotek) as described previously⁴. Proteins were analysed by western blotting using antibodies to GFP (NEB #2956), NCoR (Bethyl A301-146A), HDAC3 (Sigma 3E11) and TBL1 XR1 (Bethyl A300-408A), all at a dilution of 1:1000; followed by LI-COR secondary antibodies: IRDye® 800CW Donkey anti-Mouse (926-32212) and IRDye® 800CW Donkey anti-Rabbit (926-32213) or IRDye® 680LT Donkey anti-Rabbit (926-68023) at a dilution of 1:10,000.

### Recruitment Assay

NIH-3T3 cells were seeded on coverslips in 6 well plates (25,000 cells per well) and transfected with 2 μg plasmid DNA (pEGFPN1-MeCP2 and pmCherry-TBL1X) using JetPEI (PolyPlus Transfection). After 48 hours, cells were fixed with 4% (w/v) paraformaldehyde, stained with DAPI (Sigma) and then mounted using ProLong Diamond (Life Technologies). Fixed cells were photographed using confocal microscopy (Leica SP5).

### Generation of Knock-In Mice

Targeting vectors were introduced into 129/Ola E14 TG2a ES cells by electroporation, and G418-resistant clones with correct targeting at the Mecp2 locus were identified by PCR and Southern blot screening. CRISPR-Cas9 technology was used to increase the targeting efficiency of ΔN and ΔNIC lines: the guide RNA sequence (GGTTGTGACCCGCCATGGAT) (SEQ ID NO: 64) was cloned into pX330-U6-Chimeric_BB-CBh-hSpCas9 (a gift from Feng Zhang; Addgene plasmid #42230²⁵), which was introduced into the ES cells with the targeting vectors. This introduced a double-strand cut in intron 2 of the wild-type gene (at the site of the NeoSTOP cassette in the targeting vector). Mice were generated from ES cells as previously described²⁶. The ‘floxed’ NeoSTOP cassette was removed in vivo by crossing chimaeras with homozygous females from the transgenic CMV-Cre deleter strain (JAX Stock #006054) on a C57BL/6J background. The CMV-Cre transgene was subsequently bred out. All mice used in this study were bred and maintained at the University of Edinburgh animal facilities under standard conditions and procedures were carried out by staff licensed by the UK Home Office and according with the Animal and Scientific Procedures Act 1986.

### Biochemical Characterisation of Knock-In Mice

For biochemical analysis, brains were harvested by snap-freezing in liquid nitrogen at 6-13 weeks of age, unless otherwise stated. Brains of hemizygous male mice were used for all analysis, unless otherwise stated. For Southern blot analysis, half brains were homogenised in 50 mM Tris Cl pH7.5, 100 mM NaCl, 5 mM EDTA and treated with 0.4 mg/ml Proteinase K in 1% SDS at 55° C. overnight. Samples were treated with 0.1 mg/ml RNAseA for 1-2 hours at 37° C., before phenol:chloroform extraction of genomic DNA. Genomic DNA was purified from ES cells using Puregene Core Kit A (Qiagen) according to manufacturer's instructions for cultured cells. Genomic DNA was digested with restriction enzymes (NEB), separated by agarose gel electrophoresis and transferred onto ZetaProbe membranes (BioRad). DNA probes homologous to either exon 4 or the end of the 3′ homology arm were radioactively labelled with [α32]dCTP (Perkin Elmer) using the Prime-a-Gene Labeling System (Promega). Blots were probed overnight, washed, and exposed in Phosphorimager cassettes before scanning on a Typhoon FLA 7000. Bands were quantified using ImageQuant software.

Protein levels in whole brain crude extracts were quantified using western blotting. Extracts were prepared as described previously¹⁶, and blots were probed with antibodies to GFP (NEB #2956) or MeCP2 (Sigma M6818), both at a dilution of 1:1,000, followed by LI-COR secondary antibodies (listed above). Histone H3 (Abcam ab1791) was used as a loading control (dilution 1:10,000). Levels were quantified using Image Studio Lite Ver 4.0 software and compared using t-tests. WT-EGFP mice¹⁶ were used as controls.

For flow cytometry analysis, fresh brains were harvested from 12 week-old animals and Dounce-homogenised in 5 ml homogenisation buffer (320 mM sucrose, 5 mM CaCl2, 3 mM Mg(Ac)2, 10 mM Tris HCl pH.7.8, 0.1 mM EDTA, 0.1% NP40, 0.1 mM PMSF, 14.3 mM β-mercaptoethanol, protease inhibitors (Roche)), and 5 ml of 50% OptiPrep gradient centrifugation medium (50% Optiprep (Sigma D1556-250ML), 5 mM CaCl2, 3 mM Mg(Ac)2, 10 mM Tris HCl pH7.8, 0.1M PMSF, 14.3 mM β-mercaptoethanol) was added. This was layered on top of 10 ml of 29% OptiPrep solution (v/v in H2O) in Ultra clear Beckman Coulter centrifuge tubes, and samples were centrifuged at 7,500 rpm for 30 mins, 4° C. Pelleted nuclei were resuspended in Resuspension buffer (20% glycerol in DPBS with protease inhibitors (Roche)). For flow cytometry analysis, nuclei were pelleted at 600×g (5 mins, 4° C.), washed in 1 ml PBTB (5% (w/v) BSA, 0.1% Triton X-100 in DPBS with protease inhibitors (Roche)), and then resuspended in 250 μl PBTB. To stain for NeuN, 10 μl of NeuN-A60 antibody (Millipore MAB377) was conjugated to Alexa Fluor 647 (APEX Antibody Labelling Kit, Invitrogen A10475), added at a dilution of 1:125 and incubated under rotation for 45 mins at 4° C. Flow cytometry (BD LSRFortessa SORP) was used to obtain the mean EGFP expression for the total nuclei (n=50,000 per sample) and the high NeuN (neuronal) subpopulation (n>8,000 per sample), and genotypes were compared using t-tests. WT-EGFP mice¹⁶ were used as controls.

To determine mRNA levels, RNA was purified and reverse transcribed from half brains; and Mecp2 and Cyclophilin A transcripts were analysed by qPCR as previously described¹⁶. mRNA levels in ΔNIC mice were compared to wild-type littermates using a t-test.

### Phenotypic Characterisation of Knock-In Mice

Consistent with a previous study¹⁶, mice were backcrossed four generations to reach ˜94% C57BL/6J before undergoing phenotypic characterisation. Two separate cohorts, each consisting of 10 mutant animals and 10 wild-type littermates, were produced for each novel knock-in line. One cohort was scored and weighed regularly from 4-52 weeks of age as previously described^{24,27}. Survival was graphed using Kaplan Meier plots. (A preliminary outbred [75% C57BL/6J] cohort of 7 ΔNC mice and 9 wild-type littermates was also scored.) The second backcrossed cohort underwent behavioural analysis at 20-21 weeks of age (see ²⁷ and ¹⁶ for detailed protocols). Tests were performed over a two-week period: Elevated Plus Maze on day 1, Open Field test on day 2, and Accelerating Rotarod test on days 6-9 (one day of training followed by three days of trials). All analysis was performed blind to genotype.

### Statistical Analysis

Growth curves were compared using repeated measures ΔNOVA. For behavioural analysis, when all data fitted a normal distribution (Open Field centre time and distance travelled), genotypes were compared using t-tests. If not (Elevated Plus Maze time in arms and Accelerating Rotarod latency to fall), genotypes were compared using Kolmogorov-Smirnov tests. Change in performance over time in the Accelerating Rotarod test was determined using Friedman tests.

### Genetic Reactivation of Minimal MeCP2 (ΔNIC)

Transcriptionally silent minimal MeCP2 (ΔNIC) was reactivated in symptomatic null-like ‘STOP’ mice following the procedure used in²⁷. In short, the ΔNIC Mecp2 allele was inactivated by the retention of the NeoSTOP cassette in intron 2 by mating chimaeras with wild-type females instead of deleter mice. Resulting STOP/+females were crossed with heterozygous Cre-ER transgenic males (JAX Stock #004682) to produce males of four genotypes (87.5% C57BL/6J). A cohort consisting of all four genotypes WT (n=4), WT CreER (n=4), STOP (n=9) and STOP CreER (n=9), was scored and weighed weekly from 4 weeks of age. From 6 weeks (when STOP and STOP CreER mice displayed RTT-like symptoms), all individuals were given a series of Tamoxifen injections: two weekly followed by five daily, each at a dose of 100 μg/g body weight. Brain tissue from Tamoxifen-treated STOP CreER (n=8), WT (n=1) and WT CreER (n=1) animals was harvested at 28 weeks of age (after successful symptom reversal in STOP CreER mice) for biochemical analysis. Brain tissue from one Tamoxifen-treated STOP mouse was also included in the biochemical analysis (methods described above).

### Vector Delivery of Minimal MeCP2 (ΔNIC)

Minimal MeCP2 (ΔNIC) AAV vector was tested in Mecp2-null and WT mice maintained on a C57BL/6 background. Recombinant AAV vector particles were generated at the UNC Gene Therapy Center Vector Core facility. Self-complementary AAV (scAAV) particles (AAV2 ITR-flanked genomes packaged into AAV9 capsids) were produced from suspension HEK293 cells transfected using polyethyleneimine (Polysciences, Warrington, PA) with helper plasmids (pXX6-80, pGSK2/9) and a plasmid containing the ITR-flanked ΔNIC transgene construct. The construct used is illustrated in FIG. 17B, and the annotated sequence (SEQ ID NO: 65) of the ITR-flanked ΔNIC transgene construct is shown in FIG. 17C. For translational relevance, the ΔNIC-expressing construct utilized the equivalent human MECP2_e1 coding sequence and with a small C-terminal Myc epitope tag replacing the EGFP tag used in other experiments. The transgene was under the control of an extended endogenous Mecp2 promoter fragment (MeP426) incorporating additional promoter regulatory elements and a putative silencer element (FIG. 17B,C). The construct also incorporated a novel 3′-UTR consisting of a fragment of the endogenous MECP2 3′UTR together with a selected panel of binding sites for miRNAs known to be involved in regulation of Mecp³⁹⁻⁴¹ (FIG. 17B,C). Virus production was performed as previously described²⁸, and vector prepared in a final formulation of high-salt PBS (containing 350 mM total NaCl) supplemented with 5% sorbitol. For brain injection into mice, direct bilateral injections of virus (3 μl per site; dose=1×10¹¹ viral genome per mouse) were delivered into the neuropil of unanaesthetised P1/2 males, as described previously²⁹. Control injections were made using the same diluent lacking vector (‘vehicle control’). The injected pups were returned to the home cage and assessed weekly as described above.

### 2. Results

The amino acid sequence of MeCP2 is highly conserved throughout vertebrate species (FIG. 1A), suggesting that most of the protein is subject to purifying selection. This supports the widely-held view that its interactions with multiple binding partners are of functional importance: with which MeCP2 has been implicated in several cellular pathways required for proper neuronal function^{11,3}. An alternative picture emerges when analysing the distribution of RTT-causing missense mutations, highlighting only the MBD and NID—a small minority of the protein—as critical (FIG. 1A). Furthermore, exome sequencing data collected from healthy individuals shows a large number of polymorphisms in the other regions of the protein (FIG. 1A), suggesting these sequences are dispensable. To test whether the MBD and NID might be sufficient for MeCP2 function, we designed a stepwise series of deletions of the endogenous gene to remove regions N-terminal to the MBD (ΔN), C-terminal to the NID (ΔC) and the intervening amino acids between these domains (ΔI) (FIG. 1B). The intervening region was replaced by a nuclear localisation signal (NLS) sequence derived from SV40 virus, connected by short linkers. The Mecp2 gene has four exons, with transcripts alternatively spliced to produce two isoforms that differ only at the extreme N-termini³⁰. To maintain the Mecp2 gene structure in the knock-in mice, the constructs retained exons 1 and 2 as well as the first 10 bp of exon 3 (splice acceptor site), resulting in the inclusion of 29 and 12 N-terminal amino acids for isoforms e1 and e2, respectively (FIG. 2A-B, 3, 4). A C-terminal EGFP tag was added to facilitate detection and recovery, as tagging does not affect MeCP2 function in mice¹⁶ (FIG. 1B). Taking into account mapped binding sites, structural information and evolutionary conservation, we encompassed the MBD as residues 72-173 and the NID as residues 272-312 (FIG. S1C-D). The proportion of native MeCP2 protein sequence retained in ΔN, ΔNC and ΔNIC is 88%, 52% and 32% of wild-type, respectively.

We first tested whether the shortened MeCP2 proteins retained the ability to interact with methylated DNA and the NCoR/SMRT co-repressor complex using cell culture-based assays. All three protein derivatives immunoprecipitated endogenous NCoR/SMRT complex components when overexpressed in HeLa cells, whereas this interaction was abolished in the negative control NID mutant, R306C (FIG. 1C). To assay mCpG binding, we asked whether expressed proteins localised to mCpG-rich pericentric heterochromatic foci in mouse fibroblasts. Previous work established that localisation of wild-type MeCP2 to these foci is dependent on both DNA methylation^{31,32} and MBD functionality³³. All three shortened versions of MeCP2 localised to heterochromatic foci, whereas a negative control MBD mutant (R111 G) showed a diffuse nuclear distribution (FIG. 1D). To determine whether the shortened proteins could bind chromatin and the NCoR/SMRT complex simultaneously, we asked if they were able to recruit TBL1X, an NCoR/SMRT subunit that binds directly to MeCP2⁴, to heterochromatin. Over-expressed TBL1X-mCherry lacks an NLS and is therefore cytoplasmic, but in the presence of over-expressed MeCP2 it is efficiently recruited to heterochromatic foci⁴. All shortened MeCP2 proteins likewise recruited TBL1X to the heterochromatic foci, demonstrating their ability to bridge DNA with the co-repressor (FIG. 1E). The MeCP2 NID mutant control (R306C) itself localised correctly, but as described previously⁴ was unable to relocate TBL1X from the cytoplasm (FIG. 1E). These three assays confirm that all shortened proteins retain the ability to bind methylated DNA and the NCoR/SMRT complex and form a bridge between them.

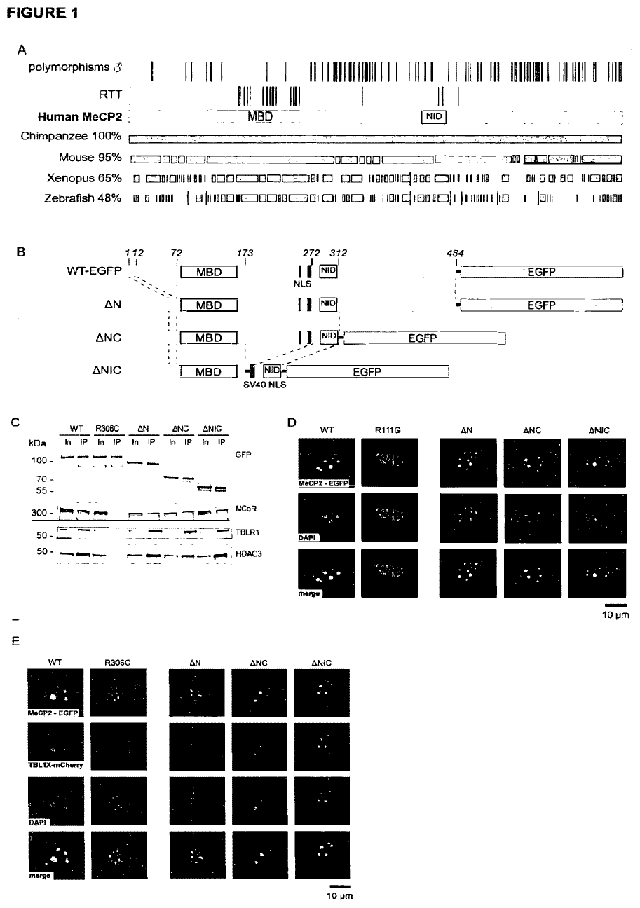

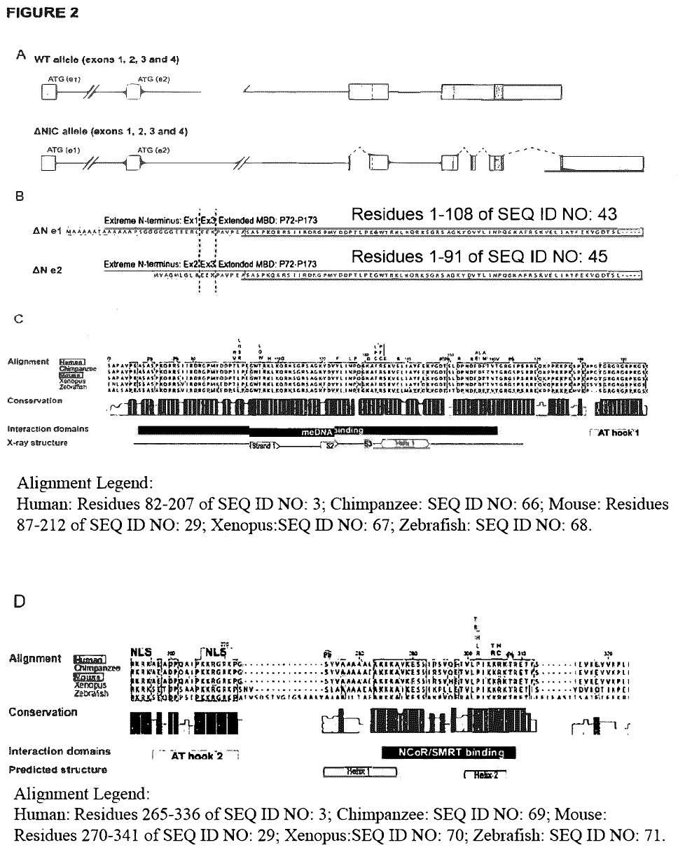

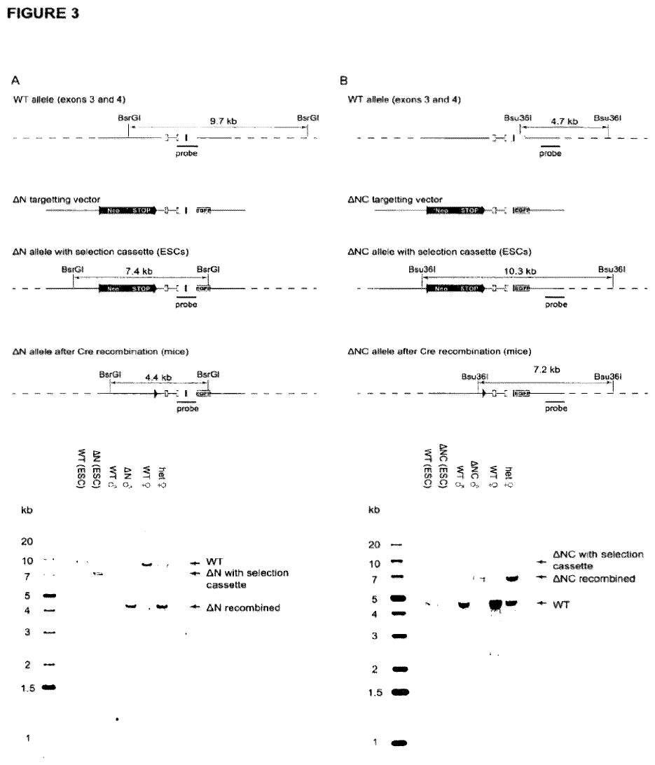

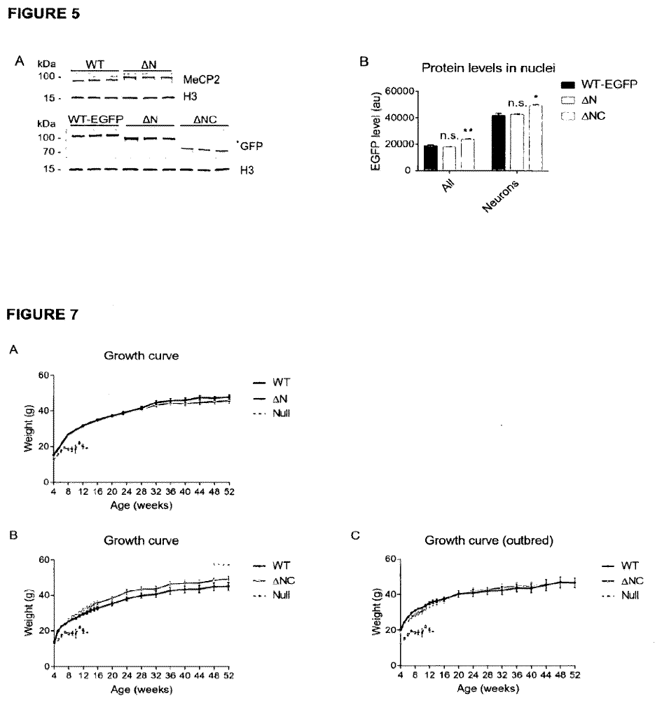

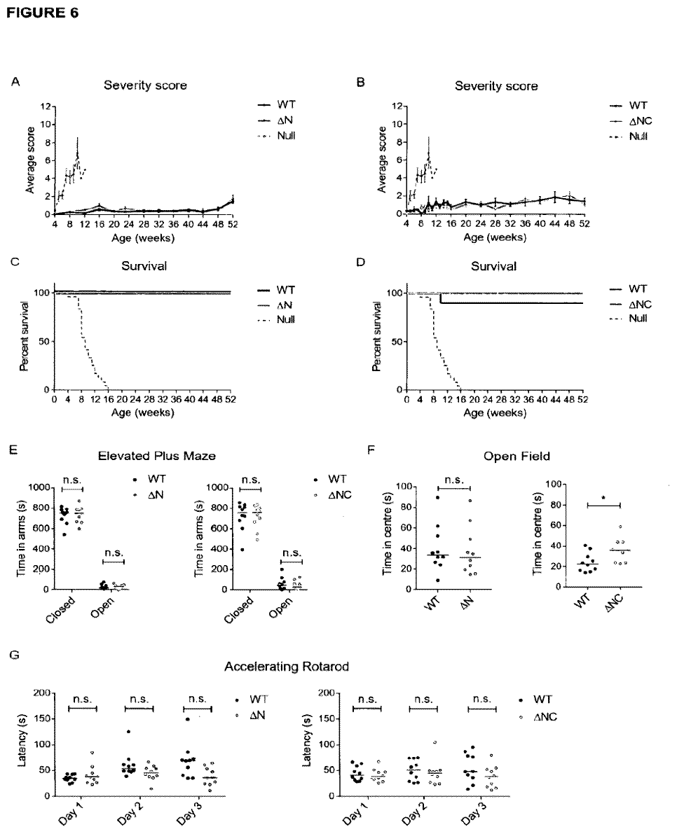

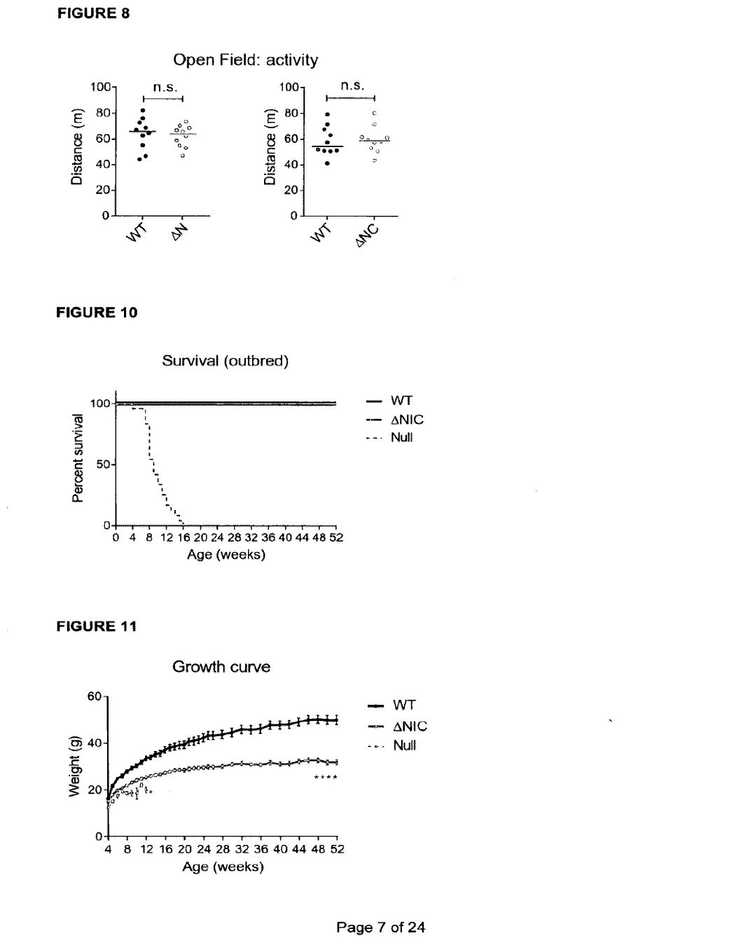

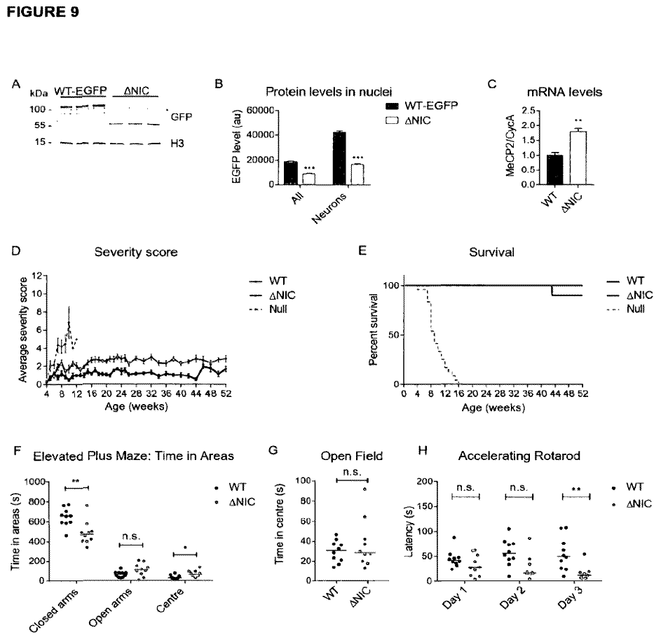

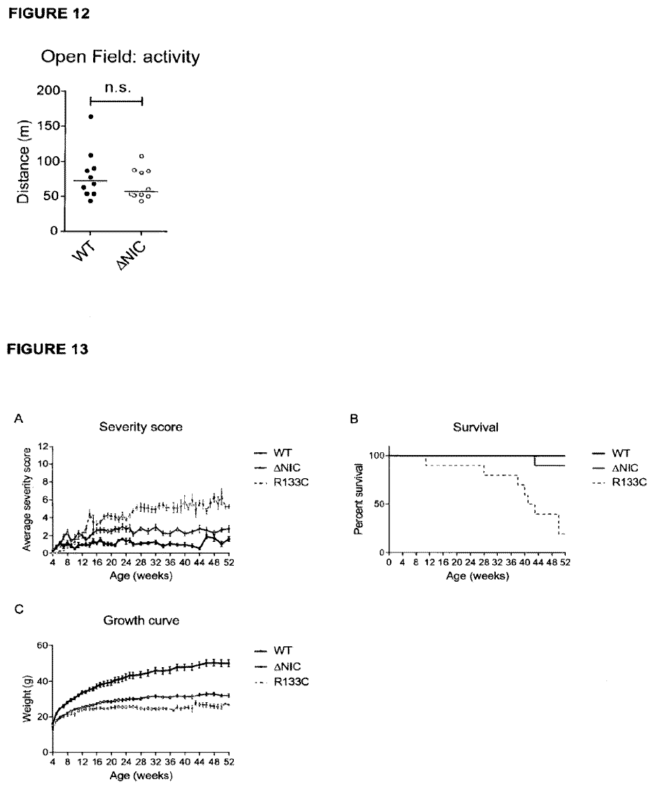

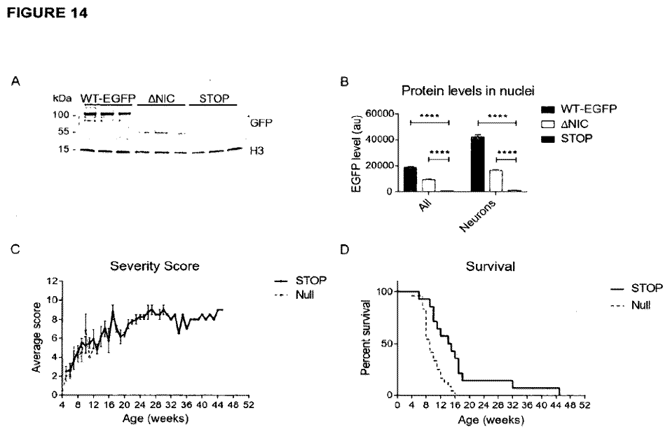

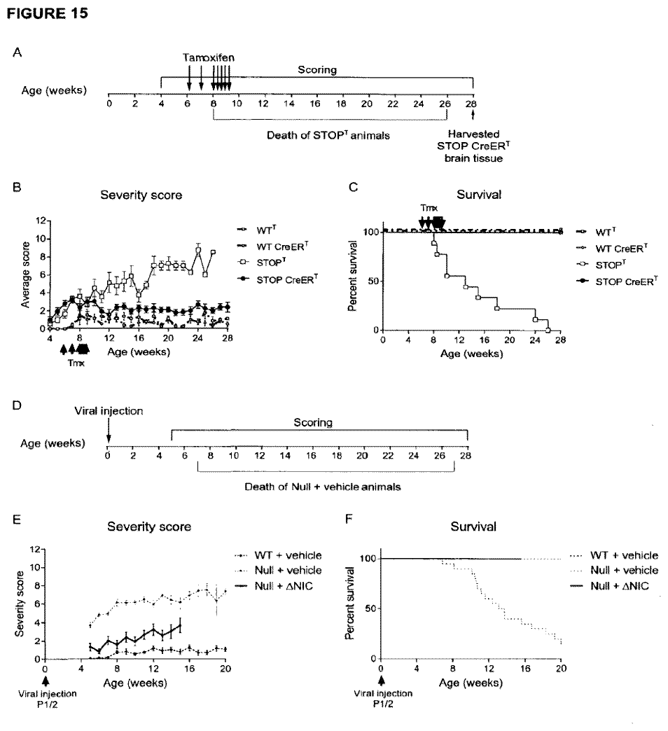

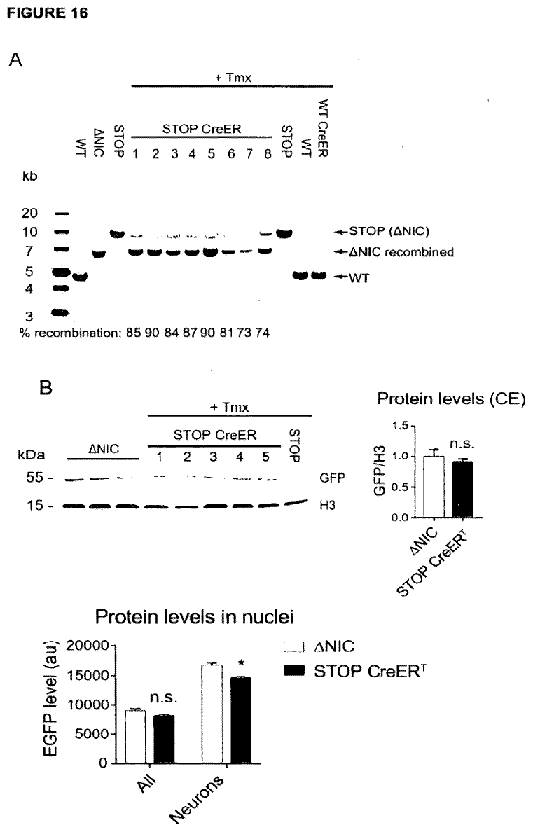

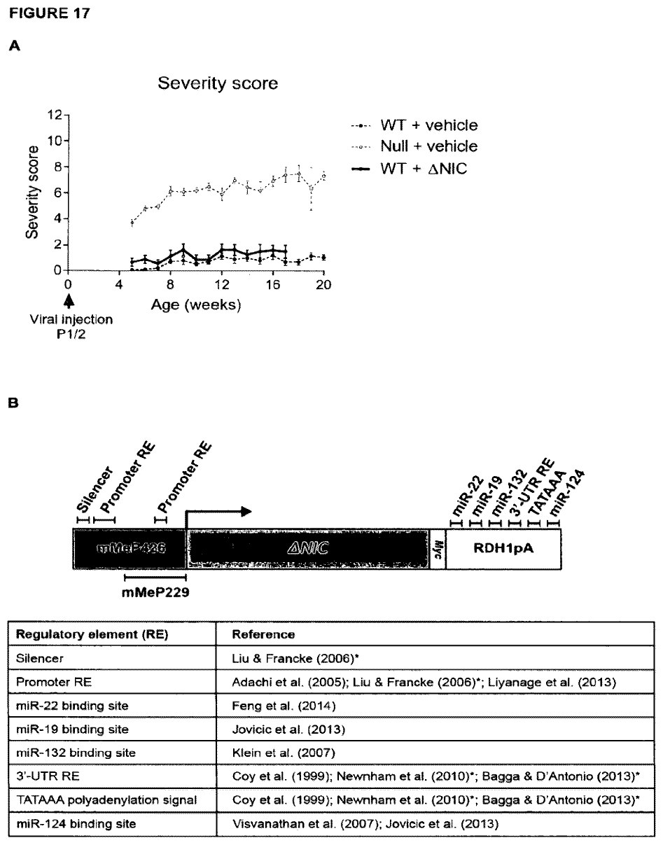

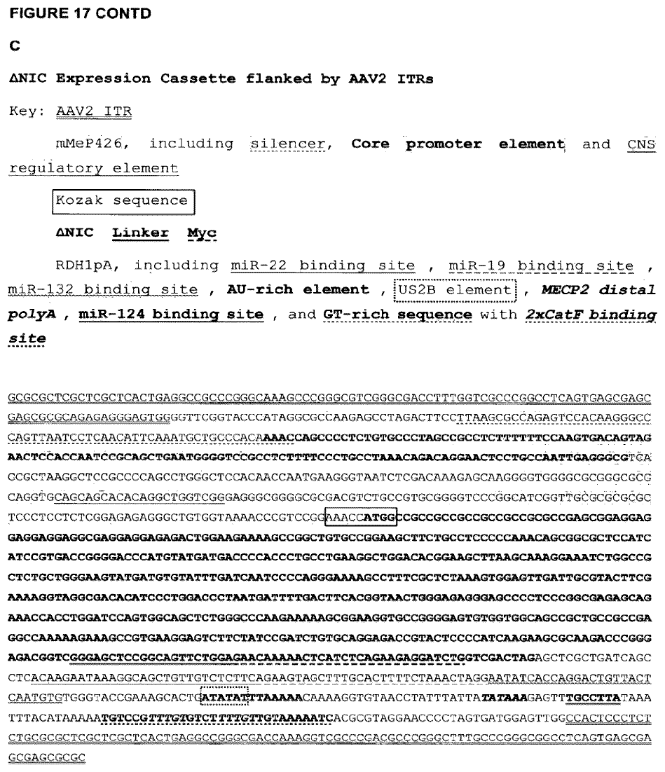

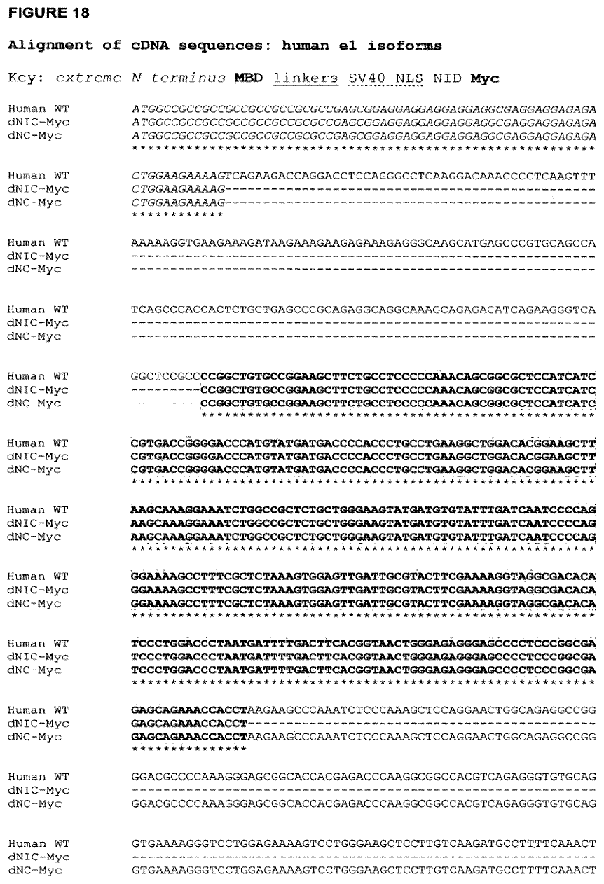

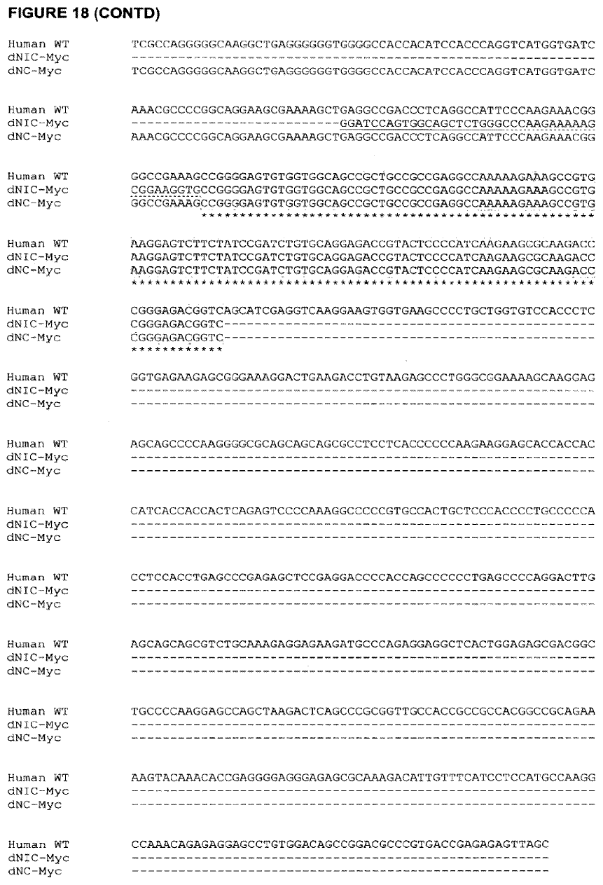

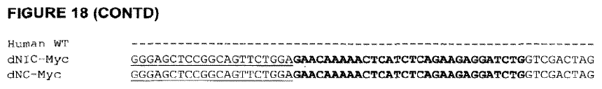

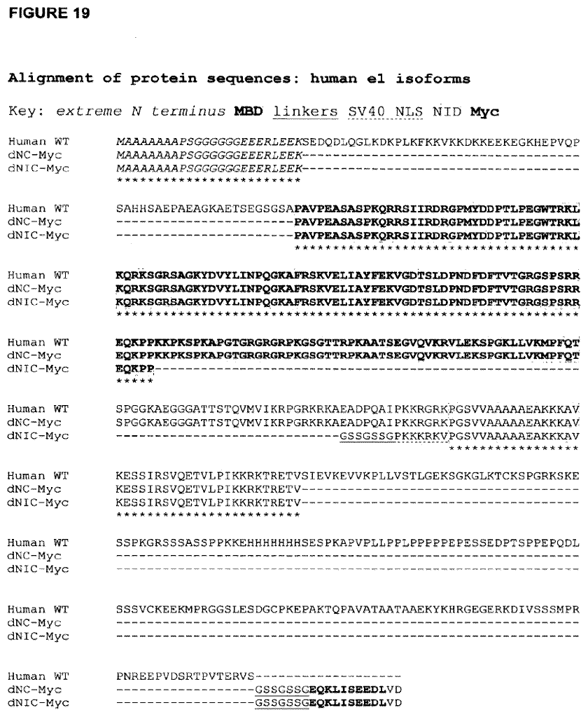

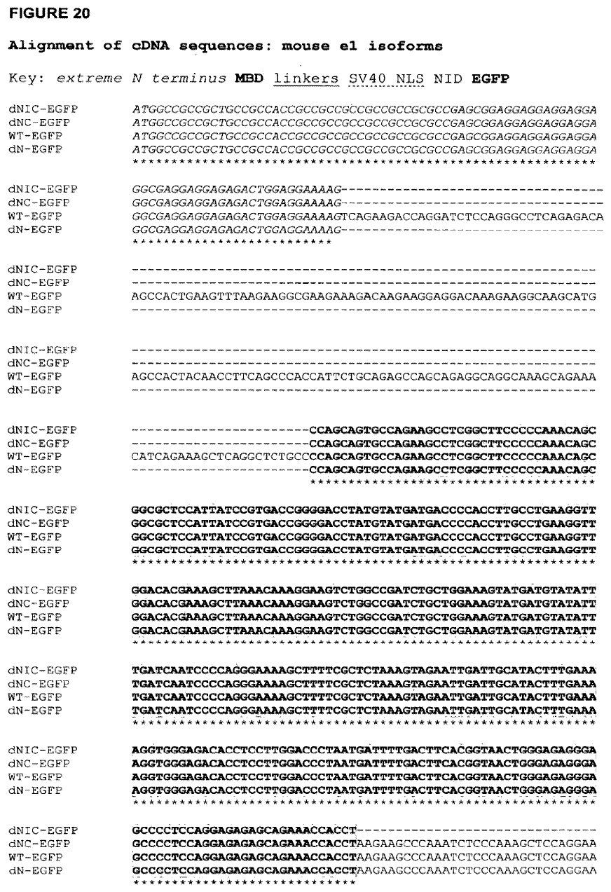

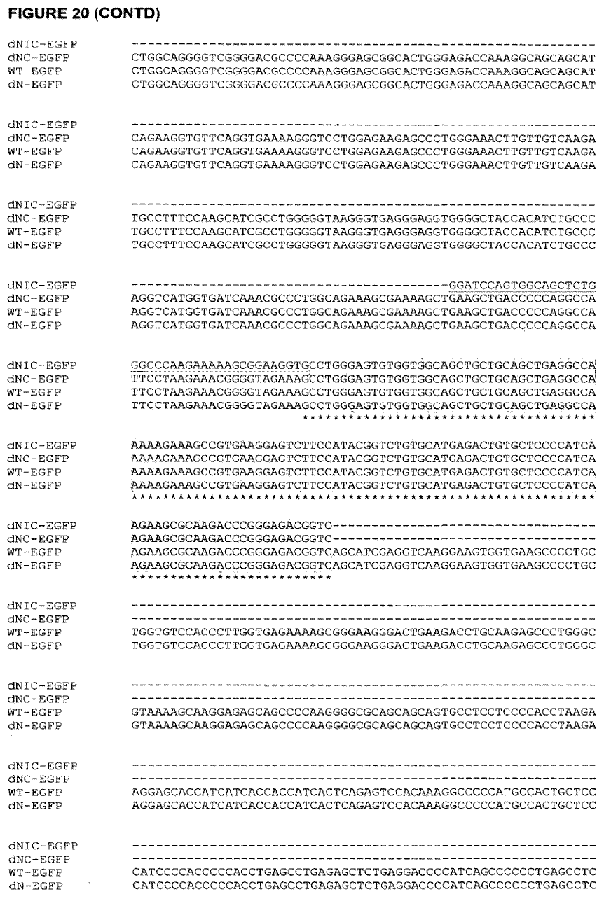

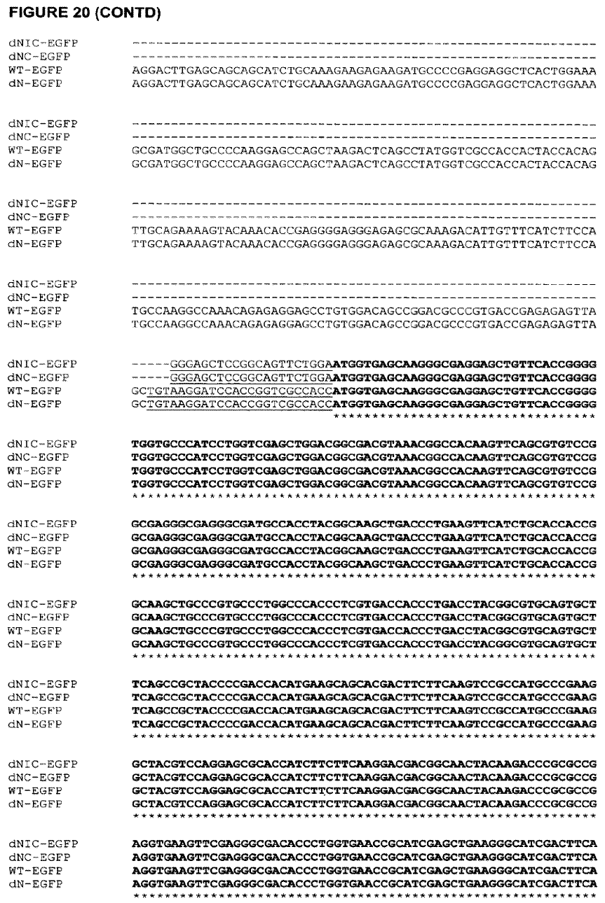

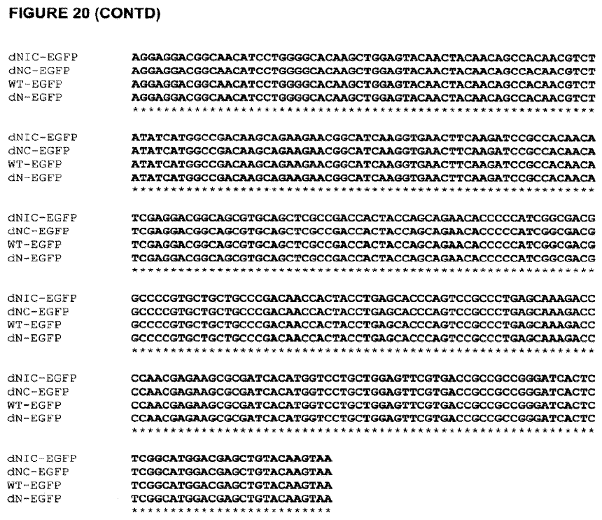

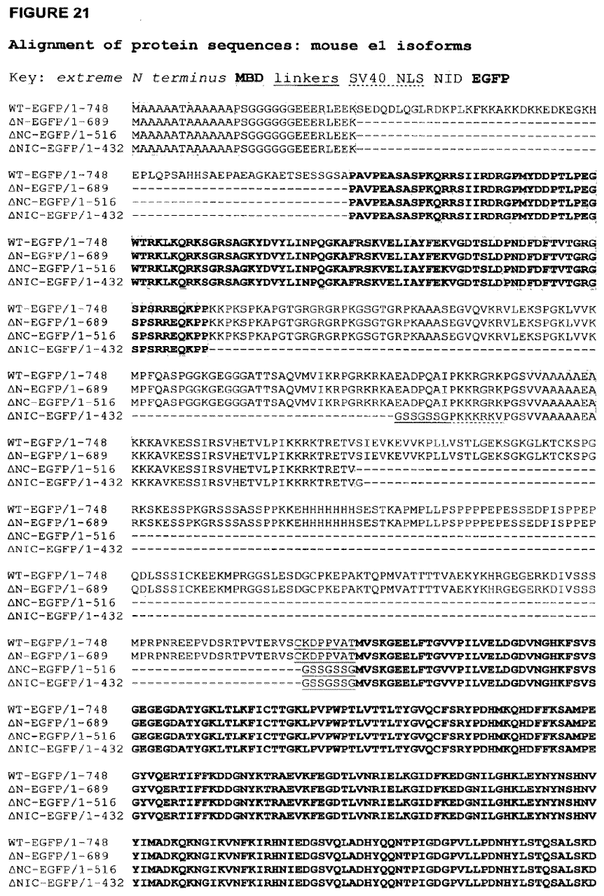

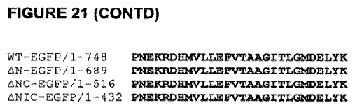

We initially generated ΔN and ΔNC knock-in mice by replacing the endogenous Mecp2 allele in ES cells followed by blastocyst injection and germ line transmission (FIG. 3). These truncated proteins were expressed at approximately wild-type levels in whole brain and in neurons as determined by western blot and flow cytometry analyses (FIG. 5A-B). To assess the phenotype of these truncations, knock-in mice were crossed onto a C57BL/6J background and cohorts underwent weekly phenotypic scoring^{24,27} or behavioural analysis. Both ΔN and ΔNC hemizygous male mice were viable, fertile and showed phenotypic scores indistinguishable from their wild-type littermates over the course of a year (FIG. 6A-D). ΔN mice had no body weight phenotype (FIG. 7A), whereas ΔNC mice displayed a slight increase in weight compared to wild-type littermates (FIG. 7B, repeated measures ΔNOVA p<0.0001). The weight difference was absent in a more outbred (75% C57BL/6J) cohort of ΔNC mice (FIG. 7C), consistent with previous observations that body weight phenotypes in RTT models are affected by genetic background²⁶.

At 20 weeks of age, separate cohorts were tested for behaviours commonly reported in RTT models: hypoactivity, decreased anxiety and reduced motor abilities. No activity phenotype (analysed by total distance travelled in the Open Field test) was detected for either the ΔN or ΔNC mice (FIG. 8). No anxiety phenotype (analysed by increased time spent in the open arms of the Elevated Plus Maze) was detected for either novel mouse line (FIG. 6E). The ΔNC mice did, however, spend significantly more time than their wild-type littermates in the central square of the Open Field arena (FIG. 6F), indicative of mildly decreased anxiety. Motor coordination was assessed using the Accelerating Rotarod test over three days. Whereas mouse models of RTT show impaired performance in this test that is most striking on the third day^{34,16}, ΔN and ΔNC mice were not significantly different from their wild-type littermates on any of the three days (FIG. 6G). Overall, the results suggest that contributions of the N- and C-terminal domains to MeCP2 function are at best subtle. This result is particularly remarkable given RTT-like symptoms in male mice expressing a slightly more severe C-terminal truncation, which lacks residues beyond T308³⁵. The difference in phenotype may be explained by retention of full NID function in ΔNC mice, as previous evidence indicates that loss of the further four C-terminal amino acids (309-312) reduces the affinity of this truncated MeCP2 molecule for the NCoR/SMRT co-repressor complex⁴.

We next replaced the endogenous Mecp2 gene with ΔNIC, the minimal allele, containing only the MBD and NID domains and comprising 32% of the full-length protein sequence (FIG. 1B, 4). Protein levels in whole brain were quantified by western blotting and flow cytometry, both of which showed reduced abundance (˜50% of WT-EGFP controls; FIG. 9A-B). A similar reduction in protein abundance was also seen in the neuronal subpopulation (˜40% of WT-EGFP controls; FIG. 9B). Low protein levels were not due to transcriptional silencing, as mRNA was in fact more abundant in ΔNIC mice than in wild-type littermates (FIG. 9C), suggesting that deletion of the intermediate region compromises protein stability. Despite low protein levels, male ΔNIC mice had a normal lifespan (FIG. 9E, 10). Phenotypic scoring over one year detected mild neurological phenotypes (FIG. 9D), predominantly gait abnormalities and partial hind-limb clasping. These symptoms persisted throughout the scoring period, but did not become more severe. ΔNIC mice also weighed ˜40% less than their wild-type siblings (FIG. 11A; repeated measures ΔNOVA p<0.0001). As seen this this study, both increases and decreases in body weight have been previously reported in MeCP2-mutant mouse models^{26,36,23,16}. Behavioural analysis of a separate cohort at 20 weeks showed decreased anxiety in male ΔNIC mice, as evidenced by the significantly reduced time spent in the closed arms of the Elevated Plus Maze (FIG. 9F, KS test p=0.003). This result was not supported by the Open Field test (FIG. 9G), which also detected no activity phenotype (FIG. 12). Consistent with the gait defects detected in the scoring cohort, ΔNIC mice had reduced motor coordination, shown by declining performance over three daily trials on the Accelerating Rotarod (FIG. 9H, Friedman test p=0.003). This resulted in significantly impaired performance on the third day of testing compared to wild-type littermates (KS test p=0.003). Overall, it is noteworthy that ΔNIC animals are much less severely affected than male mice with the mildest common mutation found in RTT patients, R133C, which had a median lifespan of 42 weeks, higher symptomatic scores and a stronger reduced weight phenotype¹⁶ (FIG. 13). This result strongly supports our hypothesis that recruitment of the NCoR/SMRT co-repressor complex to chromatin is the primary function of MeCP2, with the mild phenotype observed being a likely consequence of reduced protein levels, as previously described for hypomorphic mice that express full-length MeCP2 at 50% of wild-type levels³⁷.

To further test the functionality of minimal MeCP2, we asked whether late provision of ΔNIC via genetic reactivation could reverse phenotypic defects in symptomatic MeCP2-deficient mice, as has previously been shown for the full-length protein²⁴. We generated null-like MeCP2-deficient mice by preventing ΔNIC expression with a floxed transcriptional STOP cassette in intron 2 (FIG. 4, 14). These mice were crossed with mice carrying a CreER transgene (Cre recombinase fused to a modified estrogen receptor) to enable reactivation upon Tamoxifen treatment. This was induced after the onset of symptoms in STOP CreER mice (FIG. 15A), resulting in high levels of Cre recombination (FIG. 16A) and protein levels similar to ΔNIC mice (FIG. 16B). ΔNIC gene reactivation had a dramatic effect on phenotypic progression, ameliorating neurological symptoms and restoring normal survival (FIG. 15B-C). In contrast, STOP mice lacking the CreER transgene failed to survive beyond 26 weeks. Thus, despite its radically reduced length and relatively low abundance, ΔNIC was able to effectively reverse the RTT-like phenotype in MeCP2-deficient mice.

This finding prompted us to explore whether ΔNIC could be used for gene therapy, which we tested in Mecp2-null mice. The ΔNIC gene, driven by a minimal Mecp2 promoter, was tagged with a Myc epitope (in place of much larger EGFP) and packaged into an adeno-associated viral vector (AAV9). Neonatal mice (P1-2) were injected intra-cranially with this virus or the AAV vehicle alone (FIG. 15D). Mecp2-null animals receiving the ΔNIC gene showed greatly reduced symptom severity and enhanced survival (FIG. 15E-F). Despite the lack of fine control over infection rate per brain cell, we did not observe deleterious effects due to over-expression, even in wild-type animals (FIG. 17). It is conceivable that toxicity is mitigated by the moderate instability and/or reduced activity of ΔNIC protein. This experiment also shows that ΔNIC protein is functional without the large EGFP tag. The use of minimal MeCP2 could provide a therapeutic advantage due to the restricted capacity of AAV vectors. Shortening the coding sequence creates room for additional regulatory sequences, enabling better control of expression levels.

### 3. Discussion

Overall our results argue against the view that MeCP2 functions as a multifunctional hub, and instead support a simpler model whereby its predominant function is to recruit the NCoR/SMRT co-repressor complex to methylated sites on chromatin. It is noteworthy that the minimal MeCP2 protein (ΔNIC) is missing all or part of several domains that have been highlighted as potentially important, including the AT-hooks²³, several activity-dependent phosphorylation sites^{17,18}, an RNA binding motif⁶ and interaction sites for proteins implicated in micro-RNA processing⁹, splicing¹⁰ and chromatin remodelling⁸. Importantly, our discovery that these two domains are sufficient to restore neuronal function to MeCP2-deficient mice has allowed us to show the therapeutic potential of the minimal protein.

### 4. Additional Experiment

The appearance of toxicity in the form of motor dysfunction, ataxia and apparent loss of proprioception when full length Mecp2/MECP2 is delivered to mice has been reported previously^{49, 50}. An independent report has recently shown an identical stereotyped ataxia and loss of proprioception in response to delivery of the AAV9 variant (AAVhu68) in larger mammalian species and has shown that the peripheral nerve dorsal root ganglia may be especially susceptible to AAV9 variant dosing⁵¹.

We have performed a direct comparison of full length MECP2 and the ΔNIC MECP2 (Table 3). We observed a significant reduction of ataxia and proprioception dysfunction in the AAV9 ΔNIC MECP2-treated animals compared to mice treated with full length MECP2. These data support the fact that, under identical conditions, the ΔNIC MECP2 minigene confers reduced susceptibility to known peripheral neurotoxicity compared to full length MeCP2.

TABLE 3
Comparison of full length MECP2 and the ΔNIC MECP2 AAV9-
MeP229/MECP2 refers to a AAV2/9 vector having the strong
229 bp fragment of endogenous Mecp2 promoter and full length
MECP2; AAV9-MeP426/MECP2 refers to a AAV2/9 vector having
the 426 bp fragment of endogenous Mecp2 promoter and full
length MECP2; AAV9-MeP426/ΔNIC MECP2 refers to a AAV2/9
vector having the 426 bp fragment of endogenous Mecp2 promoter
and the ΔNIC MECP2 minigene insert.

| Vector | Toxicity |
|---|---|
| AAV9-MeP229/MECP2 | Severe ataxia/loss of proprioception in |
| | 100% of treated mice |
| AAV9-MeP426/MECP2 | Mild ataxia/loss of proprioception/clasping |
| | in 100% of treated mice |
| AAV9-MeP426/ΔNIC | Mild ataxia/loss of proprioception/clasping |
| MECP2 | in <25% (4 of 17) of treated mice |

### REFERENCES

1. Kinde, B. et al. DNA methylation in the gene body influences MeCP2-mediated gene repression. Proc. Natl. Acad. Sci. U.S.A 113, 15114-15119 (2016)
2. Adams, V H et al. Intrinsic Disorder and Autonomous Domain Function in the Multifunctional Nuclear Protein, MeCP2. J. Biol. Chem. 282, 15057-64 (2007)
3. Lyst, M. J. & Bird, A. Rett syndrome: a complex disorder with simple roots. Nat. Rev. Genet. 16, 261-274 (2015).
4. Lyst, M. J. et al. Rett syndrome mutations abolish the interaction of MeCP2 with the NCoR/SMRT co-repressor. Nat. Neurosci. 16, 898-902 (2013).
5. Chahrour, M. et al. MeCP2, a key contributor to neurological disease, activates and represses transcription. Science 320, 1224-9 (2008).
6. Jeffery, L. & Nakielny, S. Components of the DNA methylation system of chromatin control are RNA-binding proteins. J. Biol. Chem. 279, 49479-49487 (2004).
7. Nan, X. et al. Interaction between chromatin proteins MECP2 and ATRX is disrupted by mutations that cause inherited mental retardation. Proc. Natl. Acad. Sci. U.S.A 104, 2709-14 (2007).
8. Agarwal, N. et al. MeCP2 interacts with HP1 and modulates its heterochromatin association during myogenic differentiation. Nucleic Acids Res. 35, 5402-8 (2007).
9. Cheng, T.-L. et al. MeCP2 suppresses nuclear microRNA processing and dendritic growth by regulating the DGCR8/Drosha complex. Dev. Cell 28, 547-60 (2014).
10. Young, J. I. et al. Regulation of RNA splicing by the methylation-dependent transcriptional repressor methyl-CpG binding protein 2. Proc. Natl. Acad. Sci. U.S.A 102, 17551-8 (2005).
11. Ragione, F. Della, Vacca, M., Fioriniello, S., Pepe, G. & Esposito, M. D. MECP2, a multi-talented modulator of chromatin architecture. Brief. Funct. Genomics 15, 1-12 (2016).
12. Nan, X., Meehan, R. R. & Bird, A. Dissection of the methyl-CpG binding domain from the chromosomal protein MeCP2. Nucleic Acids Res. 21, 4886-4892 (1993).
13. RettBase: Rett Syndrome Variation Database. at <http://mecp2.chw.edu.au/>
14. Exome Aggregation Consortium (ExAC), Cambridge, MA. at http://exac.broadinstitute.org
15. Dinca, A et al Intracellular delivery of proteins with cell-penetrating peptides for therapeutic uses in human disease. Int J Mol Sci. 17(2): 263 (2016).
16. Brown, K. et al. The molecular basis of variable phenotypic severity among common missense mutations causing Rett syndrome. Hum. Mol. Genet. 25, 558-570 (2016).
17. Zhou, Z. et al. Brain-Specific Phosphorylation of MeCP2 Regulates Activity-Dependent Bdnf Transcription, Dendritic Growth, and Spine Maturation. Neuron 52, 255-269 (2006).
18. Tao, J. et al. Phosphorylation of MeCP2 at Serine 80 regulates its chromatin association and neurological function. Proc. Natl. Acad. Sci. U.S.A 106, 4882-7 (2009).
19. Ebert, D. H. et al. Activity-dependent phosphorylation of MeCP2 threonine 308 regulates interaction with NCoR. Nature 499, 341-5 (2013).
20. Ho, K. L. et al. MeCP2 binding to DNA depends upon hydration at methyl-CpG. Mol. Cell 29, 525-31 (2008).
21. PHD Secondary structure prediction method. at <https://npsa-prabi.ibcp.fr/cgi-bin/npsa_automat.pl?page=/NPSA/npsa_phd.html>
22. Lyst, M. J., Connelly, J., Merusi, C. & Bird, A. Sequence-specific DNA binding by AT-hook motifs in MeCP2. FEBS Lett. 590, 2927-2933 (2016).
23. Baker, S. A. et al. An AT-hook domain in MeCP2 determines the clinical course of Rett syndrome and related disorders. Cell 152, 984-96 (2013).
24. Guy, J., Gan, J., Selfridge, J., Cobb, S. & Bird, A. Reversal of neurological defects in a mouse model of Rett syndrome. Science 315, 1143-7 (2007).
25. Cong, L. et al. Multiplex Genome Engineering Using CRISPR/VCas Systems. Science (80-.). 339, 819-823 (2013).
26. Guy, J., Hendrich, B., Holmes, M., Martin, J. E. & Bird, a. A mouse Mecp2-null mutation causes neurological symptoms that mimic Rett syndrome. Nat. Genet. 27, 322-6 (2001).
27. Cheval, H. et al. Postnatal inactivation reveals enhanced requirement for MeCP2 at distinct age windows. Hum. Mol. Genet. 21, 3806-3814 (2012).
28. Clément, N. & Grieger, J. C. Manufacturing of recombinant adeno-associated viral vectors for clinical trials. Mol. Ther. Methods Clin. Dev. 3, 16002 (2016).
29. Gadalla, K. K. E. et al. Improved survival and reduced phenotypic severity following AAV9/MECP2 gene transfer to neonatal and juvenile male Mecp2 knockout mice. Mol. Ther. 21, 18-30 (2013).
30. Kriaucionis, S. & Bird, A. The major form of MeCP2 has a novel N-terminus generated by alternative splicing. Nucleic Acids Res. 32, 1818-23 (2004).
31. Lewis, J. D. et al. Purification, sequence, and cellular localization of a novel chromosomal protein that binds to methylated DNA. Cell 69, 905-14 (1992).
32. Nan, X., Tate, P., Li, E. & Bird, A. DNA methylation specifies chromosomal localization of MeCP2. Mol. Cell. Biol. 16, 414-21 (1996).
33. Kudo, S. et al. Heterogeneity in residual function of MeCP2 carrying missense mutations in the methyl CpG binding domain. J. Med. Genet. 40, 487-93 (2003).
34. Goffin, D. et al. Rett syndrome mutation MeCP2 T158A disrupts DNA binding, protein stability and ERP responses. Nat. Neurosci. 15, 274-83 (2012).
35. Shahbazian, M. et al. Mice with truncated MeCP2 recapitulate many Rett syndrome features and display hyperacetylation of histone H3. Neuron 35, 243-54 (2002).
36. Chen, R. Z., Akbarian, S., Tudor, M. & Jaenisch, R. Deficiency of methyl-CpG binding protein-2 in CNS neurons results in a Rett-like phenotype in mice. Nat. Genet. 27, 327-331 (2001).
37. Samaco, R. C. et al. A partial loss of function allele of Methyl-CpG-binding protein 2 predicts a human neurodevelopmental syndrome. Hum. Mol. Genet. 17, 1718-1727 (2008).
38. Gray, S J, Foti, S B, Schwartz, J W, Bachaboina, L, Taylor-Blake, B, Coleman, J, et al. (2011). Optimizing promoters for recombinant adeno-associated virus-mediated gene expression in the peripheral and central nervous system using self-complementary vectors. Hum Gene Ther 22: 1143-1153.
39. Feng, Y, Huang, W, Wani, M, Yu, X, and Ashraf, M (2014). Ischemic preconditioning potentiates the protective effect of stem cells through secretion of exosomes by targeting Mecp2 via miR-22. PLoS One 9: e88685.
40. Jovicic, A, Roshan, R, Moisoi, N, Pradervand, S, Moser, R, Pillai, B, et al. (2013). Comprehensive expression analyses of neural cell-type-specific miRNAs identify new determinants of the specification and maintenance of neuronal phenotypes. J Neurosci 33: 5127-5137.
41. Klein, M E, Lioy, DT, Ma, L, Impey, S, Mandel, G, and Goodman, R H (2007). Homeostatic regulation of MeCP2 expression by a CREB-induced microRNA. Nat Neurosci 10: 1513-1514.
42. Liu, J, and Francke, U (2006). Identification of cis-regulatory elements for MECP2 expression. Human molecular genetics 15: 1769-1782.
43. Adachi, M, Keefer, E W, and Jones, F S (2005). A segment of the Mecp2 promoter is sufficient to drive expression in neurons. Human molecular genetics 14: 3709-3722.
44. Liyanage, VR, Zachariah, R M, and Rastegar, M (2013). Decitabine alters the expression of Mecp2 isoforms via dynamic DNA methylation at the Mecp2 regulatory elements in neural stem cells. Molecular autism 4: 46.
45. Visvanathan, J, Lee, S, Lee, B, Lee, J W, and Lee, SK (2007). The microRNA miR-124 antagonizes the anti-neural REST/SCP1 pathway during embryonic CNS development. Genes Dev 21: 744-749.
46. Coy, J F, Sedlacek, Z, Bachner, D, Delius, H, and Poustka, A (1999). A complex pattern of evolutionary conservation and alternative polyadenylation within the long 3″-untranslated region of the methyl-CpG-binding protein 2 gene (MeCP2) suggests a regulatory role in gene expression. Human molecular genetics 8: 1253-1262.
47. Bagga, J S, and D'Antonio, L A (2013). Role of conserved cis-regulatory elements in the post-transcriptional regulation of the human MECP2 gene involved in autism. Human genomics 7: 19.
48. Newnham, CM, Hall-Pogar, T, Liang, S, Wu, J, Tian, B, Hu, J, et al. (2010). Alternative polyadenylation of MeCP2: Influence of cis-acting elements and trans-acting factors. RNA biology 7: 361-372.
49. Gadalla, K. (2012) Virus-mediated delivery of MECP2 as a potential tool for the treatment of Rett syndrome. PhD thesis, http://theses.gla.ac.uk/id/eprint/3501
50. Gadalla, K. K. E., Vudhironarit, T., Hector, R. D., Sinnett, S., Bahey, N. G., Bailey, M. E. S., Gray, S. J., Cobb, S. R. (2017) Development of a Novel AAV Gene Therapy Cassette with Improved Safety Features and Efficacy in a Mouse Model of Rett Syndrome. Mol Ther Methods Clin Dev. 5:180-190. doi: 10.1016/j.omtm.2017.04.007.
51. Hinderer, C., Katz, N., Buza, E. L., Dyer, C., Goode, T., Bell, P., Richman, L. K., Wilson, J. M. (2018) Severe Toxicity in Nonhuman Primates and Piglets Following High-Dose Intravenous Administration of an Adeno-Associated Virus Vector Expressing Human SMN. Hum Gene Ther. doi: 10.1089/hum.2018.015.

1.-28. (canceled)

29. A pharmaceutical composition comprising:
an expression vector comprising a MECP2 expression cassette comprising a MecP2 gene, wherein the MecP2 gene comprises a deletion of at least 50 amino acids compared to the full length human MeCP2 e1 and e2 sequences (SEQ ID NOs 3 and 4) and
a pharmaceutically acceptable carrier.

30. The pharmaceutical composition of claim 1, wherein the expression cassette further comprises two ITR sequences.

31. The pharmaceutical composition of claim 2, wherein the two ITR sequences are AAV2 ITR sequences.

32. The pharmaceutical composition of claim 1, wherein the vector is an AAV9 vector.

33. The pharmaceutical composition of claim 1, wherein the pharmaceutically acceptable carrier is a diluent, solubilizing or emulsifying agent, or a salt.

34. The pharmaceutical composition of claim 1, wherein the pharmaceutically acceptable carrier is a buffered saline solution.

35. The pharmaceutical composition of claim 1, wherein the pharmaceutically acceptable carrier is phosphate buffered saline solution.

36. The pharmaceutical composition of claim 1, wherein the formulation has pH of 7.0-8.0.

37. A method of treating a neurological disorder associated with inactivating mutation of MeCP2 in a subject in need thereof, comprising administering to the subject a pharmaceutical composition comprising an expression vector comprising a MECP2 expression cassette comprising a MecP2 gene, wherein the MecP2 gene comprises a deletion of at least 50 amino acids compared to the full length human MeCP2 e1 and e2 sequences (SEQ ID NOs 3 and 4) and a pharmaceutically acceptable carrier.

38. The method of claim 37, wherein the pharmaceutical composition is administered to the central nervous system (“CNS”).

39. The method of claim 37, wherein the neurological disorder is Rett Syndrome.


SEQUENCE LISTING (71 sequences)

SEQ ID NO: 1 - protein, 102 aa, Homo sapiens
PAVPEASASPKQRRSIIRDRGPMYDDPTLPEGWTRKLKQRKSGRSAGKYDVYLINPQGKAFRSKVELIAYFEKVGDTSLDPNDFDFTVTGRGSPSRREQKPP

SEQ ID NO: 2 - protein, 41 aa, Homo sapiens
PGSVVAAAAAEAKKKAVKESSIRSVQETVLPIKKRKTRETV

SEQ ID NO: 3 - protein, 498 aa, Homo sapiens [sequence omitted]

SEQ ID NO: 4 - protein, 486 aa, Homo sapiens
MVAGMLGLREEKSEDQDLQGLKDKPLKFKKVKKDKKEEKEGKHEPVQPSAHHSAEPAEAGKAETSEGSGSAPAVPEASASPKQRRSIIRDRGPMYDDPTLPEGWTRKLKQRKSGRSAGKYDVYLINPQGKAFRSKVELIAYFEKVGDTSLDPNDFDFTVTGRGSPSRREQKPPKKPKSPKAPGTGRGRGRPKGSGTTRPKAATSEGVQVKRVLEKSPGKLLVKMPFQTSPGGKAEGGGATTSTQVMVIKRPGRKRKAEADPQAIPKKRGRKPGSVVAAAAAEAKKKAVKESSIRSVQETVLPIKKRKTRETVSIEVKEVVKPLLVSTLGEKSGKGLKTCKSPGRKSKESSPKGRSSSASSPPKKEHHHHHHHSESPKAPVPLLPPLPPPPPEPESSEDPTSPPEPQDLSSSVCKEEKMPRGGSLESDGCPKEPAKTQPAVATAATAAEKYKHRGEGERKDIVSSSMPRPNREEPVDSRTPVTERVS

SEQ ID NO: 5 - protein, 83 aa, Homo sapiens
MAAAAAAAPSGGGGGGEEERLEEKSEDQDLQGLKDKPLKFKKVKKDKKEEKEGKHEPVQPSAHHSAEPAEAGKAETSEGSGSA

SEQ ID NO: 6 - protein, 71 aa, Homo sapiens
MVAGMLGLREEKSEDQDLQGLKDKPLKFKKVKKDKKEEKEGKHEPVQPSAHHSAEPAEAGKAETSEGSGSA

SEQ ID NO: 7 - protein, 98 aa, Homo sapiens
KKPKSPKAPGTGRGRGRPKGSGTTRPKAATSEGVQVKRVLEKSPGKLLVKMPFQTSPGGKAEGGGATTSTQVMVIKRPGRKRKAEADPQAIPKKRGRK

SEQ ID NO: 8 - protein, 174 aa, Homo sapiens
SIEVKEVVKPLLVSTLGEKSGKGLKTCKSPGRKSKESSPKGRSSSASSPPKKEHHHHHHHSESPKAPVPLLPPLPPPPPEPESSEDPTSPPEPQDLSSSVCKEEKMPRGGSLESDGCPKEPAKTQPAVATAATAAEKYKHRGEGERKDIVSSSMPRPNREEPVDSRTPVTERVS

SEQ ID NO: 9 - protein, 29 aa, Mus musculus
MAAAAATAAAAAAPSGGGGGGEEERLEEK

SEQ ID NO: 10 - protein, 12 aa, Mus musculus
MVAGMLGLREEK

SEQ ID NO: 11 - protein, 24 aa, Homo sapiens
MAAAAAAAPSGGGGGGEEERLEEK

SEQ ID NO: 12 - protein, 12 aa, Homo sapiens
MVAGMLGLREEK

SEQ ID NO: 13 - protein, 241 aa, synthetic construct
  REGION 1..241: note = dNC: A truncated synthetic polypeptide sequence (from human MeCP2)
PAVPEASASPKQRRSIIRDRGPMYDDPTLPEGWTRKLKQRKSGRSAGKYDVYLINPQGKAFRSKVELIAYFEKVGDTSLDPNDFDFTVTGRGSPSRREQKPPKKPKSPKAPGTGRGRGRPKGSGTTRPKAATSEGVQVKRVLEKSPGKLLVKMPFQTSPGGKAEGGGATTSTQVMVIKRPGRKRKAEADPQAIPKKRGRKPGSVVAAAAAEAKKKAVKESSIRSVQETVLPIKKRKTRETV

SEQ ID NO: 14 - protein, 157 aa, synthetic construct
  REGION 1..157: note = dNIC: A truncated synthetic polypeptide sequence (from human MeCP2)
PAVPEASASPKQRRSIIRDRGPMYDDPTLPEGWTRKLKQRKSGRSAGKYDVYLINPQGKAFRSKVELIAYFEKVGDTSLDPNDFDFTVTGRGSPSRREQKPPGSSGSSGPKKKRKVPGSVVAAAAAEAKKKAVKESSIRSVQETVLPIKKRKTRETV

SEQ ID NO: 15 - protein, 413 aa, synthetic construct
  REGION 1..413: note = dN mouse: A truncated synthetic polypeptide sequence (from mouse MeCP2)
PAVPEASASPKQRRSIIRDRGPMYDDPTLPEGWTRKLKQRKSGRSAGKYDVYLINPQGKAFRSKVELIAYFEKVGDTSLDPNDFDFTVTGRGSPSRREQKPPKKPKSPKAPGTGRGRGRPKGSGTGRPKAAASEGVQVKRVLEKSPGKLVVKMPFQASPGGKGEGGGATTSAQVMVIKRPGRKRKAEADPQAIPKKRGRKPGSVVAAAAAEAKKKAVKESSIRSVHETVLPIKKRKTRETVSIEVKEVVKPLLVSTLGEKSGKGLKTCKSPGRKSKESSPKGRSSSASSPPKKEHHHHHHHSESTKAPMPLLPSPPPPEPESSEDPISPPEPQDLSSSICKEEKMPRGGSLESDGCPKEPAKTQPMVATTTTVAEKYKHRGEGERKDIVSSSMPRPNREEPVDSRTPVTERVS

SEQ ID NO: 16 - protein, 241 aa, synthetic construct
  REGION 1..241: note = dNC mouse: A truncated synthetic polypeptide sequence (from mouse MeCP2)
PAVPEASASPKQRRSIIRDRGPMYDDPTLPEGWTRKLKQRKSGRSAGKYDVYLINPQGKAFRSKVELIAYFEKVGDTSLDPNDFDFTVTGRGSPSRREQKPPKKPKSPKAPGTGRGRGRPKGSGTGRPKAAASEGVQVKRVLEKSPGKLVVKMPFQASPGGKGEGGGATTSAQVMVIKRPGRKRKAEADPQAIPKKRGRKPGSVVAAAAAEAKKKAVKESSIRSVHETVLPIKKRKTRETV

SEQ ID NO: 17 - protein, 157 aa, synthetic construct
  REGION 1..157: note = dNIC mouse: A truncated synthetic polypeptide sequence (from mouse MeCP2)
PAVPEASASPKQRRSIIRDRGPMYDDPTLPEGWTRKLKQRKSGRSAGKYDVYLINPQGKAFRSKVELIAYFEKVGDTSLDPNDFDFTVTGRGSPSRREQKPPGSSGSSGPKKKRKVPGSVVAAAAAEAKKKAVKESSIRSVHETVLPIKKRKTRETV

SEQ ID NO: 18 - DNA, 1494 bp, Homo sapiens
  source 1..1494: mol_type = genomic DNA
atggccgccgccgccgccgccgcgccgagcggaggaggaggaggaggcgaggaggagagactggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaatccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagcgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagc

SEQ ID NO: 19 - DNA, 723 bp, synthetic construct
  misc_feature 1..723: note = dNC: cDNA sequence of a truncated synthetic polypeptide sequence (from human MeCP2)
  source 1..723: mol_type = other DNA
ccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaatccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtc

SEQ ID NO: 20 - DNA, 471 bp, synthetic construct
  misc_feature 1..471: note = dNIC: cDNA sequence of a truncated synthetic polypeptide sequence (from human MeCP2)
  source 1..471: mol_type = other DNA
ccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaatccccagggaaaagcctttcgctctaaagtggagttgattgcgtacttcgaaaaggtaggcgacacatccctggaccctaatgattttgacttcacggtaactgggagagggagcccctcccggcgagagcagaaaccacctggatccagtggcagctctgggcccaagaaaaagcggaaggtgccggggagtgtggtggcagccgctgccgccgaggccaaaaagaaagccgtgaaggagtcttctatccgatctgtgcaggagaccgtactccccatcaagaagcgcaagacccgggagacggtc

SEQ ID NO: 21 - DNA, 1506 bp, Mus musculus
  source 1..1506: mol_type = genomic DNA
atggccgccgctgccgccaccgccgccgccgccgccgcgccgagcggaggaggaggaggaggcgaggaggagagactggaggaaaagtcagaagaccaggatctccagggcctcagagacaagccactgaagtttaagaaggcgaagaaagacaagaaggaggacaaagaaggcaagcatgagccactacaaccttcagcccaccattctgcagagccagcagaggcaggcaaagcagaaacatcagaaagctcaggctctgccccagcagtgccagaagcctcggcttcccccaaacagcggcgctccattatccgtgaccggggacctatgtatgatgaccccaccttgcctgaaggttggacacgaaagcttaaacaaaggaagtctggccgatctgctggaaagtatgatgtatatttgatcaatccccagggaaaagcttttcgctctaaagtagaattgattgcatactttgaaaaggtgggagacacctccttggaccctaatgattttgacttcacggtaactgggagagggagcccctccaggagagagcagaaaccacctaagaagcccaaatctcccaaagctccaggaactggcaggggtcggggacgccccaaagggagcggcactgggagaccaaaggcagcagcatcagaaggtgttcaggtgaaaagggtcctggagaagagccctgggaaacttgttgtcaagatgcctttccaagcatcgcctgggggtaagggtgagggaggtggggctaccacatctgcccaggtcatggtgatcaaacgccctggcagaaagcgaaaagctgaagctgacccccaggccattcctaagaaacggggtagaaagcctgggagtgtggtggcagctgctgcagctgaggccaaaaagaaagccgtgaaggagtcttccatacggtctgtgcatgagactgtgctccccatcaagaagcgcaagacccgggagacggtcagcatcgaggtcaaggaagtggtgaagcccctgctggtgtccacccttggtgagaaaagcgggaagggactgaagacctgcaagagccctgggcgtaaaagcaaggagagcagccccaaggggcgcagcagcagtgcctcctccccacctaagaaggagcaccatcatcaccaccatcactcagagtccacaaaggcccccatgccactgctcccatccccacccccacctgagcctgagagctctgaggaccccatcagcccccctgagcctcaggacttgagcagcagcatctgcaaagaagagaagatgccccgaggaggctcactggaaagcgatggctgccccaaggagccagctaagactcagcctatggtcgccaccactaccacagttgcagaaaagtacaaacaccgaggggagggagagcgcaaagacattgtttcatcttccatgccaaggccaaacagagaggagcctgtggacagccggacgcccgtgaccgagagagttagctct

SEQ ID NO: 22 - DNA, 1242 bp, synthetic construct
  misc_feature 1..1242: note = dN mouse: cDNA for a truncated synthetic polypeptide sequence (from mouse MeCP2)
  source 1..1242: mol_type = other DNA
ccagcagtgccagaagcctcggcttcccccaaacagcggcgctccattatccgtgaccggggacctatgtatgatgaccccaccttgcctgaaggttggacacgaaagcttaaacaaaggaagtctggccgatctgctggaaagtatgatgtatatttgatcaatccccagggaaaagcttttcgctctaaagtagaattgattgcatactttgaaaaggtgggagacacctccttggaccctaatgattttgacttcacggtaactgggagagggagcccctccaggagagagcagaaaccacctaagaagcccaaatctcccaaagctccaggaactggcaggggtcggggacgccccaaagggagcggcactgggagaccaaaggcagcagcatcagaaggtgttcaggtgaaaagggtcctggagaagagccctgggaaacttgttgtcaagatgcctttccaagcatcgcctgggggtaagggtgagggaggtggggctaccacatctgcccaggtcatggtgatcaaacgccctggcagaaagcgaaaagctgaagctgacccccaggccattcctaagaaacggggtagaaagcctgggagtgtggtggcagctgctgcagctgaggccaaaaagaaagccgtgaaggagtcttccatacggtctgtgcatgagactgtgctccccatcaagaagcgcaagacccgggagacggtcagcatcgaggtcaaggaagtggtgaagcccctgctggtgtccacccttggtgagaaaagcgggaagggactgaagacctgcaagagccctgggcgtaaaagcaaggagagcagccccaaggggcgcagcagcagtgcctcctccccacctaagaaggagcaccatcatcaccaccatcactcagagtccacaaaggcccccatgccactgctcccatccccacccccacctgagcctgagagctctgaggaccccatcagcccccctgagcctcaggacttgagcagcagcatctgcaaagaagagaagatgccccgaggaggctcactggaaagcgatggctgccccaaggagccagctaagactcagcctatggtcgccaccactaccacagttgcagaaaagtacaaacaccgaggggagggagagcgcaaagacattgtttcatcttccatgccaaggccaaacagagaggagcctgtggacagccggacgcccgtgaccgagagagttagctgt

SEQ ID NO: 23 - DNA, 723 bp, synthetic construct
  misc_feature 1..723: note = dNC mouse: cDNA for a truncated synthetic polypeptide sequence (from mouse MeCP2)
  source 1..723: mol_type = other DNA
ccagcagtgccagaagcctcggcttcccccaaacagcggcgctccattatccgtgaccggggacctatgtatgatgaccccaccttgcctgaaggttggacacgaaagcttaaacaaaggaagtctggccgatctgctggaaagtatgatgtatatttgatcaatccccagggaaaagcttttcgctctaaagtagaattgattgcatactttgaaaaggtgggagacacctccttggaccctaatgattttgacttcacggtaactgggagagggagcccctccaggagagagcagaaaccacctaagaagcccaaatctcccaaagctccaggaactggcaggggtcggggacgccccaaagggagcggcactgggagaccaaaggcagcagcatcagaaggtgttcaggtgaaaagggtcctggagaagagccctgggaaacttgttgtcaagatgcctttccaagcatcgcctgggggtaagggtgagggaggtggggctaccacatctgcccaggtcatggtgatcaaacgccctggcagaaagcgaaaagctgaagctgacccccaggccattcctaagaaacggggtagaaagcctgggagtgtggtggcagctgctgcagctgaggccaaaaagaaagccgtgaaggagtcttccatacggtctgtgcatgagactgtgctccccatcaagaagcgcaagacccgggagacggtc

SEQ ID NO: 24 - DNA, 471 bp, synthetic construct
  misc_feature 1..471: note = dNIC mouse: cDNA for a truncated synthetic polypeptide sequence (from mouse MeCP2)
  source 1..471: mol_type = other DNA
ccagcagtgccagaagcctcggcttcccccaaacagcggcgctccattatccgtgaccggggacctatgtatgatgaccccaccttgcctgaaggttggacacgaaagcttaaacaaaggaagtctggccgatctgctggaaagtatgatgtatatttgatcaatccccagggaaaagcttttcgctctaaagtagaattgattgcatactttgaaaaggtgggagacacctccttggaccctaatgattttgacttcacggtaactgggagagggagcccctccaggagagagcagaaaccacctggatccagtggcagctctgggcccaagaaaaagcggaaggtgcctgggagtgtggtggcagctgctgcagctgaggccaaaaagaaagccgtgaaggagtcttccatacggtctgtgcatgagactgtgctccccatcaagaagcgcaagacccgggagacggtc

SEQ ID NO: 25 - DNA, 87 bp, Mus musculus
  source 1..87: mol_type = genomic DNA
atggccgccgctgccgccaccgccgccgccgccgccgcgccgagcggaggaggaggaggaggcgaggaggagagactggaggaaaag

SEQ ID NO: 26 - DNA, 46 bp, Mus musculus
  source 1..46: mol_type = genomic DNA
atggtagctgggatgttagggctcagggaggaaaagggaggaaaag

SEQ ID NO: 27 - DNA, 72 bp, Homo sapiens
  source 1..72: mol_type = genomic DNA
atggccgccgccgccgccgccgcgccgagcggaggaggaggaggaggcgaggaggagagactggaagaaaag

SEQ ID NO: 28 - DNA, 36 bp, Homo sapiens
  source 1..36: mol_type = genomic DNA
atggtagctgggatgttagggctcagggaagaaaag

SEQ ID NO: 29 - protein, 501 aa, Mus musculus
MAAAAATAAAAAAPSGGGGGGEEERLEEKSEDQDLQGLRDKPLKFKKAKKDKKEDKEGKHEPLQPSAHHSAEPAEAGKAETSESSGSAPAVPEASASPKQRRSIIRDRGPMYDDPTLPEGWTRKLKQRKSGRSAGKYDVYLINPQGKAFRSKVELIAYFEKVGDTSLDPNDFDFTVTGRGSPSRREQKPPKKPKSPKAPGTGRGRGRPKGSGTGRPKAAASEGVQVKRVLEKSPGKLVVKMPFQASPGGKGEGGGATTSAQVMVIKRPGRKRKAEADPQAIPKKRGRKPGSVVAAAAAEAKKKAVKESSIRSVHETVLPIKKRKTRETVSIEVKEVVKPLLVSTLGEKSGKGLKTCKSPGRKSKESSPKGRSSSASSPPKKEHHHHHHHSESTKAPMPLLPSPPPPEPESSEDPISPPEPQDLSSSICKEEKMPRGGSLESDGCPKEPAKTQPMVATTTTVAEKYKHRGEGERKDIVSSSMPRPNREEPVDSRTPVTERVS

SEQ ID NO: 30 - protein, 484 aa, Mus musculus
MVAGMLGLREEKSEDQDLQGLRDKPLKFKKAKKDKKEDKEGKHEPLQPSAHHSAEPAEAGKAETSESSGSAPAVPEASASPKQRRSIIRDRGPMYDDPTLPEGWTRKLKQRKSGRSAGKYDVYLINPQGKAFRSKVELIAYFEKVGDTSLDPNDFDFTVTGRGSPSRREQKPPKKPKSPKAPGTGRGRGRPKGSGTGRPKAAASEGVQVKRVLEKSPGKLVVKMPFQASPGGKGEGGGATTSAQVMVIKRPGRKRKAEADPQAIPKKRGRKPGSVVAAAAAEAKKKAVKESSIRSVHETVLPIKKRKTRETVSIEVKEVVKPLLVSTLGEKSGKGLKTCKSPGRKSKESSPKGRSSSASSPPKKEHHHHHHHSESTKAPMPLLPSPPPPEPESSEDPISPPEPQDLSSSICKEEKMPRGGSLESDGCPKEPAKTQPMVATTTTVAEKYKHRGEGERKDIVSSSMPRPNREEPVDSRTPVTERVS

SEQ ID NO: 31 - DNA, 1452 bp, Mus musculus
  source 1..1452: mol_type = genomic DNA
atggtagctgggatgttagggctcagggaggaaaagtcagaagaccaggatctccagggcctcagagacaagccactgaagtttaagaaggcgaagaaagacaagaaggaggacaaagaaggcaagcatgagccactacaaccttcagcccaccattctgcagagccagcagaggcaggcaaagcagaaacatcagaaagctcaggctctgccccagcagtgccagaagcctcggcttcccccaaacagcggcgctccattatccgtgaccggggacctatgtatgatgaccccaccttgcctgaaggttggacacgaaagcttaaacaaaggaagtctggccgatctgctggaaagtatgatgtatatttgatcaatccccagggaaaagcttttcgctctaaagtagaattgattgcatactttgaaaaggtgggagacacctccttggaccctaatgattttgacttcacggtaactgggagagggagcccctccaggagagagcagaaaccacctaagaagcccaaatctcccaaagctccaggaactggcaggggtcggggacgccccaaagggagcggcactgggagaccaaaggcagcagcatcagaaggtgttcaggtgaaaagggtcctggagaagagccctgggaaacttgttgtcaagatgcctttccaagcatcgcctgggggtaagggtgagggaggtggggctaccacatctgcccaggtcatggtgatcaaacgccctggcagaaagcgaaaagctgaagctgacccccaggccattcctaagaaacggggtagaaagcctgggagtgtggtggcagctgctgcagctgaggccaaaaagaaagccgtgaaggagtcttccatacggtctgtgcatgagactgtgctccccatcaagaagcgcaagacccgggagacggtcagcatcgaggtcaaggaagtggtgaagcccctgctggtgtccacccttggtgagaaaagcgggaagggactgaagacctgcaagagccctgggcgtaaaagcaaggagagcagccccaaggggcgcagcagcagtgcctcctccccacctaagaaggagcaccatcatcaccaccatcactcagagtccacaaaggcccccatgccactgctcccatccccacccccacctgagcctgagagctctgaggaccccatcagcccccctgagcctcaggacttgagcagcagcatctgcaaagaagagaagatgccccgaggaggctcactggaaagcgatggctgccccaaggagccagctaagactcagcctatggtcgccaccactaccacagttgcagaaaagtacaaacaccgaggggagggagagcgcaaagacattgtttcatcttccatgccaaggccaaacagagaggagcctgtggacagccggacgcccgtgaccgagagagttagc

SEQ ID NO: 32 - protein, 7 aa, synthetic construct
  REGION 1..7: note = Nuclear localisation sequence (NLS) polypeptide
PKKKRKV

SEQ ID NO: 33 - DNA, 21 bp, synthetic construct
  misc_feature 1..21: note = polynucleotide sequence encoding NLS
  source 1..21: mol_type = other DNA
cccaagaaaaagcggaaggtg

SEQ ID NO: 34 - protein, 7 aa, synthetic construct
  REGION 1..7: note = Linker polypeptide sequence
GSSGSSG

SEQ ID NO: 35 - DNA, 21 bp, synthetic construct
  misc_feature 1..21: note = polynucleotide sequence encoding linker
  source 1..21: mol_type = other DNA
ggatccagtggcagctctggg

SEQ ID NO: 36 - protein, 8 aa, synthetic construct
  REGION 1..8: note = Linker polypeptide sequence
CKDPPVAT

SEQ ID NO: 37 - DNA, 24 bp, synthetic construct
  misc_feature 1..24: note = Polynucleotide sequence encoding linker
  source 1..24: mol_type = other DNA
tgtaaggatccaccggtcgccacc

SEQ ID NO: 38 - protein, 7 aa, synthetic construct
  REGION 1..7: note = linker polypeptide sequence
GSSGSSG

SEQ ID NO: 39 - DNA, 21 bp, synthetic construct
  misc_feature 1..21: note = polynucleotide sequence encoding linker
  source 1..21: mol_type = other DNA
gggagctccggcagttctgga

SEQ ID NO: 40 - protein, 748 aa, synthetic construct
  REGION 1..748: note = Mouse WT e1 with EGFP tag polypeptide
MAAAAATAAAAAAPSGGGGGGEEERLEEKSEDQDLQGLRDKPLKFKKAKKDKKEDKEGKHEPLQPSAHHSAEPAEAGKAETSESSGSAPAVPEASASPKQRRSIIRDRGPMYDDPTLPEGWTRKLKQRKSGRSAGKYDVYLINPQGKAFRSKVELIAYFEKVGDTSLDPNDFDFTVTGRGSPSRREQKPPKKPKSPKAPGTGRGRGRPKGSGTGRPKAAASEGVQVKRVLEKSPGKLVVKMPFQASPGGKGEGGGATTSAQVMVIKRPGRKRKAEADPQAIPKKRGRKPGSVVAAAAAEAKKKAVKESSIRSVHETVLPIKKRKTRETVSIEVKEVVKPLLVSTLGEKSGKGLKTCKSPGRKSKESSPKGRSSSASSPPKKEHHHHHHHSESTKAPMPLLPSPPPPEPESSEDPISPPEPQDLSSSICKEEKMPRGGSLESDGCPKEPAKTQPMVATTTTVAEKYKHRGEGERKDIVSSSMPRPNREEPVDSRTPVTERVSCKDPPVATMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 41 - protein, 689 aa, synthetic construct
  REGION 1..689: note = Mouse dN e1 with EGFP tag polypeptide
MAAAAATAAAAAAPSGGGGGGEEERLEEKPAVPEASASPKQRRSIIRDRGPMYDDPTLPEGWTRKLKQRKSGRSAGKYDVYLINPQGKAFRSKVELIAYFEKVGDTSLDPNDFDFTVTGRGSPSRREQKPPKKPKSPKAPGTGRGRGRPKGSGTGRPKAAASEGVQVKRVLEKSPGKLVVKMPFQASPGGKGEGGGATTSAQVMVIKRPGRKRKAEADPQAIPKKRGRKPGSVVAAAAAEAKKKAVKESSIRSVHETVLPIKKRKTRETVSIEVKEVVKPLLVSTLGEKSGKGLKTCKSPGRKSKESSPKGRSSSASSPPKKEHHHHHHHSESTKAPMPLLPSPPPPEPESSEDPISPPEPQDLSSSICKEEKMPRGGSLESDGCPKEPAKTQPMVATTTTVAEKYKHRGEGERKDIVSSSMPRPNREEPVDSRTPVTERVSCKDPPVATMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 42 - protein, 516 aa, synthetic construct
  REGION 1..516: note = Mouse dNC e1 with EGFP tag polypeptide
MAAAAATAAAAAAPSGGGGGGEEERLEEKPAVPEASASPKQRRSIIRDRGPMYDDPTLPEGWTRKLKQRKSGRSAGKYDVYLINPQGKAFRSKVELIAYFEKVGDTSLDPNDFDFTVTGRGSPSRREQKPPKKPKSPKAPGTGRGRGRPKGSGTGRPKAAASEGVQVKRVLEKSPGKLVVKMPFQASPGGKGEGGGATTSAQVMVIKRPGRKRKAEADPQAIPKKRGRKPGSVVAAAAAEAKKKAVKESSIRSVHETVLPIKKRKTRETVGSSGSSGMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 43 - protein, 432 aa, synthetic construct
  REGION 1..432: note = Mouse dNIC e1 with EGFP tag polypeptide
MAAAAATAAAAAAPSGGGGGGEEERLEEKPAVPEASASPKQRRSIIRDRGPMYDDPTLPEGWTRKLKQRKSGRSAGKYDVYLINPQGKAFRSKVELIAYFEKVGDTSLDPNDFDFTVTGRGSPSRREQKPPGSSGSSGPKKKRKVPGSVVAAAAAEAKKKAVKESSIRSVHETVLPIKKRKTRETVGSSGSSGMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 44 - protein, 731 aa, synthetic construct
  REGION 1..731: note = Mouse WT e2 with EGFP tag polypeptide
MVAGMLGLREEKSEDQDLQGLRDKPLKFKKAKKDKKEDKEGKHEPLQPSAHHSAEPAEAGKAETSESSGSAPAVPEASASPKQRRSIIRDRGPMYDDPTLPEGWTRKLKQRKSGRSAGKYDVYLINPQGKAFRSKVELIAYFEKVGDTSLDPNDFDFTVTGRGSPSRREQKPPKKPKSPKAPGTGRGRGRPKGSGTGRPKAAASEGVQVKRVLEKSPGKLVVKMPFQASPGGKGEGGGATTSAQVMVIKRPGRKRKAEADPQAIPKKRGRKPGSVVAAAAAEAKKKAVKESSIRSVHETVLPIKKRKTRETVSIEVKEVVKPLLVSTLGEKSGKGLKTCKSPGRKSKESSPKGRSSSASSPPKKEHHHHHHHSESTKAPMPLLPSPPPPEPESSEDPISPPEPQDLSSSICKEEKMPRGGSLESDGCPKEPAKTQPMVATTTTVAEKYKHRGEGERKDIVSSSMPRPNREEPVDSRTPVTERVSCKDPPVATMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 45 - protein, 672 aa, synthetic construct
  REGION 1..672: note = Mouse dN e2 with EGFP tag polypeptide
MVAGMLGLREEKPAVPEASASPKQRRSIIRDRGPMYDDPTLPEGWTRKLKQRKSGRSAGKYDVYLINPQGKAFRSKVELIAYFEKVGDTSLDPNDFDFTVTGRGSPSRREQKPPKKPKSPKAPGTGRGRGRPKGSGTGRPKAAASEGVQVKRVLEKSPGKLVVKMPFQASPGGKGEGGGATTSAQVMVIKRPGRKRKAEADPQAIPKKRGRKPGSVVAAAAAEAKKKAVKESSIRSVHETVLPIKKRKTRETVSIEVKEVVKPLLVSTLGEKSGKGLKTCKSPGRKSKESSPKGRSSSASSPPKKEHHHHHHHSESTKAPMPLLPSPPPPEPESSEDPISPPEPQDLSSSICKEEKMPRGGSLESDGCPKEPAKTQPMVATTTTVAEKYKHRGEGERKDIVSSSMPRPNREEPVDSRTPVTERVSCKDPPVATMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 46 - protein, 499 aa, synthetic construct
  REGION 1..499: note = Mouse dNC e2 with EGFP tag polypeptide
MVAGMLGLREEKPAVPEASASPKQRRSIIRDRGPMYDDPTLPEGWTRKLKQRKSGRSAGKYDVYLINPQGKAFRSKVELIAYFEKVGDTSLDPNDFDFTVTGRGSPSRREQKPPKKPKSPKAPGTGRGRGRPKGSGTGRPKAAASEGVQVKRVLEKSPGKLVVKMPFQASPGGKGEGGGATTSAQVMVIKRPGRKRKAEADPQAIPKKRGRKPGSVVAAAAAEAKKKAVKESSIRSVHETVLPIKKRKTRETVGSSGSSGMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 47 - protein, 415 aa, synthetic construct
  REGION 1..415: note = Mouse dNIC e2 with EGFP tag polypeptide
MVAGMLGLREEKPAVPEASASPKQRRSIIRDRGPMYDDPTLPEGWTRKLKQRKSGRSAGKYDVYLINPQGKAFRSKVELIAYFEKVGDTSLDPNDFDFTVTGRGSPSRREQKPPGSSGSSGPKKKRKVPGSVVAAAAAEAKKKAVKESSIRSVHETVLPIKKRKTRETVGSSGSSGMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 48 - DNA, 2274 bp, synthetic construct
  misc_feature 1..2274: note = Mouse WT e1 with EGFP tag cDNA
  source 1..2274: mol_type = other DNA
atggccgccgctgccgccaccgccgccgccgccgccgcgccgagcggaggaggaggaggaggcgaggaggagagactggaggaaaagtcagaagaccaggatctccagggcctcagagacaagccactgaagtttaagaaggcgaagaaagacaagaaggaggacaaagaaggcaagcatgagccactacaaccttcagcccaccattctgcagagccagcagaggcaggcaaagcagaaacatcagaaagctcaggctctgccccagcagtgccagaagcctcggcttcccccaaacagcggcgctccattatccgtgaccggggacctatgtatgatgaccccaccttgcctgaaggttggacacgaaagcttaaacaaaggaagtctggccgatctgctggaaagtatgatgtatatttgatcaatccccagggaaaagcttttcgctctaaagtagaattgattgcatactttgaaaaggtgggagacacctccttggaccctaatgattttgacttcacggtaactgggagagggagcccctccaggagagagcagaaaccacctaagaagcccaaatctcccaaagctccaggaactggcaggggtcggggacgccccaaagggagcggcactgggagaccaaaggcagcagcatcagaaggtgttcaggtgaaaagggtcctggagaagagccctgggaaacttgttgtcaagatgcctttccaagcatcgcctgggggtaagggtgagggaggtggggctaccacatctgcccaggtcatggtgatcaaacgccctggcagaaagcgaaaagctgaagctgacccccaggccattcctaagaaacggggtagaaagcctgggagtgtggtggcagctgctgcagctgaggccaaaaagaaagccgtgaaggagtcttccatacggtctgtgcatgagactgtgctccccatcaagaagcgcaagacccgggagacggtcagcatcgaggtcaaggaagtggtgaagcccctgctggtgtccacccttggtgagaaaagcgggaagggactgaagacctgcaagagccctgggcgtaaaagcaaggagagcagccccaaggggcgcagcagcagtgcctcctccccacctaagaaggagcaccatcatcaccaccatcactcagagtccacaaaggcccccatgccactgctcccatccccacccccacctgagcctgagagctctgaggaccccatcagcccccctgagcctcaggacttgagcagcagcatctgcaaagaagagaagatgccccgaggaggctcactggaaagcgatggctgccccaaggagccagctaagactcagcctatggtcgccaccactaccacagttgcagaaaagtacaaacaccgaggggagggagagcgcaaagacattgtttcatcttccatgccaaggccaaacagagaggagcctgtggacagccggacgcccgtgaccgagagagttagctgtaaggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaactcggcatggacgagctgtacaagtaa

SEQ ID NO: 49 - DNA, 2070 bp, synthetic construct
  misc_feature 1..2070: note = Mouse dN e1 with EGFP tag cDNA
  source 1..2070: mol_type = other DNA
atggccgccgctgccgccaccgccgccgccgccgccgcgccgagcggaggaggaggaggaggcgaggaggagagactggaggaaaagccagcagtgccagaagcctcggcttcccccaaacagcggcgctccattatccgtgaccggggacctatgtatgatgaccccaccttgcctgaaggttggacacgaaagcttaaacaaaggaagtctggccgatctgctggaaagtatgatgtatatttgatcaatccccagggaaaagcttttcgctctaaagtagaattgattgcatactttgaaaaggtgggagacacctccttggaccctaatgattttgacttcacggtaactgggagagggagcccctccaggagagagcagaaaccacctaagaagcccaaatctcccaaagctccaggaactggcaggggtcggggacgccccaaagggagcggcactgggagaccaaaggcagcagcatcagaaggtgttcaggtgaaaagggtcctggagaagagccctgggaaacttgttgtcaagatgcctttccaagcatcgcctgggggtaagggtgagggaggtggggctaccacatctgcccaggtcatggtgatcaaacgccctggcagaaagcgaaaagctgaagctgacccccaggccattcctaagaaacggggtagaaagcctgggagtgtggtggcagctgctgcagctgaggccaaaaagaaagccgtgaaggagtcttccatacggtctgtgcatgagactgtgctccccatcaagaagcgcaagacccgggagacggtcagcatcgaggtcaaggaagtggtgaagcccctgctggtgtccacccttggtgagaaaagcgggaagggactgaagacctgcaagagccctgggcgtaaaagcaaggagagcagccccaaggggcgcagcagcagtgcctcctccccacctaagaaggagcaccatcatcaccaccatcactcagagtccacaaaggcccccatgccactgctcccatccccacccccacctgagcctgagagctctgaggaccccatcagcccccctgagcctcaggacttgagcagcagcatctgcaaagaagagaagatgccccgaggaggctcactggaaagcgatggctgccccaaggagccagctaagactcagcctatggtcgccaccactaccacagttgcagaaaagtacaaacaccgaggggagggagagcgcaaagacattgtttcatcttccatgccaaggccaaacagagaggagcctgtggacagccggacgcccgtgaccgagagagttagctgtaaggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 50 - DNA, 1551 bp, synthetic construct
  misc_feature 1..1551: note = Mouse dNC e1 with EGFP tag cDNA
  source 1..1551: mol_type = other DNA
atggccgccgctgccgccaccgccgccgccgccgccgcgccgagcggaggaggaggaggaggcgaggaggagagactggaggaaaagccagcagtgccagaagcctcggcttcccccaaacagcggcgctccattatccgtgaccggggacctatgtatgatgaccccaccttgcctgaaggttggacacgaaagcttaaacaaaggaagtctggccgatctgctggaaagtatgatgtatatttgatcaatccccagggaaaagcttttcgctctaaagtagaattgattgcatactttgaaaaggtgggagacacctccttggaccctaatgattttgacttcacggtaactgggagagggagcccctccaggagagagcagaaaccacctaagaagcccaaatctcccaaagctccaggaactggcaggggtcggggacgccccaaagggagcggcactgggagaccaaaggcagcagcatcagaaggtgttcaggtgaaaagggtcctggagaagagccctgggaaacttgttgtcaagatgcctttccaagcatcgcctgggggtaagggtgagggaggtggggctaccacatctgcccaggtcatggtgatcaaacgccctggcagaaagcgaaaagctgaagctgacccccaggccattcctaagaaacggggtagaaagcctgggagtgtggtggcagctgctgcagctgaggccaaaaagaaagccgtgaaggagtcttccatacggtctgtgcatgagactgtgctccccatcaagaagcgcaagacccgggagacggtcgggagctccggcagttctggaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 51 - DNA, 1299 bp, synthetic construct
  misc_feature 1..1299: note = Mouse dNIC e1 with EGFP tag cDNA
  source 1..1299: mol_type = other DNA
atggccgccgctgccgccaccgccgccgccgccgccgcgccgagcggaggaggaggaggaggcgaggaggagagactggaggaaaagccagcagtgccagaagcctcggcttcccccaaacagcggcgctccattatccgtgaccggggacctatgtatgatgaccccaccttgcctgaaggttggacacgaaagcttaaacaaaggaagtctggccgatctgctggaaagtatgatgtatatttgatcaatccccagggaaaagcttttcgctctaaagtagaattgattgcatactttgaaaaggtgggagacacctccttggaccctaatgattttgacttcacggtaactgggagagggagcccctccaggagagagcagaaaccacctggatccagtggcagctctgggcccaagaaaaagcggaaggtgcctgggagtgtggtggcagctgctgcagctgaggccaaaaagaaagccgtgaaggagtcttccatacggtctgtgcatgagactgtgctccccatcaagaagcgcaagacccgggagacggtcgggagctccggcagttctggaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 52 - DNA, 2196 bp, synthetic construct
  misc_feature 1..2196: note = Mouse WT e2 with EGFP tag cDNA
  source 1..2196: mol_type = other DNA
atggtagctgggatgttagggctcagggaggaaaagtcagaagaccaggatctccagggcctcagagacaagccactgaagtttaagaaggcgaagaaagacaagaaggaggacaaagaaggcaagcatgagccactacaaccttcagcccaccattctgcagagccagcagaggcaggcaaagcagaaacatcagaaagctcaggctctgccccagcagtgccagaagcctcggcttcccccaaacagcggcgctccattatccgtgaccggggacctatgtatgatgaccccaccttgcctgaaggttggacacgaaagcttaaacaaaggaagtctggccgatctgctggaaagtatgatgtatatttgatcaatccccagggaaaagcttttcgctctaaagtagaattgattgcatactttgaaaaggtgggagacacctccttggaccctaatgattttgacttcacggtaactgggagagggagcccctccaggagagagcagaaaccacctaagaagcccaaatctcccaaagctccaggaactggcaggggtcggggacgccccaaagggagcggcactgggagaccaaaggcagcagcatcagaaggtgttcaggtgaaaagggtcctggagaagagccctgggaaacttgttgtcaagatgcctttccaagcatcgcctgggggtaagggtgagggaggtggggctaccacatctgcccaggtcatggtgatcaaacgccctggcagaaagcgaaaagctgaagctgacccccaggccattcctaagaaacggggtagaaagcctgggagtgtggtggcagctgctgcagctgaggccaaaaagaaagccgtgaaggagtcttccatacggtctgtgcatgagactgtgctccccatcaagaagcgcaagacccgggagacggtcagcatcgaggtcaaggaagtggtgaagcccctgctggtgtccacccttggtgagaaaagcgggaagggactgaagacctgcaagagccctgggcgtaaaagcaaggagagcagccccaaggggcgcagcagcagtgcctcctccccacctaagaaggagcaccatcatcaccaccatcactcagagtccacaaaggcccccatgccactgctcccatccccacccccacctgagcctgagagctctgaggaccccatcagcccccctgagcctcaggacttgagcagcagcatctgcaaagaagagaagatgccccgaggaggctcactggaaagcgatggctgccccaaggagccagctaagactcagcctatggtcgccaccactaccacagttgcagaaaagtacaaacaccgaggggagggagagcgcaaagacattgtttcatcttccatgccaaggccaaacagagaggagcctgtggacagccggacgcccgtgaccgagagagttagctgtaaggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 53 - DNA, 2019 bp, synthetic construct
  misc_feature 1..2019: note = Mouse dN e2 with EGFP tag cDNA
  source 1..2019: mol_type = other DNA
atggtagctgggatgttagggctcagggaggaaaagccagcagtgccagaagcctcggcttcccccaaacagcggcgctccattatccgtgaccggggacctatgtatgatgaccccaccttgcctgaaggttggacacgaaagcttaaacaaaggaagtctggccgatctgctggaaagtatgatgtatatttgatcaatccccagggaaaagcttttcgctctaaagtagaattgattgcatactttgaaaaggtgggagacacctccttggaccctaatgattttgacttcacggtaactgggagagggagcccctccaggagagagcagaaaccacctaagaagcccaaatctcccaaagctccaggaactggcaggggtcggggacgccccaaagggagcggcactgggagaccaaaggcagcagcatcagaaggtgttcaggtgaaaagggtcctggagaagagccctgggaaacttgttgtcaagatgcctttccaagcatcgcctgggggtaagggtgagggaggtggggctaccacatctgcccaggtcatggtgatcaaacgccctggcagaaagcgaaaagctgaagctgacccccaggccattcctaagaaacggggtagaaagcctgggagtgtggtggcagctgctgcagctgaggccaaaaagaaagccgtgaaggagtcttccatacggtctgtgcatgagactgtgctccccatcaagaagcgcaagacccgggagacggtcagcatcgaggtcaaggaagtggtgaagcccctgctggtgtccacccttggtgagaaaagcgggaagggactgaagacctgcaagagccctgggcgtaaaagcaaggagagcagccccaaggggcgcagcagcagtgcctcctccccacctaagaaggagcaccatcatcaccaccatcactcagagtccacaaaggcccccatgccactgctcccatccccacccccacctgagcctgagagctctgaggaccccatcagcccccctgagcctcaggacttgagcagcagcatctgcaaagaagagaagatgccccgaggaggctcactggaaagcgatggctgccccaaggagccagctaagactcagcctatggtcgccaccactaccacagttgcagaaaagtacaaacaccgaggggagggagagcgcaaagacattgtttcatcttccatgccaaggccaaacagagaggagcctgtggacagccggacgcccgtgaccgagagagttagctgtaaggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 54 - DNA, 1500 bp, synthetic construct
  misc_feature 1..1500: note = Mouse dNC e2 with EGFP tag cDNA
  source 1..1500: mol_type = other DNA
atggtagctgggatgttagggctcagggaggaaaagccagcagtgccagaagcctcggcttcccccaaacagcggcgctccattatccgtgaccggggacctatgtatgatgaccccaccttgcctgaaggttggacacgaaagcttaaacaaaggaagtctggccgatctgctggaaagtatgatgtatatttgatcaatccccagggaaaagcttttcgctctaaagtagaattgattgcatactttgaaaaggtgggagacacctccttggaccctaatgattttgacttcacggtaactgggagagggagcccctccaggagagagcagaaaccacctaagaagcccaaatctcccaaagctccaggaactggcaggggtcggggacgccccaaagggagcggcactgggagaccaaaggcagcagcatcagaaggtgttcaggtgaaaagggtcctggagaagagccctgggaaacttgttgtcaagatgcctttccaagcatcgcctgggggtaagggtgagggaggtggggctaccacatctgcccaggtcatggtgatcaaacgccctggcagaaagcgaaaagctgaagctgacccccaggccattcctaagaaacggggtagaaagcctgggagtgtggtggcagctgctgcagctgaggccaaaaagaaagccgtgaaggagtcttccatacggtctgtgcatgagactgtgctccccatcaagaagcgcaagacccgggagacggtcgggagctccggcagttctggaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 55 - DNA, 1248 bp, synthetic construct
  misc_feature 1..1248: note = Mouse dNIC e2 with EGFP tag cDNA
  source 1..1248: mol_type = other DNA
atggtagctgggatgttagggctcagggaggaaaagccagcagtgccagaagcctcggcttcccccaaacagcggcgctccattatccgtgaccggggacctatgtatgatgaccccaccttgcctgaaggttggacacgaaagcttaaacaaaggaagtctggccgatctgctggaaagtatgatgtatatttgatcaatccccagggaaaagcttttcgctctaaagtagaattgattgcatactttgaaaaggtgggagacacctccttggaccctaatgattttgacttcacggtaactgggagagggagcccctccaggagagagcagaaaccacctggatccagtggcagctctgggcccaagaaaaagcggaaggtgcctgggagtgtggtggcagctgctgcagctgaggccaaaaagaaagccgtgaaggagtcttccatacggtctgtgcatgagactgtgctccccatcaagaagcgcaagacccgggagacggtcgggagctccggcagttctggaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 56 - DNA, 34 bp, synthetic construct
  misc_feature 1..34: note = Primer: R111G Forward
  source 1..34: mol_type = other DNA
tggacacgaaagcttaaacaagggaagtctggcc

SEQ ID NO: 57 - DNA, 34 bp, synthetic construct
  misc_feature 1..34: note = Primer: R111G Reverse
  source 1..34: mol_type = other DNA
ggccagacttcccttgtttaagctttcgtgtcca

SEQ ID NO: 58 - DNA, 29 bp, synthetic construct
  misc_feature 1..29: note = Primer: R306C Forward
  source 1..29: mol_type = other DNA
ctcccgggtcttgcacttcttgatgggga

SEQ ID NO: 59 - DNA, 29 bp, synthetic construct
  misc_feature 1..29: note = Primer: R306C Reverse
  source 1..29: mol_type = other DNA
tccccatcaagaagtgcaagacccgggag

SEQ ID NO: 60 - protein, 284 aa, synthetic construct
  REGION 1..284: note = Human dNC e1 with Myc tag polypeptide
MAAAAAAAPSGGGGGGEEERLEEKPAVPEASASPKQRRSIIRDRGPMYDDPTLPEGWTRKLKQRKSGRSAGKYDVYLINPQGKAFRSKVELIAYFEKVGDTSLDPNDFDFTVTGRGSPSRREQKPPKKPKSPKAPGTGRGRGRPKGSGTTRPKAATSEGVQVKRVLEKSPGKLLVKMPFQTSPGGKAEGGGATTSTQVMVIKRPGRKRKAEADPQAIPKKRGRKPGSVVAAAAAEAKKKAVKESSIRSVQETVLPIKKRKTRETVGSSGSSGEQKLISEEDLVD

SEQ ID NO: 61 - protein, 200 aa, synthetic construct
  REGION 1..200: note = Human dNIC e1 with Myc tag polypeptide
MAAAAAAAPSGGGGGGEEERLEEKPAVPEASASPKQRRSIIRDRGPMYDDPTLPEGWTRKLKQRKSGRSAGKYDVYLINPQGKAFRSKVELIAYFEKVGDTSLDPNDFDFTVTGRGSPSRREQKPPGSSGSSGPKKKRKVPGSVVAAAAAEAKKKAVKESSIRSVQETVLPIKKRKTRETVGSSGSSGEQKLISEEDLVD

SEQ ID NO: 62 - DNA, 855 bp, synthetic construct
  misc_feature 1..855: note = Human dNC e1 with Myc tag cDNA
  source 1..855: mol_type = other DNA
atggccgccgccgccgccgccgcgccgagcggaggaggaggaggaggcgaggaggagagactggaagaaaagccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaatccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcgggagctccggcagttctggagaacaaaaactcatctcagaagaggatctggtcgactag

SEQ ID NO: 63 - DNA, 603 bp, synthetic construct
  misc_feature 1..603: note = Human dNIC e1 with Myc tag cDNA
  source 1..603: mol_type = other DNA
atggccgccgccgccgccgccgcgccgagcggaggaggaggaggaggcgaggaggagagactggaagaaaagccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaatccccagggaaaagcctttcgctctaaagtggagttgattgcgtacttcgaaaaggtaggcgacacatccctggaccctaatgattttgacttcacggtaactgggagagggagcccctcccggcgagagcagaaaccacctggatccagtggcagctctgggcccaagaaaaagcggaaggtgccggggagtgtggtggcagccgctgccgccgaggccaaaaagaaagccgtgaaggagtcttctatccgatctgtgcaggagaccgtactccccatcaagaagcgcaagacccgggagacggtcgggagctccggcagttctggagaacaaaaactcatctcagaagaggatctggtcgactag

SEQ ID NO: 64 - DNA, 20 bp, synthetic construct
  misc_feature 1..20: note = Guide sequence RNA
  source 1..20: mol_type = other DNA
ggttgtgacccgccatggat

SEQ ID NO: 65 - DNA, 1504 bp, synthetic construct
  misc_feature 1..1504: note = dNIC expression cassette flanked by AAV2 ITRs
  source 1..1504: mol_type = other DNA
gcgcgctcgctcgctcactgaggccgcccgggcaaagcccgggcgtcgggcgacctttggtcgcccggcctcagtgagcgagcgagcgcgcagagagggagtggggttcggtacccataggcgccaagagcctagacttccttaagcgccagagtccacaagggcccagttaatcctcaacattcaaatgctgcccacaaaaccagcccctctgtgccctagccgcctcttttttccaagtgacagtagaactccaccaatccgcagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcgcgctccctcctctcggagagagggctgtggtaaaacccgtccggaaaccatggccgccgccgccgccgccgcgccgagcggaggaggaggaggaggcgaggaggagagactggaagaaaagccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaatccccagggaaaagcctttcgctctaaagtggagttgattgcgtacttcgaaaaggtaggcgacacatccctggaccctaatgattttgacttcacggtaactgggagagggagcccctcccggcgagagcagaaaccacctggatccagtggcagctctgggcccaagaaaaagcggaaggtgccggggagtgtggtggcagccgctgccgccgaggccaaaaagaaagccgtgaaggagtcttctatccgatctgtgcaggagaccgtactccccatcaagaagcgcaagacccgggagacggtcgggagctccggcagttctggagaacaaaaactcatctcagaagaggatctggtcgactagagctcgctgatcagcctcacaagaataaaggcagctgttgtctcttcagaagtagctttgcacttttctaaactaggaatatcaccaggactgttactcaatgtgtgggtaccgaaagcactgatatatttaaaaacaaaaggtgtaacctatttattatataaagagtttgccttataaatttacataaaaatgtccgtttgtgtcttttgttgtaaaaatcacgcgtaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 66 - protein, 126 aa, Pan paniscus
SAPAVPEASASPKQRRSIIRDRGPMYDDPTLPEGWTRKLKQRKSGRSAGKYDVYLINPQGKAFRSKVELIAYFEKVGDTSLDPNDFDFTVTGRGSPSRREQKPPKKPKSPKAPGTGRGRGRPKGSG

SEQ ID NO: 67 - protein, 126 aa, Xenopus laevis
ENLAVPESSASPKQRRSVIRDRGPMYEDPTLPEGWTRKLKQRKSGRSAGKFDVYLINPNGKAFRSKVELIAYFQKVGDTSLDPNDFDFTVTGRGSPSRREQKQPKKPKAPKSSVSGRGRGRPKGSI

SEQ ID NO: 68 - protein, 124 aa, Danio rerio
AALSAPESSASAKQRRSVIRDRGPMYEDPSLPQGWTRKLKQRKSGRSAGKFDVYLINPEGKAFRSKVELMAYFQKVGDTITDPNDFDFTVTGRGSPSRREKRPPKKPKMVKPSGRGRGRPKGSG

SEQ ID NO: 69 - protein, 72 aa, Pan paniscus
RKRKAEADPQAIPKKRGRKPGSVVAAAAAEAKKKAVKESSIRSVQETVLPIKKRKTRETVSIEVKEVVKPLL

SEQ ID NO: 70 - protein, 74 aa, Xenopus laevis
RKRKSETDPSAAPKKRGRKPSNVSLAAAAAEAAKKKAIKESSIKPLLETVLPIKKRKTRETISVDVKDTIKPEP

SEQ ID NO: 71 - protein, 92 aa, Danio rerio
RKRKSEQDPPSTPKKRGRKPATVSQSTVGTGSAAAYAAAAILTAEAKKKALKESSAKPVQERALPIKKRKTRETLEELEASTTSATETFEKR